SEQUENCE LISTING (35 sequences)

SEQ ID NO: 1 - DNA, 22 bp, Artificial Sequence
  RACE Primer 5′ 62361 Outer
tccacctcatcttccggacgaa

SEQ ID NO: 2 - DNA, 21 bp, Artificial Sequence
  RACE Primer 5′ 62361 Inner
acttccgttccagagcgtgtt

SEQ ID NO: 3 - DNA, 23 bp, Artificial Sequence
  RACE Primer 3′ 62361 Outer
agaagacctgtacgaccatgcat

SEQ ID NO: 4 - DNA, 24 bp, Artificial Sequence
  RACE Primer 3′ 62361 Inner
acgagcgattagtgaagctcgtca

SEQ ID NO: 5 - DNA, 22 bp, Artificial Sequence
  PCR Primer Forward Outer
cttcacttgtgtcaagttcaat

SEQ ID NO: 6 - DNA, 23 bp, Artificial Sequence
  PCR Primer Reverse Outer
ccratagatagcatgcagaatct

SEQ ID NO: 7 - DNA, 29 bp, Artificial Sequence
  PCR Primer Forward Inner
aattcatatgggtgctagcatagatgatt

SEQ ID NO: 8 - DNA, 36 bp, Artificial Sequence
  PCR Primer Reverse Inner
aattgcggccgctcaataaagacgtcggcaaatagt

SEQ ID NO: 9 - DNA, 22 bp, Artificial Sequence
  RT PCR Primer Forward 62361
attggtgtgtacaccggctatt

SEQ ID NO: 10 - DNA, 23 bp, Artificial Sequence
  RT PCR Primer Reverse 62361
ttccatcttccggtaaagccaaa

SEQ ID NO: 11 - DNA, 16 bp, Artificial Sequence
  RT PCR Primer Probe 62361
ctctgctcacaactgc

SEQ ID NO: 12 - DNA, 17 bp, Artificial Sequence
  PCR Primer T7 Sequencing
taatacgactcactata

SEQ ID NO: 13 - DNA, 19 bp, Artificial Sequence
  PCR Primer T7 Terminator
gctagttattgctcagcgg

SEQ ID NO: 14 - DNA, 720 bp, Narcissus spp. norbelladine
atgggtgctagcatagatgattattccctagtccacaagaacatcttgcatagtgaagatcttctgaagtacatattggagactagtgcataccctagagagcatgaacagctcaaggggctgagggaggtgactgagaaacatgaatggagttcggcgcttgtcccagccgatgaaggattgtttctttctatgctgttaaagctcatgaatgctaagagaaccattgagattggtgtgtacaccggctattctctgctcacaactgctttggctttaccggaagatggaaagataacggcaattgatgtcaacaagtcctactatgagataggactaccatttattcagaaagcaggagttgagcataagatcaatttcattgaatcagaagcacttcctgttcttgatcaaatgcttgaagagatgaaggaagaagacctgtacgactatgcatttgtcgatgcagacaaatctaactacgctaattaccacgagcgattagtgaagctcgtcaggattggaggggcgatcctctacgacaacacgctctggtacggaagtgtagcatatccagaatatccaggccttcatccggaagaggaggtggctaggctctctttcagaaacctgaacactttcttggcagctgatccccgtgtcgagatatcccaggtctctatcggcgacggagtgactatttgccgacgtctttattga

SEQ ID NO: 15 - protein, 239 aa, Narcissus spp. norbelladine
MGASIDDYSLVHKNILHSEDLLKYILETSAYPREHEQLKGLREVTEKHEWSSALVPADEGLFLSMLLKLMNAKRTIEIGVYTGYSLLTTALALPEDGKITAIDVNKSYYEIGLPFIQKAGVEHKINFIESEALPVLDQMLEEMKEEDLYDYAFVDADKSNYANYHERLVKLVRIGGAILYDNTLWYGSVAYPEYPGLHPEEEVARLSFRNLNTFLAADPRVEISQVSIGDGVTICRRLY

SEQ ID NO: 16 - DNA, 720 bp, Narcissus spp. norbelladine
atgggtgctagcatagatgattattccctagtccacaagaacatcttgcatagtgaagatcttctgaagtacatattggagactagtgcataccctagagagcatgaacagctcaaggggctgagggaggtcactgagaaacatgaatggagttcagcgcttgtcccagccgatgaaggattgtttctttctatgttgttaaagctcatgaatgctaagagaaccattgagattggtgtgtacaccggctattctctgctcacaactgctttggctttaccggaagatggaaagataacggcaatcgatgtcaacaagtccttctttgagataggactaccatttattcagaaagcaggagttgagcataagataaatttcattgaatcagaggcacttcctgttcttgatcaaatgcttcaagagacgaaggaagaagacctgtacgactatgcatttgtcgatgcagacaaatctaactacgctaattaccacgagcgattagtgaagctcgtcaggattggaggggcgatcctctacgacaacacgctctggtacggaagtgtagcatatccagaatatccaggccttcatccggaagaggaggtggctaggctctctttcagaaacctgaacactttcttggcagctgatccccgtgtcgagatatcccaggtctctatcggcgacggagtgactatttgccgacgtctttattga

SEQ ID NO: 17 - protein, 239 aa, Narcissus spp. norbelladine
MGASIDDYSLVHKNILHSEDLLKYILETSAYPREHEQLKGLREVTEKHEWSSALVPADEGLFLSMLLKLMNAKRTIEIGVYTGYSLLTTALALPEDGKITAIDVNKSFFEIGLPFIQKAGVEHKINFIESEALPVLDQMLQETKEEDLYDYAFVDADKSNYANYHERLVKLVRIGGAILYDNTLWYGSVAYPEYPGLHPEEEVARLSFRNLNTFLAADPRVEISQVSIGDGVTICRRLY

SEQ ID NO: 18 - DNA, 720 bp, Narcissus spp. norbelladine
  misc_feature (705)..(705): n is a, c, g, or t
atgggtgctagcatagatgattattccctagtccacaagaacatcttgcatagtgaagatcttctgaagtacatattggagactagtgcataccctagagagcatgaacagctcaaggggctgagggaggtcactgagaaacatgaatggagttcagcgcttgtcccagccgatgaaggattgtttctttctatgttgttaaagctcatgaatgctaagagaaccattgagattggtgtgtacaccggctattctcttctcacaactgctttggctttaccggaagatggaaagataacggcaatcgatgtcaacaagtccttctttgagataggactaccatttattcagaaagcaggagttgagcataagatcaatttcattgaatcagaggcacttcctgttcttgatcaaatgcttcaagagatgaaggaagaagacctgtacgactatgcatttgtcgatgcagacaaatctaactatgctaattaccacgagcgattagtgaagctcgtcaggattggaggggcgatcctctacgacaacacgctctggtacggaagtgtagcatatccagaatatccaggccttcatccggaagaggaggtggctaggctctctttcagaaacctgaacactttcttggcagctgatccccgtgtcgagatatcccaggtctctatcggcgacggagtgactatttgncgacgtctttattga

SEQ ID NO: 19 - protein, 239 aa, Narcissus spp. norbelladine
  misc_feature (235)..(235): Xaa can be any naturally occurring amino acid
MGASIDDYSLVHKNILHSEDLLKYILETSAYPREHEQLKGLREVTEKHEWSSALVPADEGLFLSMLLKLMNAKRTIEIGVYTGYSLLTTALALPEDGKITAIDVNKSFFEIGLPFIQKAGVEHKINFIESEALPVLDQMLQEMKEEDLYDYAFVDADKSNYANYHERLVKLVRIGGAILYDNTLWYGSVAYPEYPGLHPEEEVARLSFRNLNTFLAADPRVEISQVSIGDGVTIXRRLY

SEQ ID NO: 20 - DNA, 720 bp, Narcissus spp. norbelladine
atgggtgctagcatagatgattattgcctaattcacaagaaaatcttgcatagtgaagatcttctgaagtacatattggagactagtgcataccccagagagcatgaacagctcaaggggctgagggaggtgactgagaaacatgaatggagttcggcgcttgtcccagccgatgaaggattgtttctttctatgttgataaagctcatgaatgctaagagaaccattgagattggtgtgtacaccggctattctcttctcacaactgctttggctttaccggaagatggaaagataacggcaatcgatgtcaacaagtccttctttgagataggactaccatttattcagaaagcaggagttgagcataagataaatttcattgaatcagaggcacttcctgttcttgatcaaatgcttcaagagacgaaggaagaagacctgtacgactatgcatttgtcgatgcagacaaatctaactacgctaattaccacgagcgattagtgaagctcgtcaggattggaggggcgatcctctacgacaacacgctctggtacggaagtgtagcatatccagaatatccaggccttcatccggaagaggaggtggctaggctctctttcagaaacctgaacactttcttggcagctgatccccgtgtcgagatatcccaggtctctatcggcgacggagtgactatttgccgacgtctttattga

SEQ ID NO: 21 - protein, 239 aa, Narcissus spp. norbelladine
MGASIDDYCLIHKKILHSEDLLKYILETSAYPREHEQLKGLREVTEKHEWSSALVPADEGLFLSMLIKLMNAKRTIEIGVYTGYSLLTTALALPEDGKITAIDVNKSFFEIGLPFIQKAGVEHKINFIESEALPVLDQMLQETKEEDLYDYAFVDADKSNYANYHERLVKLVRIGGAILYDNTLWYGSVAYPEYPGLHPEEEVARLSFRNLNTFLAADPRVEISQVSIGDGVTICRRLY

SEQ ID NO: 22 - DNA, 720 bp, Narcissus spp. norbelladine
atgggtgctagcatagatgattattgcctaattcacaagaaaatcttgcatagtgaagatcttctgaagtacatattggagactagtgcataccccagagagcatgaacagctcaaggggctgagggaggtgactgagaaacatgaatggagttcggcgcttgtcccagccgatgaaggattgtttctttctatgttgttaaagctcatgaatgctaagagaaccattgagattggtgtgtacaccggctattctctgctcacaactgctttggctttaccggaagatggaaagataacggcaatcgatgtcaacaagtccttctttgagataggactaccatttattcagaaagcaggagttgagcataagataaatttcattgaatcagaggcacttcctgttcttgatcaaatgcttcaagagacgaaggaagaagacctgtacgactatgcatttgtcgatgcagacaaatctaactacgctaattaccacgagcgattagtgaagctcgtcaggattggaggggcgatcctctacgacaacacgctctggtacggaagtgtagcatatccagaatatccaggccttcatccggaagaggaggtggctaggctctctttcagaaacctgaacactttcttggcagctgatccccgtgtcgagatatcccaggtctctatcggcgacggagtgactatttgccgacgtctttattga

SEQ ID NO: 23 - protein, 239 aa, Narcissus spp. norbelladine
MGASIDDYCLIHKKILHSEDLLKYILETSAYPREHEQLKGLREVTEKHEWSSALVPADEGLFLSMLLKLMNAKRTIEIGVYTGYSLLTTALALPEDGKITAIDVNKSFFEIGLPFIQKAGVEHKINFIESEALPVLDQMLQETKEEDLYDYAFVDADKSNYANYHERLVKLVRIGGAILYDNTLWYGSVAYPEYPGLHPEEEVARLSFRNLNTFLAADPRVEISQVSIGDGVTICRRLY

SEQ ID NO: 24 - DNA, 241 bp, Narcissus sp. aff. pseudonarcissus
tgttcttgatcaaatgcttgaagagatgaaggaagaagacctgtacgaccatgcatttgtcgatgcagacaaatctaactatgctaattaccacgagcgattagtgaagctcgtcagggttggaggagcgatcctctacgacaacacgctctggaacggaagtgtagcatatccagaatatccaggccttcgtccggaagatgaggtggataggctctctttcagaaacctgaacactttc

SEQ ID NO: 25 - protein, 80 aa, Narcissus sp. aff. pseudonarcissus
VLDQMLEEMKEEDLYDHAFVDADKSNYANYHERLVKLVRVGGAILYDNTLWNGSVAYPEYPGLRPEDEVDRLSFRNLNTF

SEQ ID NO: 26 - DNA, 1542 bp, Narcissus spp. norbelladine
atggccacttcttcttcagcatggctaatgttttcagatcactaccctgaaattctcatcgcaattgcttgcttcctaatattctcattgctgctctccgctcgttcttcgagcgaagacagcctcccttacaactggccgatcttcggaatgcttcctgccatcatttccaacaaccaattcaacgatttcaccactgcccgccttcgcaagatgggctggactttcatcttcaagggcccgtggctcctcgacatggactacattttcacctgtgacccatccaacatcaaccacatgttcaacgacaactttgagaattatccaaaaggtgaacttggaaaggtgttcgatatattcggcaataacatcttcaatgccgacggagatctatggcacgaccatcggaaaatggcgcaaaccatcttgtgggatgggaattaccgcaccatgcaagctactttcatccgaaacaagatggacaatgcgctgattccgattcttgattcagcagcaagcaagcgaaaaccagttgacctgcaagatgtgttttttaggttcacgtttgatacgagttgttttagcgtgctcgctgctgaccccgagtcgctcaccatggaattcccagccgtaccattttcgaaagccgctgatcaagctttggatgcggcactcacacgccatatcacgccgcgcctgatttggaagctgaagagattcttcaacgtcggaagcgagaggacgctggctgtggcgtggaaagtgatcgactcctacatctacgacaagatcgcagagctcaaagcgaaaagaaaactcgtgggaaagatcaatagctatgatgccgtgtctttctacatggataatttcaacatccatgacgataaattccttagagacaatgccttcacgtacttgttagctcagagaaacacgcaatcgctaaccatgacttggttgttctatgcgctatttgaaaacccaaaagtggaactgaaaatcctcagcgagttaaagtcgatcgtcgatgaatcctcagaaagaaaatttaacgatggattcgcattattcgatagcaatatgattcaatctgcaatctatttgcatgcaaccttatgtgaagctctgagaatttatccaccagttccatttgagataaaagatgcacacaaagctgacgttctaccgagtggtcacaaggtcagagctggagaaaagattttattctccccatatgctatggcgaggatgaaggggatctggggagacgactgcttggaattcaagcctgagagatggattactgggaatggaacgctgaagcatgaacccgcttacaagttttttgctttcagtgcaggaccgaggatttgtctgggaaaagagctgtccttcactcagatgaaaatggtcgtcgcgaccatcatatacaatttccatcttcagatggtgaagggacatgtcgtggagcagagtaactccattctcatggacatgaagcatgggctgatggttcaggttagaaagagatcagtcatgtga

SEQ ID NO: 27 - DNA, 1542 bp, Narcissus spp. norbelladine
atggccacttcttcttcagcatggctaatgttttcagatcactaccctgaaattctcatcgcaattgcttgcttcctaatattctcattgctgctctccgctcgttcttcgagcgaagacagcctcccttacaactggccgatcttcggaatgcttcctgccatcatttccaacaaccaattcaacgatttcaccactgcccgccttcgcaagatgggctggactttcatcttcaagggcccgtggctcctcgacatggactacattttcacctgtgacccatctaacatcaaccacatgttcaacgacaacttcgagaattatccaaaaggtgaacttggaaaggtgttcgatatattcggcaataacatcttcaatgccgacggagatctatggcacgaccatcggaaaatggcgcaaaccatcttgtgggatgggaattaccgcaccatgcaagctactttcatccgaaacaagatggacaatgcgctgattccgattcttgattcagcagcctgcaagcgaaaaccagtggacctgcaagatgtgcttcttaggttcacgtttgatacgagttgttttagcgtgctcgctgctgaccccgagtcgctcaccatggaattcccacccgtaccattttcgaaagccgctgatcaagctttggatgcggcactcacacgccatatcacgccgcgcctgatttggaagctcaagagattcttcaacgtcggaagcgagaggacgctggctgtggcgtggaaagtgatcgactcctacatctacgacaagatcgcagagctcaaagcgaaaagaaaactcgtgggaaagatcaatagctacgatgccgtgtctttctacatggataatttcaacatccatgacgataaattccttagagacaatgcgttcacgtacttgttagcacagagaaacacgcaatcgctaaccatgacttggttgttctatgcgctatttgaaaacccaaaagtggaactgaaaatcctcagcgagttgaagtcaatcgtcgatgaatcctcagaaagaaaatttaacgatggattcacattattcgatagcaatatgatacaatctgcaatctatttgcatgcagccttatgtgaagctctgagaatttatccaccagttccatttgagataaaagatgcacacaaagctgacgttctaccgagtggtcacaaggtcagagctggagaaaagattttattctccccatatgctatggcgaggatgaaggggatctggggagacgactgcttggaattcaagcctgagagatggattactgggaatggaacgctgaagcatgaacccgcttacaagttttttgctttcagtgcaggaccgaggatttgtctgggaaaagagctgtccttcactcagatgaaaatggtcgtcgcgaccatcatatacaatttccatcttcagatggtgaagggacatgtcgtggagcagagtaactccattctcatggacatgaagcatgggctgatggttcaggttagaaagagatcagtcatgtga

SEQ ID NO: 28 - DNA, 1542 bp, Narcissus spp. norbelladine
atggccacttcttcttcagcatggctaatcttttcagatcactaccctgaaattctcatcgcaattgcttgcttcctaatattctcattgctgctctccgctcgttcttcgagcgaagacagcctcccttacaactggccaatcttcggaatgcttcctgccatcatttccaacagccaattcaacgatttcaccactgcccgccttcgcaagatgggctggactttcatcttcaagggtccgtggctcctcgacatggactacattttcacctgtgacccatccaacatcaaccacatgttcaacgacaacttcgagaattatccaaaaggcgaacttggtaaggtgttcgatatattcggcaataacatcttcaatgccgacggagatctatggcacgaccatcggaaaatggcccaaaccatcttgtgggatgggaattaccgcaccatgcaagctacgttcatacgaaacaagatggataatgcgctgattccgattcttgattcagcagcaagcaagcgaaaaccagttgacctgcaagatgtgttttttaggttcacgtttgatacgagttgttttagcgtgctcgctgctgaccccgagtcgctcaccatggaattcccagccgtaccattttcgaaagccgctgatcaagctttggatgcggcactcacacgccatatcacgccgcgcctgatttggaagctgaagagattcttcaacgtcggaagcgagaggacgctggctgtggcgtggaaagtgatcgactcctacatctacgacaagatcgcagagctcaaagcgaaaagaaaactcgtgggaaagatcaatagctatgatgccgtgtctttctacatggataatttcaacatccatgacgataaattccttagagacaatgccttcacgtacttgttagctcagagaaacacgcaatcgctaacgatggcttggttattctatgcgctatttgaaaacccaaaagtggaactgaaaatcctcagcgagttaaagtctatcgtcgatgaatcctcagaaagaagatttaacgatggatttgcattattcgatagcaatatgattcaatctgcgatctatttgcatgcaaccttatgtgaagctctgagaatttatccaccagttccatttgagataaaagatgcacacaaagctgacgttctaccgagtggtcacaaggtcagagctggagaaaagattttattctccccatatgctatggcgaggatgaaggggatctggggagacgactgcttggaattcaagcctgagagatggattactgggaatggaacgctgaagcatgaacccgcttacaagttttttgctttcagtgcaggaccgaggatttgtctgggaaaagagctgtccttcactcagatgaaaatggtcgtcgcgaccatcatatacaatttccatcttcagatggtgaagggacatgttgtggagcagagtaactccattctcatggacatgaagcatgggctgatggttcaggttagaaagagatcagtcatgtga

SEQ ID NO: 29 - DNA, 816 bp, Narcissus spp. norbelladine
atggctagcatgactggtggacagcaaatgggtcgcggatccatgtcgttggagaagagatggtctctcgagggaaccacagctttggtcactggaggcaccaaaggaattgggcatgccattgttgaagagttggttgggtttggagccagagtgtacacatgttccaggaatgaagcagagctccgcaaatgtttgcaagagtgggagaacttaaaatatgacgtcacagggtcagtctgcgatgtatcttctcgaaccgaacgagaaaagctagcggaggaggtctcctcagtgttcaatgggaagctgaacatactaatcaataatgcagggggttacgtaaacaagccaattgatggtttcactgccgaggacttctcattcctcgtggctgtcaatttggaatcggctttccatttgtgccagctagcacaccccatgcttaaggcatcgggcacgggcagcatcgtgcacatctcttcttgttgtgcacaaatagctataccagggcacagcatttattcatcaactaaaggagcaataaatcagcttacaaggaatttggcttgtgagtgggcaaaagataacatccgaaccaactccatagctccgggagccataagaacgcctggcacagagtcgtttgtgatcgataaggatgcattggatagagaggtctctcgtgttccattcgggcgcatcggggagccagaggaggtggcatcccttgccgcgttcctctgcatgccatctgcttcctacatcaccggacaagttatatgcgtcgatggaggacggaccataaatggttga

SEQ ID NO: 30 - protein, 513 aa, Narcissus spp. norbelladine
MATSSSAWLMFSDHYPEILIAIACFLIFSLLLSARSSSEDSLPYNWPIFGMLPAIISNNQFNDFTTARLRKMGWTFIFKGPWLLDMDYIFTCDPSNINHMFNDNFENYPKGELGKVFDIFGNNIFNADGDLWHDHRKMAQTILWDGNYRTMQATFIRNKMDNALIPILDSAASKRKPVDLQDVFFRFTFDTSCFSVLAADPESLTMEFPAVPFSKAADQALDAALTRHITPRLIWKLKRFFNVGSERTLAVAWKVIDSYIYDKIAELKAKRKLVGKINSYDAVSFYMDNFNIHDDKFLRDNAFTYLLAQRNTQSLTMTWLFYALFENPKVELKILSELKSIVDESSERKFNDGFALFDSNMIQSAIYLHATLCEALRIYPPVPFEIKDAHKADVLPSGHKVRAGEKILFSPYAMARMKGIWGDDCLEFKPERWITGNGTLKHEPAYKFFAFSAGPRICLGKELSFTQMKMVVATIIYNFHLQMVKGHVVEQSNSILMDMKHGLMVQVRKRSVM

SEQ ID NO: 31 - protein, 513 aa, Narcissus spp. norbelladine
MATSSSAWLMFSDHYPEILIAIACFLIFSLLLSARSSSEDSLPYNWPIFGMLPAIISNNQFNDFTTARLRKMGWTFIFKGPWLLDMDYIFTCDPSNINHMFNDNFENYPKGELGKVFDIFGNNIFNADGDLWHDHRKMAQTILWDGNYRTMQATFIRNKMDNALIPILDSAACKRKPVDLQDVLLRFTFDTSCFSVLAADPESLTMEFPPVPFSKAADQALDAALTRHITPRLIWKLKRFFNVGSERTLAVAWKVIDSYIYDKIAELKAKRKLVGKINSYDAVSFYMDNFNIHDDKFLRDNAFTYLLAQRNTQSLTMTWLFYALFENPKVELKILSELKSIVDESSERKFNDGFTLFDSNMIQSAIYLHAALCEALRIYPPVPFEIKDAHKADVLPSGHKVRAGEKILFSPYAMARMKGIWGDDCLEFKPERWITGNGTLKHEPAYKFFAFSAGPRICLGKELSFTQMKMVVATIIYNFHLQMVKGHVVEQSNSILMDMKHGLMVQVRKRSVM

SEQ ID NO: 32 - protein, 513 aa, Narcissus spp. norbelladine
MATSSSAWLIFSDHYPEILIAIACFLIFSLLLSARSSSEDSLPYNWPIFGMLPAIISNSQFNDFTTARLRKMGWTFIFKGPWLLDMDYIFTCDPSNINHMFNDNFENYPKGELGKVFDIFGNNIFNADGDLWHDHRKMAQTILWDGNYRTMQATFIRNKMDNALIPILDSAASKRKPVDLQDVFFRFTFDTSCFSVLAADPESLTMEFPAVPFSKAADQALDAALTRHITPRLIWKLKRFFNVGSERTLAVAWKVIDSYIYDKIAELKAKRKLVGKINSYDAVSFYMDNFNIHDDKFLRDNAFTYLLAQRNTQSLTMAWLFYALFENPKVELKILSELKSIVDESSERRFNDGFALFDSNMIQSAIYLHATLCEALRIYPPVPFEIKDAHKADVLPSGHKVRAGEKILFSPYAMARMKGIWGDDCLEFKPERWITGNGTLKHEPAYKFFAFSAGPRICLGKELSFTQMKMVVATIIYNFHLQMVKGHVVEQSNSILMDMKHGLMVQVRKRSVM

SEQ ID NO: 33 - protein, 271 aa, Narcissus spp. norbelladine
MASMTGGQQMGRGSMSLEKRWSLEGTTALVTGGTKGIGHAIVEELVGFGARVYTCSRNEAELRKCLQEWENLKYDVTGSVCDVSSRTEREKLAEEVSSVFNGKLNILINNAGGYVNKPIDGFTAEDFSFLVAVNLESAFHLCQLAHPMLKASGTGSIVHISSCCAQIAIPGHSIYSSTKGAINQLTRNLACEWAKDNIRTNSIAPGAIRTPGTESFVIDKDALDREVSRVPFGRIGEPEEVASLAAFLCMPSASYITGQVICVDGGRTING

SEQ ID NO: 34 - DNA, 1729 bp, Arabidopsis thaliana
atctatactcataaatccttttgtctaaaaatggcgatgctaggtttttacgtaacgttcattttctttcttgtatgcctatttacttatttcttcctccaaaagaaacctcaaggtcagcctattctcaagaactggccgttcctcaggatgcttccaggaatgctccaccaaatccctcgtatctacgactggaccgtcgaggtgcttgaggcgaccaatctaactttttatttcaaagggccatggcttagtggaacggacatgttgttcaccgccgatccaaggaatattcatcacatactaagctcaaactttgggaattaccctaaaggacctgagttcaagaagatctttgatgttttgggagaaggaatcttaaccgttgattttgagttgtgggaggagatgaggaagtcaaatcacgccctattccacaatcaagatttcatcgagctctcagtaagtagcaataaaagtaagttaaaagaaggtcttgttccttttcttgataatgctgctcagaaaaacattatcatagaattacaagatgtgttccagagattcatgtttgatacttcttcaattttgatgactggttacgatccaatgtcactatccatcgaaatgctggaagttgagttcggtgaagctgcggatattggcgaagaagcaatctattatagacatttcaaaccggtgatcttgtggaggcttcaaaactggattggtattgggcttgagaggaagatgagaacagctttggccactgtcaatcgtatgtttgcgaagatcatatcttcaagaagaaaagaggagataagtcgcgccaaaacggagccatattccaaggacgcgttgacgtattatatgaatgtggacacgagcaaatataagctcttgaaacctaataaagataagtttataagagatgttatttttagtctagtgttagcaggaagggacaccacaagctcagttctcacttggttcttttggcttctttctaagcatcctcaagttatggccaagctcagacatgagatcaacacaaagtttgataatgaagatctagagaagctcgtgtatctgcatgctgcattgtccgaatcaatgagactctacccgccacttcccttcaaccacaagtctcctgcgaagccagatgtacttccaagcgggcacaaagttgatgcaaattcaaagatcgtgatatgtatctatgcattggggaggatgagatctgtatggggagaagacgcattggatttcaaaccagagagatggatttcagacaatggaggtctaagacatgaaccttcatacaagttcatggcttttaattctggtccgagaacttgcttgggtaaaaatctagctctcttgcagatgaagatggtagctctggagatcatacgaaactatgactttaaggtcattgaaggtcacaaggtcgaaccaattccttctatccttctccgtatgaaacatggtcttaaagtcacagtcacaaagaagatatgattattatgcttgcttggcttctacggcaactattactatttccttatttaaatgtgttacttactagtttgttcccacgttataactacttgtattacgtactaagtacggtgtttgtcccacgtcatgctcataaattaattaatatcgtcaataaagtattagagcatcctcgtccatataaataaaaaggtgttagtggatt

SEQ ID NO: 35 - DNA, 851 bp, Narcissus spp. norbelladine
cgctaatataatccaaacattcaaagaacatgaaagtaaagacaatcgaataagttcaacacataccaactaaattcttaacatacctaagggtacaaataggacatctgctgatataagaatacatgaactcccttccattacaaatgtgaagaccatcaatgtgatcaccaccatgtttattagagacatacctccccatgataatactagcacaggagttaataatacgagcattcggagttcataatactagtagcatgggagtctcacatgactgatctctttctaacctgaaccatcagcccatgcttcatgtccatgagaatggagttactctgctccacgacatgtcccttcaccatctgaagatggaaattgtatatgatggtcgcgacgaccattttcatctgagtgaaggacagctcttttcccagacaaatcctcggtcctgcactgaaagcaaaaaacttgtaagcgggttcatgcttcagcgttccattcccagtaatccatctctcaggcttgaattccaagcagtcgtctccccagatccccttcatcctcgccatagcatatggggagaataaaatcttttctccagctctgaccttgtgaccactcggtagaacgtcagctttgtgtgcatcttttatctcaaatggaactggtggataaattctcagagcttcacataaggttgcatgcaaatagattgcagattgaatcatattgctatcgaataatgcgaatccatcgttaaattttctttctgaggattcatcgacgatcgactttaactcgctgaggattttcagttccacttttgggttttcaaatagcgcatagaa

(19) United States
(12) Patent Application Publication
(10) Pub. No.: US 2019/0300892 A1
(43) Pub. Date: Oct. 3, 2019

(54) CONSTRUCTS AND METHODS FOR BIOSYNTHESIS OF GALANTHAMINE

(71) Applicant: Donald Danforth Plant Science Center, St. Louis, MO (US)

(72) Inventors: Toni M Kutchan, St. Louis, MO (US); Matthew Kilgore, St. Louis, MO (US)

(21) Appl. No.: 15/384,160

(22) Filed: Dec. 19, 2016

Related U.S. Application Data
(63) Continuation of application No. PCT/US2015/036737, filed on Jun. 19, 2015.
(60) Provisional application No. 62/014,971, filed on Jun. 20, 2014.

(51) Int. Cl. C12N 15/82; C12N 15/52; C12N 9/10; C12Q 1/6895

(52) U.S. Cl. CPC C12N 15/8243; C12Q 1/6895; C12N 9/1007; C12N 15/52

(57) ABSTRACT

The present disclosure relates generally to the identification of biosynthetic pathway genes. In particular, it relates to the identification of enzymes within the Amaryllidaceae alkaloid biosynthetic pathway as well as to engineering transgenic organisms for the production of galanthamine and/or hemanthamine and/or lycorine.

### CROSS REFERENCE TO RELATED APPLICATIONS

The present application claims priority to U.S. Provisional Application No. 62/014,971, filed Jun. 20, 2014, entitled “Constructs and Methods for Biosynthesis of Galanthamine,” and International Application No. WO 2015/196100 entitled “Constructs and Methods for Biosynthesis of Galanthamine,” and is herein incorporated by reference.

### STATEMENT REGARDING FEDERALLY SPONSORED RESEARCH

This invention was made with government support under 1RC2GM092561(NIGMS) awarded by the National Institutes of Health. The government has certain rights in the invention.

### INCORPORATION BY REFERENCE OF THE SEQUENCE LISTING

The accompanying “Sequence Listing” forms a part of this application and the sequences disclosed therein are herein incorporated by reference.

### BACKGROUND

The discovery of genes involved in metabolism is essential to metabolic engineering and synthetic biology. The elucidation of plant biochemical pathways can take decades. In fact, the biosynthesis of morphine, an important opiate analgesic, is still not completely elucidated at the gene level, even though the first enzyme specific to morphine biosynthesis was discovered more than 20 years ago in 1993. Reports on the enzymatic activities of poppy extracts to describe the morphine biosynthetic pathway go even farther back to 1971. After more than 40 years of enzymology and reverse genetics, the morphine biosynthetic pathway is still incomplete at the gene level. Traditionally, plant biochemical pathway enzymes have been identified either directly by purification from plant extracts or indirectly by examining enriched cDNA libraries and functionally expressing clones. To reduce pathway discovery from a 20+ year process to a more reasonable time frame, new methods must be developed and embraced.

Amaryllidaceae alkaloids are a group of alkaloids with many documented biological activities. This makes them valuable potential medicines several examples are the anti-cancer compounds hemanthamine and lycorine and the anti-viral compound pancratistatin. One example of an Amaryllidaceae alkaloid already used medically to treat Alzheimer's disease is galanthamine. Galanthamine, also known in the literature as galantamine, is an alkaloid, discovered in 1953, produced by members of the Amaryllidaceae family. It reduces the symptoms of Alzheimer's disease through acetylcholine esterase inhibition and nicotinic receptor binding. These activities are thought to compensate for reduced acetylcholine sensitivity in Alzheimer's disease by increasing acetylcholine levels and perhaps increasing acetylcholine sensitivity. Until now, no committed galanthamine biosynthetic genes have been identified. Limited enzyme kinetic characterization has been done on plant protein extracts enriched for the norbelladine 4′-O-methyltransferase (N4OMT) of Nerine bowdenii, but the underlying gene was never identified.

The current understanding of the biosynthesis of galanthamine is based on radiolabeling experiments. Work on other Amaryllidaceae alkaloids including lycorine and hemanthamine studying steps prior to 4′-O-methylnorbelladine can be applied to galanthamine biosynthesis because 4′-O-methylnorbelladine is a universal substrate for these alkaloids. The pathway starts with the amino acid substrates phenylalanine and tyrosine. In Narcissus incomparabilis phenylalanine was established as a precursor that contributes the catechol portion of norbelladine. This was done using radiolabeling experiments to trace incorporation of [3-¹⁴C]phenylalanine into lycorine and degradation experiments on the resulting lycorine to determine the location of the ¹⁴C label. Similar experiments with phenylalanine were performed in Nerine bowdenii monitoring hemanthamine incorporation. As a follow up radiolabeling experiments were used to determine that phenylalanine probably proceeds sequentially through the intermediates trans-cinnamic acid, p-hydroxycinnamic acid and 3,4-dihydroxycinnamic acid or p-hydroxybenzaldehyde before conversion into 3,4-dihydroxybenzaldehyde. Tyrosine has been established as a precursor of galanthamine that in contrast to phenylalanine contributes only to the non-catechol half of the norbelladine intermediate. This was done by observing [2-¹⁴C]tyrosine incorporation into galanthamine and degradation experiments of galanthamine. Tyrosine decarboxylase converts tyrosine into tyramine and is well characterized in other plant families. 3,4-Dihydroxybenzaldehyde and tyramine condense into a Schiff-base and are reduced to form the first alkaloid in the proposed pathway, norbelladine. Norbelladine has been documented to incorporate into galanthamine and all major Amaryllidaceae alkaloid types in ¹⁴C radiolabeling studies. 4′-O-methylnorbelladine is then formed by O-methylation of norbelladine. A phenol-coupling reaction, followed by spontaneous oxide bridge formation, creates N-demethylnarwedine, which is then reduced and N-methylated to yield galanthamine (FIGS. 1 and 12). In one study, Barton et al. fed O-methyl[1-¹⁴C]norbelladine to flower stalks of King Alfred daffodils, but it was not incorporated into galanthamine. The authors concluded that the intermediate in the pathway must be 4′-O-methyl-N-methylnorbelladine despite low incorporation of this compound when the equivalent experiment was conducted with 4′-O-methyl-[N-methyl-¹⁴C]norbelladine. A recent revision of the proposed pathway by Eichhorn et al. contradicted this conclusion and placed the N-methylation step at the end of the proposed pathway instead of before the phenol-coupling reaction. In that study, [OC³H₃]4′-O-methylnorbelladine was applied to ovary walls of Leucojum aestivum. Incorporation into products indicated that the pathway produced N-demethylated intermediates up until the penultimate step to galanthamine. N-methylation was proposed as the final step of biosynthesis. The use of galanthamine or an analogue or a pharmaceutically acceptable acid addition salt thereof for the preparation of a medicament for treating Alzheimer's Dementia (AD) and related dementias has been described in EP 0,236,684 (U.S. Pat. No. 4,663,318).

The use of galanthamine for treating alcoholism and the administration via a transdermal therapeutic system (TTS) or patch is disclosed in EP 0,449,247 and WO 94/16707. Similarly, the use of galanthamine in the treatment of nicotine dependence using administration via a transdermal therapeutic system (TTS) or patch is disclosed in WO 94/16708. Treatment of nerve gas poisoning is disclosed in DE 4,342,174.

A number of applications by disclose the use of galanthamine, analogues thereof and pharmaceutically acceptable salts thereof for the preparation of medicaments for treating mania (U.S. Pat. No. 5,336,675), chronic fatigue syndrome (CFS) (EP 0,515,302; U.S. Pat. No. 5,312,817), the negative effects of benzodiazepine treatment (EP 0,515,301) and the treatment of schizophrenia (U.S. Pat. No. 5,633,238). In these applications and patents, e.g. in U.S. Pat. No. 5,312,817, a number of immediate release tablet formulations of galanthamine hydrobromide are given.

Galanthamine and companion alkaloids are usually isolated from plants belonging to the Amaryllidaceae family, for example Galanthus species and Leucojum aestivum, although the quantity that can be isolated varies greatly across the family. Some species of these plants have galanthamine in concentrations of up to 0.3% with only small amounts of companion alkaloids so that the extraction method described in DE-PS 11 93 061 can be used. This process of extraction is not feasible to practice at an industrial scale. Apart from plant sources, a chemical process for the synthesis of galanthamine and its analogues including its acid addition salts, has been disclosed in WO 95/27715.

At present, galanthamine is produced for commercial purposes through wild collection of Galanthus species, and certain species of daffodil. These species are scarce, and isolation of galanthamine from daffodil is expensive. A 1996 figure placed the cost of isolation of galanthamine from daffodil at $50,000 U.S. dollars per kilogram, with a yield of only 0.1-0.2% dry weight.

While synthetic methods for the preparation of galanthamine are available, they are complicated and expensive, and a more economic, sustainable “green” source of this pharmaceutical is highly desirable.

### SUMMARY

Accordingly, to meet this need in the art, disclosed herein is the isolation and characterization of cDNAs and encoded norbelladine 4′-O-methyltransferase, CYP96T1-3, and norbelladine synthase/reductase involved in the biosynthesis of galanthamine and haemanthamine. These cDNAs can be used to develop a synthetic biological source of galanthamine by building the galanthamine biosynthetic pathway into plants. Camelina will be used as a model system to demonstrate proof of concept. Other plants useful in the practice of the present methods, include but are not limited to: species of Galanthus, species of Brachypodium, species of Setaria, species of Populus, tobacco, corn, rice, soybean, cassava, canola (rapeseed), wheat, peanut, palm, coconut, safflower, sesame, cottonseed, sunflower, flax, olive, safflower, sugarcane, castor bean, switchgrass, Miscanthus, Camelina and Jatropha. Other plants useful in the practice of the present methods may include plants from the family Amaryllidaceae including but not limited to daffodil, Narcissus spp.; snowdrop, Galanthus nivalis; and summer snowflake, Leucojum aestivum.

The galanthamine biosynthetic pathway is an ideal candidate for developing a gene discovery pipeline because while there is a detailed knowledge of intermediates in the pathway, there is limited knowledge of its enzymology. The previous work on galanthamine biosynthesis makes the prediction of enzyme classes involved in the proposed pathway possible, thereby rendering the galanthamine pathway a suitable system for development of an omic methodology for biochemical pathway discovery.

Besides describing the engineering of the galanthamine biosynthetic pathway into plants, the results presented below more broadly provide proof-of-concept of a novel workflow designed to streamline the identification of biosynthetic pathway genes. As demonstrated in the example below, a de novo transcriptome is created for Narcissus sp. aff. pseudonarcissus using illumina sequencing. HAYSTACK, a program that utilizes the Pearson correlation, is used to find genes that co-express with galanthamine accumulation in this transcriptome. This set of candidates is interrogated for homologs to methyltransferases. An OMT that converts norbelladine to 4′-O-methylnorbelladine (NpN4OMT) in the proposed biosynthesis of galanthamine is identified in this manner and characterized. A cytochrome P450 and norbelladine synthase/reductase are also identified using HAYSTACK to find transcripts that co-express with N4OMT in Narcissus sp. aff. pseudonarcissus, Galanthus sp. and Galanthus elwesii transcriptomes. Candidates co-expressing with N40MT in the majority of the transcriptomes that were homologues to cytochrome P450s or reductases were characterized. One of these cytochrome P450s, CYP96T1, was found to make the compounds N-demethylnarwedine, (10aS,4bS)-noroxomaritidine and (10aR,4bR)-noroxomaritidine. Also, one reductase was found to be norbelladine synthase/reductase and make norbelladine form a mixture of 3,4-dihydroxybenzaldehyde and tyramine.

Further scope of the applicability of the presently disclosed embodiments will become apparent from the detailed description and drawings provided below. However, it should be understood that the detailed description and specific examples, while indicating preferred embodiments of this disclosure, are given by way of illustration only since various changes and modifications within the spirit and scope of these embodiments will become apparent to those skilled in the art from this detailed description.

### BRIEF DESCRIPTION OF THE DRAWINGS

The foregoing and other aspects, features, and advantages of the present disclosure will be better understood from the following detailed description taken in conjunction with the accompanying figures, all of which are given by way of illustration only, and are not limitative of the present specification, in which:

FIG. 1. Proposed biosynthetic pathway for galanthamine. 3,4-Dihydroxybenzaldehyde derived from phenylalanine and tyramine derived from tyrosine are condensed to form norbelladine. Norbelladine is methylated by NpN4OMT to 4′-O-methylnorbelladine. 4′-O-Methylnorbelladine is oxidized to N-demethylnarwedine. N-demethylnarwedine is then reduced to N-demethylgalanthamine. In the last step, N-demethylgalanthamine is methylated to galanthamine.

FIG. 2. The identification of the candidate NpN4OMT. (A) Venn diagram of all sequences, all OMTs, and all galanthamine correlating sequences according to HAYSTACK. (B) Accumulation level of galanthamine in Narcissus spp. (C) Candidate NpN4OMT expression profile in leaf, bulb and inflorescence with the relative initial read estimate and qRT-PCR ΔΔCt on the y-axis with leaf tissue set to 1.

FIG. 3. Phylogenetic analysis of NpN4OMT1. A maximum-likelihood phylogenetic tree of characterized methyltransferases listed in Table 3. Alignment constructed using MUSCLE.

FIG. 4. NpN4OMT1 purification and enzymatic assay with NMR structure elucidation of the 4′-O-methylorbelladine product. (A) 10% wt/vol SDS-PAGE gel including fractions from crude extract and the desalted isolated protein preparation. This is shown for vector only, NpN4OMT1 and Pfs preparations. (B) Enzyme assays, top to bottom: norbelladine standard, 4′-O-methylnorbelladine standard, assay with E. coli vector only crude extract added, assay without AdoMet added, working methyltransferase assay. (C) NMR structure elucidation; proton chemical shifts are black, carbon chemical shifts are blue, key HMBC correlations are black arrows, and key ROESY correlations are red arrows.

FIG. 5. NpN4OMT product 4′-O-methylnorbelladine proton NMR spectra with peak assignments.

FIG. 6. NpN4OMT product 4′-O-methylnorbelladine COSY spectra.

FIG. 7. NpN4OMT product 4′-O-methylnorbelladine HMBC spectra.

FIG. 8. NpN4OMT product 4′-O-methylnorbelladine ROESY spectra.

FIG. 9. NpN4OMT product 4′-O-methylnorbelladine HSQC spectra.

FIG. 10. The effects of divalent cations, temperature, and pH on the NpN4OMT1. (A) Divalent cations tested with 5 min assays with 5 μM of cation Ca²⁺, Co²⁺, Zn²⁺, Mg²⁺ or Mn²⁺. (B) pH optimum 15 min assays with 5 μM Mg²⁺. (C) Temperature optimum 15 min assays with 5 M Mg²⁺. Divalent cation and pH testing reactions are 100 μl reactions at 37° C. The divalent cation test has 4 μM norbelladine while pH and temperature optimum have 100 μM norbelladine.

FIG. 11. The protein sequence alignment of NpN4OMT variants. 5 unique variations of the NpN4OMT sequence are aligned against the original sequence predicted by the transcriptome using CLC software. Nucleotide sequences: NpN4OMT1 (SEQ ID NO: 14), NpN4OMT2 (SEQ ID NO:16), NpN4OMT3 (SEQ ID NO:18), NpN4OMT4 (SEQ ID NO:20) and NpN4OMT5 (SEQ ID NO:22). Amino Acid sequences: NpN4OMT1 (SEQ ID NO:15), NpN4OMT2 (SEQ ID NO: 17), NpN4OMT3 (SEQ ID NO: 19), NpN4OMT4 (SEQ ID NO:21) and NpN4OMT5 (SEQ ID NO:23). Dots are identical residues.

FIG. 12. Proposed biosynthetic pathways for representative Amaryllidaceae alkaloids directly derived from C—C phenol coupling. The discovered NpN4OMT, CYP96T1, norbelladine synthase/reductase and potential enzyme classes involved in each step of the pathways are in blue.

FIG. 13. Work-flow for identification of candidate cytochrome P450 and norbelladine synthase/reductase enzymes. Following the generation of transcriptome assemblies, cytochrome P450 enzymes and homologues to various reductases were identified with BLASTP (Navy blue) and genes correlating with N4OMT were identified with HAYSTACK (Red). The cytochrome P450 search is diagramed for illustration. The genes present in both lists makeup the initial candidate gene list (Green). Homologues of these genes were identified in the N4OMT correlating lists of the other transcriptomes using BLASTN (Gray). Candidates with homologues in all five N4OMT correlating lists were cloned from daffodil, Narcissus sp. (light blue). The analysis for the daffodil ABySS and MIRA assembly is completely diagramed to illustrate the process followed in every assembly. The number of transcripts selected in each step are in parentheses. The daffodil Trinity assembly is excluded from this work-flow due to its poor quality.

FIG. 14. MUSCLE alignment of protein sequences for CYP96T1, CYP96T2, CYP96T3, the CYP96T1 sequence from the daffodil ABySS and MIRA assembly and CYP96A15 from Arabidopsis thaliana (Q9FVS9). Simplified consensus motifs for cytochrome P450 enzymes are placed above the CYP96T1 sequence. Dots are exact matches to CYP96T1 and dashes are gaps.

FIG. 15. LC-MS/MS enhanced product ion scan (EPI) monitoring the C—C phenol coupling of 4′-O-methylnorbelladine and 4′-O-methyl-N-methylnorbelladine in CYP96T1 assays. Arrows indicate peaks unique to Sf9 cell containing assays with substrate present. (A) Standards and assays with 4′-O-methylnorbelladine as the substrate. Sample runs top to bottom (10aS,4bS)- and (10aR,4bR)-noroxomaritidine standard (1 μM), CYP96T1 assay, CPR assay, CYP96T1 assay without 4′-O-methylnorbelladine and assay without Sf9 cells. (B) Standards and assays with 4′-O-methyl-N-methylnorbelladine as the substrate. Top to bottom narwedine standard, CYP96T1 assay, CPR assay, assay without 4′-O-methylnorbelladine and assay without Sf9 cells. (C) EPI of the (10aS,4bS)- and (10aR,4bR)-noroxomaritidine standard. (D) EPI of the CYP96T1 (10aS,4bS)- and (10aR,4bR)-noroxomaritidine product with 4′-O-methylnorbelladine as substrate. (E) EPI of the CYP96T1 para-para′ product with 4′-O-methyl-N-methylnorbelladine as substrate. Red fragments indicate the addition of one methyl group, 14 m/z, relative to (10aS,4bS)- and (10aR,4bR)-noroxomaritidine and blue fragments indicate the same m/z as (10aS,4bS)- and (10aR,4bR)-noroxomaritidine fragments. Intensity is presented in counts per second (CPS).

FIG. 16. Chromatographic separation and MS/MS analysis of the primary 4′-O-methylnorbelladine products (10aR,4bR)- and (10aS,4bS)-noroxomaritidine The epimers (10aR,4bR)- and (10aS,4bS)-noroxomaritidine were chromatographically separated with a chiral-CBH column and analyzed by MS/MS using an enhanced product ion (EPI) scan. (A) Samples, top to bottom: (10aS,4bS)- and (10aR,4bR)-noroxomaritidine standard, CYP96T1 assay, CPR assay, CYP96T1 assay without 4′-O-methylnorbelladine substrate and no Sf9 cells assay. (B) EPI fragmentation pattern for epimer 1 of (10aS,4bS)- and (10aR,4bR)-noroxomaritidine. (C) EPI fragmentation pattern for epimer 2 of (10aS,4bS)- and (10aR,4bR)-noroxomaritidine. (D) EPI fragmentation pattern for epimer 1 in the CYP96T1 assay with 4′-O-methylnorbelladine as substrate. (E) EPI fragmentation pattern for epimer 2 in the CYP96T1 assay with 4′-O-methylnorbelladine as substrate. Intensity is presented in counts per second (CPS).

FIG. 17. LC-MS/MS Enhanced Product Ion (EPI) scan of sodium borohydride (NaBH₄) treated CYP96T1 assays with 4′-O-methylnorbelladine substrate. (A) Chromatograph with the following sample runs top to bottom: N-demethylgalanthamine standard, CYP96T1 assay, CPR assay, assay with no Sf9 cells and CYP96T1 assay without 4′-O-methylnorbelladine. (B) EPI fragmentation pattern of the N-demethylgalanthamine standard peak eluting at 4 min. (C) EPI fragmentation pattern of the N-demethylgalanthamine product in the CYP96T1 assay. (D) EPI fragmentation pattern of epi-N-demethylgalanthamine from the CYP96T1 assay. (E) EPI fragmentation pattern of (10aR,4bR)- and (10aS,4bS)-noroxomaritidine standard reduced to stereoisomeric 8-O-demethylmaritidine. (F) EPI fragmentation pattern of reduced (10aR,4bR)- and (10aS,4bS)-noroxomaritidine product from CYP96T1 assays.

FIG. 18. Relative product formed in assays with 4′-O-methylnorbelladine (A and B) or 4′-O-methyl-N-methylnorbelladine (C, D and E) as substrate. Assays are performed in triplicate only expressing CPR or with CPR in combination with CYP96T1. (A) para-para′((10aS,4bS)- and (10aR,4bR)-noroxomaritidine) product. (B) para-ortho′(N-demethylnarwedine) product. (C) Potentially para-para′C—C phenol coupling product. (D) para-ortho′(Narwedine) product. (E) Potentially ortho-para′C—C phenol coupling product.

FIG. 19. LC/MS/MS analysis of the norbelladine synthase/reductase assays. Top to bottom norbelladine standard, functioning norbelladine synthase/reductase assay, norbelladine synthase/reductase assay without tyramine and 3,4-dihydroxybenzaldehyde, norbelladine synthase/reductase assay without NADPH, norbelladine synthase/reductase assay with E. coli vector control protein extract but no norbelladine synthase/reductase protein, solvent injection blank.

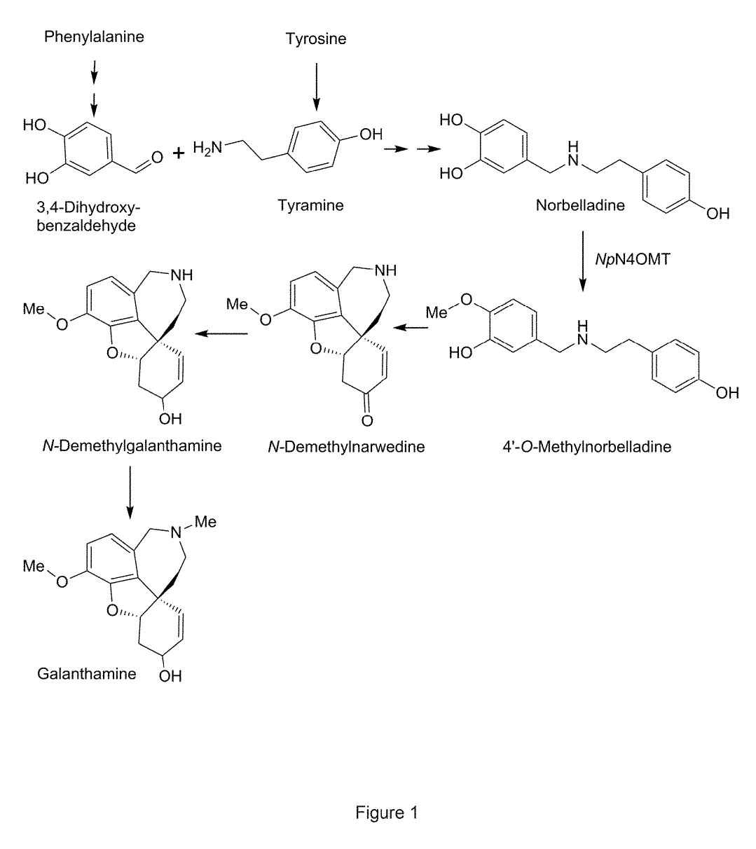

### DESCRIPTION

The following detailed description is provided to aid those skilled in the art. Even so, the following detailed description should not be construed to unduly limit, as modifications and variations in the embodiments discussed herein may be made by those of ordinary skill in the art without departing from the spirit or scope of the present disclosure.

Any feature, or combination of features, described herein is (are) included within the scope of the present disclosure, provided that the features included in any such combination are not mutually inconsistent as will be apparent from the context, this specification, and the knowledge of one of ordinary skill in the art. Additional advantages and aspects of the present disclosure are apparent in the following detailed description and claims.

The contents of each of the publications, patent applications, patents, and other references mentioned herein are incorporated by reference in their entirety. In case of conflict, the present disclosure, including explanations of terms, will control.

### I. Terms

The following definitions are provided to aid the reader in understanding the various aspects of the present disclosure. Unless defined otherwise, all technical and scientific terms used herein have the same meaning as commonly understood by those of ordinary skill in the art to which the disclosure pertains. Units, prefixes and symbols may be denoted in their SI accepted form. Provision, or lack of the provision, of a definition for a particular term or phrase is not meant to signify any particular importance, or lack thereof. Rather, and unless otherwise noted, terms used and the manufacture or laboratory procedures described herein are well known and commonly employed in the art. Conventional methods are used for these procedures, such as those provided in the art and various general references.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “comprising” as used in a claim herein is open-ended, and means that the claim must have all the features specifically recited therein, but that there is no bar on additional features that are not recited being present as well. The term “comprising” leaves the claim open for the inclusion of unspecified ingredients even in major amounts. The term “consisting essentially of” in a claim means that the invention necessarily includes the listed ingredients, and is open to unlisted ingredients that do not materially affect the basic and novel properties of the invention. A “consisting essentially of” claim occupies a middle ground between closed claims that are written in a closed “consisting of” format and fully open claims that are drafted in a “comprising′ format”. These terms can be used interchangeably herein if, and when, this may become necessary. Furthermore, the use of the term “including”, as well as other related forms, such as “includes” and “included”, is not limiting.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art and is understood as included in embodiments where it would be appropriate. Nucleotides may be referred to by their commonly accepted single-letter codes. Unless otherwise indicated, amino acid sequences are written left to right in amino to carboxyl orientation, respectively. Amino acids may be referred to herein by either their commonly known three letter symbols or by the one-letter symbols recommended by the IUPAC-IUM Biochemical Nomenclature Commission. It is further to be understood that all base sizes or amino acid sizes, and all molecular weight or molecular mass values, given for nucleic acids or polypeptides are approximate, and are provided for description purposes and are not to be unduly limiting. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

If ranges are disclosed, the endpoints of all ranges directed to the same component or property are inclusive and independently combinable (e.g., ranges of “up to about 25 wt. %, or, more specifically, about 5 wt. % to about 20 wt. %,” is inclusive of the endpoints and all intermediate values of the ranges of “about 5 wt. % to about 25 wt. %,” etc.). Numeric ranges recited with the specification are inclusive of the numbers defining the range and include each integer within the defined range.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or +a range of up to 20%, up to 15%, up to 10%, up to 5%, or up to 4%, 3%, 2%, or 1% compared to the specifically recited value.

As used herein, “altering level of production” or “altering level of expression” means changing, either by increasing or decreasing, the level of production or expression of a nucleic acid sequence or an amino acid sequence (for example a polypeptide, an siRNA, a miRNA, an mRNA, a gene), as compared to a control level of production or expression.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer (1979) Principles of Protein Structure, Springer-Verlag). According to such analyses, groups of amino acids can be defined where amino acids within a group exchange preferentially with each other, and therefore resemble each other most in their impact on the overall protein structure.

Examples of amino acid groups defined in this manner include: a “charged/polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, Ile, Met, Ser, Thr and Cys. Within each group, subgroups can also be identified, for example, the group of charged/polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala. Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained.

As used herein “control” or “control level” means the level of a molecule, such as a polypeptide or nucleic acid, normally found in nature under a certain condition and/or in a specific genetic background. In certain embodiments, a control level of a molecule can be measured in a cell or specimen that has not been subjected, either directly or indirectly, to a treatment. A control level is also referred to as a wildtype or a basal level. These terms are understood by those of ordinary skill in the art. A control plant, i.e. a plant that does not contain a recombinant DNA that confers (for instance) an enhanced trait in a transgenic plant, is used as a baseline for comparison to identify an enhanced trait in the transgenic plant. A suitable control plant may be a non-transgenic plant of the parental line used to generate a transgenic plant. A control plant may in some cases be a transgenic plant line that comprises an empty vector or marker gene, but does not contain the recombinant DNA, or does not contain all of the recombinant DNAs in the test plant.

The terms “enhance”, “enhanced”, “increase”, or “increased” refer to a statistically significant increase. For the avoidance of doubt, these terms generally refer to about a 5% increase in a given parameter or value, about a 10% increase, about a 15% increase, about a 20% increase, about a 25% increase, about a 30% increase, about a 35% increase, about a 40% increase, about a 45% increase, about a 50% increase, about a 55% increase, about a 60% increase, about a 65% increase, about 70% increase, about a 75% increase, about an 80% increase, about an 85% increase, about a 90% increase, about a 95% increase, about a 100% increase, or more over the control value. These terms also encompass ranges consisting of any lower indicated value to any higher indicated value, for example “from about 5% to about 50%”, etc.

As used herein, “expression” or “expressing” refers to production of a functional product, such as, the generation of an RNA transcript from an introduced construct, an endogenous DNA sequence, or a stably incorporated heterologous DNA sequence. A nucleotide encoding sequence may comprise intervening sequence (e.g. introns) or may lack such intervening non-translated sequences (e.g. as in cDNA). Expressed genes include those that are transcribed into mRNA and then translated into protein and those that are transcribed into RNA but not translated (for example, siRNA, transfer RNA and ribosomal RNA). The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

An “expression cassette” refers to a nucleic acid construct, which when introduced into a host cell, results in transcription and/or translation of a RNA or polypeptide, respectively.

The term “genome” as it applies to a plant cells encompasses not only chromosomal DNA found within the nucleus, but organelle DNA found within subcellular components (e.g., mitochondrial, plastid) of the cell. As used herein, the term “genome” refers to the nuclear genome unless indicated otherwise. However, expression in a plastid genome, e.g., a chloroplast genome, or targeting to a plastid genome such as a chloroplast via the use of a plastid targeting sequence, is also encompassed by the present disclosure.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants. Heterologous nucleic acid fragments, such as coding sequences that have been inserted into a host organism, are not normally found in the genetic complement of the host organism. As used herein, the term “heterologous” also refers to a nucleic acid fragment derived from the same organism, but which is located in a different, e.g., non-native, location within the genome of this organism. Thus, the organism can have more than the usual number of copy(ies) of such nucleic acid fragment located in its(their) normal position within the genome and in addition, in the case of plant cells, within different genomes within a cell, for example in the nuclear genome and within a plastid or mitochondrial genome as well. A nucleic acid fragment that is heterologous with respect to an organism into which it has been inserted or transferred is sometimes referred to as a “transgene.”

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences or homologs. The term “homologous” refers to the relationship between two nucleic acid sequence and/or proteins that possess a “common evolutionary origin”, including nucleic acids and/or proteins from superfamilies (e.g., the immunoglobulin superfamily) in the same species of animal, as well as homologous nucleic acids and/or proteins from different species of animal (for example, myosin light chain polypeptide, etc.; see Reeck et al., (1987) Cell, 50:667). Such proteins (and their encoding nucleic acids) may have sequence homology, as reflected by sequence similarity, whether in terms of percent identity or by the presence of specific residues or motifs and conserved positions. The methods disclosed herein contemplate the use of the presently disclosed nucleic and protein sequences, as well as sequences having sequence identity and/or similarity.

By “host cell” it is meant a cell which contains a vector and supports the replication and/or expression of the vector. Host cells may be prokaryotic cells such as E. coli, or eukaryotic cells such as yeast, insect, amphibian, or mammalian cells. Alternatively, the host cells are monocotyledonous or dicotyledonous plant cells.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. “Introduced” includes reference to stable or transient transformation methods, as well as sexually crossing. Thus, “introduced” in the context of inserting a nucleic acid fragment (e.g., a recombinant DNA construct/expression construct) into a cell, can mean “transfection” or “transformation” or “transduction”, and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

As used herein the term “isolated” refers to a material such as a nucleic acid molecule, polypeptide, or small molecule, such as galanthamine, that has been separated from the environment from which it was obtained. It can also mean altered from the natural state. For example, a polynucleotide or a polypeptide naturally present in a living animal is not “isolated” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as “isolated polypeptides” or “isolated nucleic acid molecules”, etc., are polypeptides or nucleic acid molecules that have been purified, partially or substantially, from a recombinant host cell or from a native source.

As used here “modulate” or “modulating” or “modulation” and the like are used interchangeably to denote either up-regulation or down-regulation of the expression or biosynthesis of a material such as a nucleic acid, protein or small molecule relative to its normal expression or biosynthetic level in a wild type or control organism. Modulation includes expression or biosynthesis that is increased or decreased by about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.5%, 99.9%, 100%, 110%, 115%, 120%, 125%, 130%, 135%, 140%, 145%, 150%, 155%, 160%, 165% or 170% or more relative to the wild type or control expression or biosynthesis level. As described herein, various material accumulation, such as that of galanthamine, can be increased, or in the case of some embodiments, sometimes decreased relative to a control. One of ordinary skill will be able to identify or produce a relevant control.

As used herein, “nucleic acid” means a polynucleotide (or oligonucleotide), including single or double-stranded polymers of deoxyribonucleotide or ribonucleotide bases, and unless otherwise indicated, encompasses naturally occurring and synthetic nucleotide analogues having the essential nature of natural nucleotides in that they hybridize to complementary single-stranded nucleic acids in a manner similar to naturally occurring nucleotides. Nucleic acids may also include fragments and modified nucleotide sequences. Nucleic acids disclosed herein can either be naturally occurring, for example genomic nucleic acids; or isolated, purified, non-genomic nucleic acids, including synthetically produced nucleic acid sequences such as those made by chemical oligonucleotide synthesis, enzymatic synthesis, or by recombinant methods, including for example, cDNA, codon-optimized sequences for efficient expression in different transgenic plants reflecting the pattern of codon usage in such plants, nucleotide sequences that differ from the nucleotide sequences disclosed herein due to the degeneracy of the genetic code but that still encode the protein(s) of interest disclosed herein, nucleotide sequences encoding the presently disclosed protein(s) comprising conservative (or non-conservative) amino acid substitutions that do not adversely affect their normal activity, PCR-amplified nucleotide sequences, and other non-genomic forms of nucleotide sequences familiar to those of ordinary skill in the art.

As used herein, “nucleic acid construct” or “construct” refers to an isolated polynucleotide which can be introduced into a host cell. This construct may comprise any combination of deoxyribonucleotides, ribonucleotides, and/or modified nucleotides. This construct may comprise an expression cassette that can be introduced into and expressed in a host cell.

As used herein “operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

As used herein, the terms “plant” or “plants” that can be used in the present methods broadly include the classes of higher and lower plants amenable to transformation techniques, including angiosperms (monocotyledonous and dicotyledonous plants), gymnosperms, ferns, and unicellular and multicellular algae. The term “plant” also includes plants which have been modified by breeding, mutagenesis or genetic engineering (transgenic and non-transgenic plants). It includes plants of a variety of ploidy levels, including aneuploid, polyploid, diploid, haploid and hemizygous. The plant may be in any form including suspension cultures, embryos, meristematic regions, callus tissue, gametophytes, sporophytes, pollen, microspores, whole plants, shoot vegetative organs/structures (e.g. leaves, stems and tubers), roots, flowers and floral organs/structures, seed (including embryo, endosperm, and seed coat) and fruit, plant tissue (e.g. vascular tissue, ground tissue, and the like) and cells, and progeny of same. The term “food crop plant” includes plants that are either directly edible, or which produce edible products, and that are customarily used to feed humans either directly, or indirectly through animals. Non-limiting examples of such plants include: Cereal crops: wheat, rice, maize (corn), barley, oats, sorghum, rye, and millet; Protein crops: peanuts, chickpeas, lentils, kidney beans, soybeans, lima beans; Roots and tubers: potatoes, sweet potatoes, and cassavas; Oil crops: corn, soybeans, canola (rapeseed), wheat, peanuts, palm, coconuts, safflower, sesame, cottonseed, sunflower, flax, olive, and safflower; Sugar crops: sugar cane and sugar beets; Fruit crops: bananas, oranges, apples, pears, breadfruit, pineapples, and cherries; Vegetable crops and tubers: tomatoes, lettuce, carrots, melons, asparagus, etc.; Nuts: cashews, peanuts, walnuts, pistachio nuts, almonds; Forage and turf grasses; Forage legumes: alfalfa, clover; Drug crops: coffee, cocoa, kola nut, poppy, tobacco; Spice and flavoring crops: vanilla, sage, thyme, anise, saffron, menthol, peppermint, spearmint, coriander. The terms “biofuels crops”, “energy crops”, “oil crops”, “oilseed crops”, and the like, to which the present methods and compositions can also be applied include the oil crops and further include plants such as sugarcane, castor bean, Camelina, switchgrass, Miscanthus, and Jatropha, which are used, or are being investigated and/or developed, as sources of biofuels due to their significant oil production and accumulation.

The terms “peptide”, “polypeptide”, and “protein” are used to refer to polymers of amino acid residues. These terms are specifically intended to cover naturally occurring biomolecules, as well as those that are recombinantly or synthetically produced.

The term “promoter” or “regulatory element” refers to a region or nucleic acid sequence located upstream or downstream from the start of transcription and which is involved in recognition and binding of RNA polymerase and/or other proteins to initiate transcription of RNA. Promoters need not be of plant or algal origin, for example, promoters derived from plant viruses, such as the CaMV35S promoter, or from other organisms, can be used in variations of the embodiments discussed herein. Promoters useful in the present methods include constitutive, tissue-specific, cell-type specific, seed-specific, inducible, repressible, and developmentally regulated promoters.

A skilled person appreciates that a promoter sequence can be modified to provide for a range of expression levels of an operably linked heterologous nucleic acid molecule. Less than the entire promoter region can be utilized and the ability to drive expression retained. However, it is recognized that expression levels of mRNA can be decreased with deletions of portions of the promoter sequence. Thus, the promoter can be modified to be a weak or strong promoter. A promoter is classified as strong or weak according to its affinity for RNA polymerase (and/or sigma factor); this is related to how closely the promoter sequence resembles the ideal consensus sequence for the polymerase. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts. The promoter of choice is preferably excised from its source by restriction enzymes, but can alternatively be PCR-amplified using primers that carry appropriate terminal restriction sites. It should be understood that the foregoing groups of promoters are non-limiting, and that one skilled in the art could employ other promoters that are not explicitly cited herein.

The term “purified” refers to material such as a nucleic acid, a protein, or a small molecule, such as galanthamine and/or hemanthamine and/or lycorine, which is substantially or essentially free from components which normally accompany or interact with the material as found in its naturally occurring environment, and/or which may optionally comprise material not found within the purified material's natural environment. The latter may occur when the material of interest is expressed or synthesized in a non-native environment. Nucleic acids and proteins that have been isolated include nucleic acids and proteins purified by standard purification methods. The term also embraces nucleic acids and proteins prepared by recombinant expression in a host cell as well as chemically synthesized nucleic acids. The present disclosure also encompasses methods and compositions comprising galanthamine and/or hemanthamine and/or lycorine. In some embodiments, the galanthamine and/or hemanthamine and/or lycorine is purified for therapeutic use and is formulated as a pharmaceutical composition. Such pharmaceutical compositions can be prepared by methods well known in the art. See, e.g., Remington: The Science and Practice of Pharmacy, 21^{st} Edition (2005), Lippincott Williams & Wilkins, Philadelphia, Pa.

“Recombinant” refers to a nucleotide sequence, peptide, polypeptide, or protein, expression of which is engineered or manipulated using standard recombinant methodology. This term applies to both the methods and the resulting products. As used herein, a “recombinant construct”, “expression construct”, “chimeric construct”, “construct” and “recombinant expression cassette” are used interchangeably herein.

As used herein, the phrase “sequence identity” or “sequence similarity” is the similarity between two (or more) nucleic acid sequences, or two (or more) amino acid sequences. Sequence identity is frequently measured as the percent of identical nucleotide or amino acid residues at corresponding positions in two or more sequences when the sequences are aligned to maximize sequence matching, i.e., taking into account gaps and insertions.

One of ordinary skill in the art will appreciate that sequence identity ranges are provided for guidance only. It is entirely possible that nucleic acid sequences that do not show a high degree of sequence identity can nevertheless encode amino acid sequences having similar functional activity. It is understood that changes in nucleic acid sequence can be made using the degeneracy of the genetic code to produce multiple nucleic acid molecules that all encode substantially the same protein. Means for making this adjustment are well-known to those of skill in the art. When percentage of sequence identity is used in reference to amino acid sequences it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Sequence identity (or similarity) can be readily calculated by known methods, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc., San Diego, Calif., United States of America), or by visual inspection. See generally, (Altschul, S. F. et al., J. Mol. Biol. 215: 403-410 (1990) and Altschul et al. Nucl. Acids Res. 25: 3389-3402 (1997)).

One example of an algorithm that is suitable for determining percent sequence identity and sequence similarity is the BLAST algorithm, which is described in (Altschul, S., et al., NCBI NLM NIH Bethesda, Md. 20894; & Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5877 (1993)). One measure of similarity provided by the BLAST algorithm is the smallest sum probability (P (N)), which provides an indication of the probability by which a match between two nucleotide or amino acid sequences would occur by chance. BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17: 149-163 (1993)) and XNU (Claverie and States, Comput. Chem., 17: 191-201 (1993)) low-complexity filters can be employed alone or in combination.

The constructs and methods disclosed herein encompass nucleic acid and protein sequences having sequence identity/sequence similarity at least about 75%, 80%, 85%, 90%, 95%, 96%, 97%, 98%, 99%, 100% to those specifically disclosed.

A “transgenic” organism, such as a transgenic plant, is a host organism that has been stably or transiently genetically engineered to contain one or more heterologous nucleic acid fragments, including nucleotide coding sequences, expression cassettes, vectors, etc. Introduction of heterologous nucleic acids into a host cell to create a transgenic cell is not limited to any particular mode of delivery, and includes, for example, microinjection, adsorption, electroporation, particle gun bombardment, whiskers-mediated transformation, liposome-mediated delivery, Agrobacterium-mediated transfer, the use of viral and retroviral vectors, etc., as is well known to those skilled in the art.

Conventional techniques of molecular biology, recombinant DNA technology, microbiology, chemistry useful in practicing the methods of the present disclosure are described, for example, in Green and Sambrook (2012) Molecular Cloning: A Laboratory Manual, Fourth Edition, Cold Spring Harbor Laboratory Press; Ausubel et al. (2003 and periodic supplements) Current Protocols in Molecular Biology, John Wiley & Sons, New York, N.Y.; Amberg et al. (2005) Methods in Yeast Genetics: A Cold Spring Harbor Laboratory Course Manual, 2005 Edition, Cold Spring Harbor Laboratory Press; Roe et al. (1996) DNA Isolation and Sequencing: Essential Techniques, John Wiley & Sons; J. M. Polak and James O'D. McGee (1990) In Situ Hybridization: Principles and Practice; Oxford University Press; M. J. Gait (Editor) (1984) Oligonucleotide Synthesis: A Practical Approach, IRL Press; D. M. J. Lilley and J. E. Dahlberg (1992) Methods in Enzymology: DNA Structure Part A: Synthesis and Physical Analysis of DNA, Academic Press; and Lab Ref: A Handbook of Recipes, Reagents, and Other Reference Tools for Use at the Bench, Edited by Jane Roskams and Linda Rodgers (2002) Cold Spring Harbor Laboratory Press; Burgess and Deutscher (2009) Guide to Protein Purification, Second Edition (Methods in Enzymology, Vol. 463), Academic Press. Note also U.S. Pat. Nos. 8,178,339; 8,119,365; 8,043,842; 8,039,243; 7,303,906; 6,989,265; US20120219994A1; and EP1483367B1. The entire contents of each of these texts and patent documents are herein incorporated by reference.

As used herein, “Pfs” refers to “hexahistidine-tagged methylthioadenosine/S-adenosylhomocysteine nucleosidase”.

### II. Overview of Several Embodiments

In an embodiment, the invention relates to a transgenic plant, comprising within its genome, and expressing, a heterologous nucleotide sequence coding for a class I O-methyltransferase. In yet another embodiment, the class I O-methyltransferase is a 4′-O-methyltransferase. In another embodiment, the 4′-O-methyltransferase is a norbelladine 4′-O-methyltransferase. In a further embodiment, the norbelladine 4′-O-methyltransferase converts norbelladine to 4′-O-methylnorbelladine. In one embodiment, the norbelladine 4′-O-methyltransferase is selected from among NpN4OMT1 (SEQ ID NO: 15), NpN4OMT2 (SEQ ID NO: 17), NpN4OMT3 (SEQ ID NO: 19), NpN4OMT4 (SEQ ID NO:21), and NpN4OMT5 (SEQ ID NO:23).

In a further embodiment, the invention contemplates a transgenic plant which further comprises: a heterologous nucleotide sequence encoding an enzyme that condenses 3,4-dihydroxybenzaldehyde and tyramine to form norbelladine, wherein said nucleotide sequence is expressed; and/or a heterologous nucleotide sequence encoding an enzyme that converts 4′-O-methylnorbelladine to N-demethylnarwedine, wherein said nucleotide sequence is expressed; and/or a heterologous nucleotide sequence encoding an enzyme that converts N-demethylnarwedine to N-demethylgalanthamine, wherein said nucleotide sequence is expressed; and/or a heterologous nucleotide sequence encoding an enzyme that converts N-demethylgalanthamine to galanthamine, wherein said nucleotide sequence is expressed; and/or a heterologous nucleotide sequence encoding an enzyme or enzymes that convert(s) 4′-O-methylnorbelladine to Noroxomaritidine, wherein said nucleotide sequence is expressed; and/or a heterologous nucleotide sequence encoding an enzyme or enzymes that convert(s) Noroxomaritidine to hemanthamine, wherein said nucleotide sequence is expressed; and/or a heterologous nucleotide sequence encoding an enzyme or enzymes that convert(s) 4′-O-methylnorbelladine to lycorine, wherein said nucleotide sequence is expressed. In another embodiment, the transgenic plant is selected from among a species of Galanthus, species of Brachypodium, species of Setaria, species of Populus, tobacco, corn, rice, soybean, cassava, canola (rapeseed), wheat, peanut, palm, coconut, safflower, sesame, cottonseed, sunflower, flax, olive, safflower, sugarcane, castor bean, switchgrass, Miscanthus, Camelina and Jatropha. In another embodiment, the heterologous nucleotide sequence is codon-optimized for expression in said transgenic plant. In another embodiment, the heterologous nucleotide sequence is expressed in a tissue or organ selected from among an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an axillary bud, a leaf, a stem, a root, a tuberous root, a rhizome, a tuber, a stolon, a corm, a bulb, an offset, a cell of said plant in culture, a tissue of said plant in culture, an organ of said plant in culture, and a callus.

The invention further contemplates a method of making a transgenic plant that produces galanthamine and/or hemanthamine and/or lycorine, comprising the steps of: a) inserting into the genome of a plant cell a heterologous nucleotide sequence comprising, operably linked for expression: (i) a promoter sequence; (ii) a nucleotide sequence encoding a protein selected from among: an O-methyltransferase selected from among a class I O methyltransferase, a 4′-O-methyltransferase, and a norbelladine 4′-0 methyltransferase; and/or a heterologous nucleotide sequence encoding an enzyme that condenses 3,4-dihydroxybenzaldehyde and tyramine to form norbelladine, wherein said nucleotide sequence is expressed; and/or a heterologous nucleotide sequence encoding an enzyme that converts 4′-O-methylnorbelladine to N-demethylnarwedine, wherein said nucleotide sequence is expressed; and/or a heterologous nucleotide sequence encoding an enzyme that converts N-demethylnarwedine to N-demethylgalanthamine, wherein said nucleotide sequence is expressed; and/or a heterologous nucleotide sequence encoding an enzyme that converts N-demethylgalanthamine to galanthamine, wherein said nucleotide sequence is expressed; and/or a heterologous nucleotide sequence encoding an enzyme or enzymes that convert(s) 4′-O-methylnorbelladine to Noroxomaritidine, wherein said nucleotide sequence is expressed; and/or a heterologous nucleotide sequence encoding an enzyme or enzymes that convert(s) Noroxomaritidine to hemanthamine, wherein said nucleotide sequence is expressed; and/or a heterologous nucleotide sequence encoding an enzyme or enzymes that convert(s) 4′-O-methylnorbelladine to lycorine, wherein said nucleotide sequence is expressed, b) obtaining a transformed plant cell; and c) regenerating from said transformed plant cell a genetically transformed plant, cells of which express said protein, wherein said genetically transformed plant produces galanthamine and/or hemanthamine and/or lycorine. In another embodiment, the protein-encoding nucleotide sequence is codon-optimized for expression in said transgenic plant. In another embodiment the protein-encoding nucleotide sequence is expressed in a tissue or organ selected from among an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an axillary bud, a leaf, a stem, a root, a tuberous root, a rhizome, a tuber, a stolon, a corm, a bulb, an offset, a cell of said plant in culture, a tissue of said plant in culture, an organ of said plant in culture, and a callus. In a still further embodiment, the invention relates to a transgenic plant made by a method as described above.

In an embodiment, the invention relates to a method of producing galanthamine and/or hemanthamine and/or lycorine in a plant, comprising expressing in cells of said plant a nucleotide sequence encoding an enzyme selected from among: an O-methyltransferase selected from among a class I O methyltransferase, a 4′-O-methyltransferase, and a norbelladine 4′-0 methyltransferase; and/or a heterologous nucleotide sequence encoding an enzyme that condenses 3,4-dihydroxybenzaldehyde and tyramine to form norbelladine, wherein said nucleotide sequence is expressed; and/or a heterologous nucleotide sequence encoding an enzyme that converts 4′-O-methylnorbelladine to N-demethylnarwedine, wherein said nucleotide sequence is expressed; and/or a heterologous nucleotide sequence encoding an enzyme that converts N-demethylnarwedine to N-demethylgalanthamine, wherein said nucleotide sequence is expressed; and/or a heterologous nucleotide sequence encoding an enzyme that converts N-demethylgalanthamine to galanthamine, wherein said nucleotide sequence is expressed; and/or a heterologous nucleotide sequence encoding an enzyme or enzymes that convert(s) 4′-O-methylnorbelladine to Noroxomaritidine, wherein said nucleotide sequence is expressed; and/or a heterologous nucleotide sequence encoding an enzyme or enzymes that convert(s) Noroxomaritidine to hemanthamine, wherein said nucleotide sequence is expressed; and/or a heterologous nucleotide sequence encoding an enzyme or enzymes that convert(s) 4′-O-methylnorbelladine to lycorine, wherein said nucleotide sequence is expressed, and, cultivating said plant for a time and under conditions wherein said plant produces galanthamine and/or hemanthamine and/or lycorine. In an embodiment, the nucleotide sequence is codon-optimized for expression in said transgenic plant. In a further embodiment, the nucleotide sequence is expressed in a tissue or organ from among an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an axillary bud, a leaf, a stem, a root, a tuberous root, a rhizome, a tuber, a stolon, a corm, a bulb, an offset, a cell of said plant in culture, a tissue of said plant in culture, an organ of said plant in culture, and a callus. In another embodiment, the method further comprising recovering galanthamine and/or hemanthamine and/or lycorine from said plant. And in another embodiment, the method further comprising purifying said galanthamine and/or hemanthamine and/or lycorine to a desired degree of purity. In another embodiment, the invention contemplates galanthamine and/or hemanthamine and/or lycorine produced by a method described above.

In yet another embodiment, the invention relates to a method of preparing a galanthamine and/or hemanthamine and/or lycorine-containing pharmaceutical composition, comprising formulating galanthamine and/or hemanthamine and/or lycorine as a pharmaceutical composition comprising a pharmaceutical carrier, dilient, or excipient, wherein said galanthamine is recovered from a transgenic plant. The invention further contemplates a pharmaceutical composition, wherein said transgenic plant is made by a method above described. In another embodiment, the invention relates to a pharmaceutical composition comprising galanthamine and/or hemanthamine and/or lycorine, wherein said galanthamine and/or hemanthamine and/or lycorine is obtained by growing a plant and recovering galanthamine and/or hemanthamine and/or lycorine from said plant.

The invention also relates to a method of treating Alzheimer's disease in a human patient in need thereof, comprising administering to said patient an effective amount of galanthamine, wherein said galanthamine is recovered from a transgenic plant; and/or wherein said transgenic plant is made by a method described above; and/or wherein said galanthamine is produced by a method described above. The invention further contemplates galanthamine for use in human therapy, wherein said galanthamine is recovered from a transgenic plant; and/or wherein said transgenic plant is made by a method described above; and/or wherein said galanthamine is produced by a method described above. In an embodiment, galanthamine is for use in treating Alzheimer's disease, wherein said galanthamine is recovered from a transgenic plant; and/or wherein said transgenic plant is made by a method described above; and/or wherein said galanthamine is produced by a method described above. In another embodiment, the invention relates to the use of galanthamine in human therapy, wherein said galanthamine is recovered from a transgenic plant; and/or wherein said transgenic plant is made by a method described above; and/or wherein said galanthamine is produced by a method described above.

In another embodiment, the invention relates to use of galanthamine for treating Alzheimer's disease, wherein said galanthamine is recovered from a transgenic plant of any; and/or wherein said transgenic plant is made by a method described above; and/or wherein said galanthamine is produced by a method described above. In another embodiment, the invention relates to use of galanthamine for the preparation of a medicament to treat Alzheimer's disease, wherein said galanthamine is recovered from a transgenic plant; and/or wherein said transgenic plant is made by a method described above; and/or wherein said galanthamine is produced by a method described above.

In another embodiment, the invention relates to a method of identifying genes in a biosynthetic pathway of an end product in an organism, comprising the steps of: a) confirming the presence of said end product in a tissue or tissues of said organism; b) identifying a gene or genes that co-expresses with accumulation of said end product; c) identifying and characterizing previously characterized homologs or orthologues, or naturally occurring variants of said gene or genes of step b; d) optionally, characterizing sequence motifs for one or more enzymes of step b or c; e) expressing nucleotide sequences encoding one or more enzymes of step b or c, and isolating and characterizing said enzyme or enzymes; f) optionally, performing phylogenetic analysis of said gene or genes identified in step c; g) optionally, determining the expression profile of said gene or genes identified in step c.

### III. Gene Discovery and Pathway Elucidation

There are several recent methodological improvements that can be used to expedite the gene discovery process. One is the sequencing revolution. With techniques such as illumina sequencing, transcriptomes can be assembled de novo from species for which the genome sequence is unknown. If this sequencing data comes from multiple tissues and/or time points, it can be used to determine relative expression levels for transcripts. In cases when one sequencing run yields more than sufficient data for one sample, multiple bar-coded samples can be run at the same time through multiplexing. Running multiple samples on the same lane removes lane to lane variation and reduces cost for sequencing. With one illumina sequencing experiment, both sequence information and expression profiles can be obtained for transcripts.

A second improvement is the increased number of characterized genes. With more identified genes than ever before, the probability that a gene being investigated is an orthologue of a previously studied gene is much higher. For example, with an E-value cut off of e⁻⁵, 58% of the ORFs in the Carthamus tinctorius transcriptome received an annotation. This knowledge of orthologues has particularly good coverage in plant O-methyltransferases (OMTs) that fall conveniently into two well defined classes when a phylogeny is constructed.

Lastly, there have been improvements in bioinformatic tools designed to handle large data sets. Some programs use statistics such as the Pearson correlation to find clusters of genes that co-express. Based on the genes in a particular group, a researcher can infer potential roles for unknown genes or new roles for previously characterized genes. An example is the discovery of a flavonol arabinosyltransferase from Arabidopsis. A cluster of co-expressing genes from flavonoid biosynthesis was used to identify additional genes within the cluster. Mutants of a gene in the flavonoid biosynthesis cluster with homology to arabinosyltransferases were tested for phenotypes. The resulting change in flavonoid profiles in these mutants were as expected for a flavonol arabinosyltransferase. Another approach is to use statistics including the Pearson coefficient to identify genes that correlate with a predefined model for gene behavior based on a hypothesis for how a set of genes of interest should express. This is of particular use when no genes involved in a pathway are known and therefore a cluster of interest cannot be readily identified. An example of a program designed to use the Pearson correlation in this way is HAYSTACK which has been used to identify genes regulated by the circadian clock.

A starting hypothesis when using this approach to construct models for completely unknown pathways is that biosynthetic genes will co-express in a pattern that matches the product accumulation pattern. In metabolism, biosynthetic gene expression tends to be correlated with the accumulation of end products, as in the case of anthocyanin and berberine biosynthesis. However, exceptions exist, such as the transport of nicotine from the site of biosynthesis in root to aerial parts of the plant. Thus, for nicotine accumulation, attempting to identify biosynthesis genes through co-expression/accumulation analysis in leaves could be misleading and/or uninformative. Therefore, although identification of candidate biosynthetic genes may begin with an in silico analysis of co-expression/accumulation patterns, an in vivo and/or in vitro type analysis is required to demonstrate that such candidate genes are important and/or involved in the accumulation of the end product or products.

### IV. Galanthamine Biosynthesis

Galanthamine is an Amaryllidaceae alkaloid used to treat the symptoms of Alzheimer's disease. This compound is primarily isolated from daffodil (Narcissus spp.), snowdrop (Galanthus spp.), and summer snowflake (Leucojum aestivum). Despite its importance as a medicine, no genes involved in the biosynthetic pathway of galanthamine have been identified. This absence of genetic information on biosynthetic pathways is a limiting factor in the development of synthetic biology platforms for many important botanical medicines. The paucity of information is largely due to the limitations of traditional methods for finding biochemical pathway enzymes and genes in non-model organisms. A new bioinformatic approach using several recent technological improvements was applied to search for genes in the proposed galanthamine biosynthetic pathway, first targeting methyltransferases due to strong signature amino acid sequences in the proteins. Using Illumina sequencing, a de novo transcriptome assembly was constructed for daffodil. BLAST was used to identify sequences that contain signatures for plant O-methyltransferases in this transcriptome. The program HAYSTACK was then used to identify methyltransferases that fit a model for galanthamine biosynthesis in leaf, bulb, and inflorescence tissues. One candidate gene for the methylation of norbelladine to 4′-O-methylnorbelladine in the proposed galanthamine biosynthetic pathway was identified. This methyltransferase cDNA was expressed in E. coli and the protein purified by affinity chromatography. The resulting protein was found to be a norbelladine 4′-O-methyltransferase (NpN4OMT) of the proposed galanthamine biosynthetic pathway. This work was further developed by using the expression profile of the N4OMT to find a cytochrome P450 capable for forming the compounds N-demethylnarwedine, (10aS,4bS)-noroxomaritidine and (10aR,4bR)-noroxomaritidine and a norbelladine synthase/reductase capable of forming norbelladine form 3,4-dihydroxybenzaldehyde and tyramine.

### V. Examples

The following examples are provided to illustrate various aspects of the present disclosure, and should not be construed as limiting the disclosure only to these particularly disclosed embodiments.

The materials and methods employed in the examples below are for illustrative purposes only, and are not intended to limit the practice of the present embodiments thereto. Any materials and methods similar or equivalent to those described herein as would be apparent to one of ordinary skill in the art can be used in the practice or testing of the present embodiments.

### Example 1: Identification of Galanthamine Biosynthetic Pathway Genes

This example describes the identification of biosynthetic pathway genes, specifically the identification of an enzyme within the Amaryllidaceae alkaloid biosynthetic pathway. This example further demonstrates the identification and selection of optimal candidates for transgenic gene expression by identifying closely related enzymes with optimal expression patterns, substrate specificity, cofactor requirements, low Kₘ for substrates, and kinetics and product formation.

### Plant Tissue and Chemicals

Daffodil plants were collected from an outdoor plot in St. Louis, Mo. during peak flowering and separated into leaf, bulb and inflorescence tissues. Inflorescence is considered all tissues above the spathe.

Formic acid, potassium phosphate monobasic, potassium phosphate dibasic, tris(hydroxymethyl)aminomethane, glycerol, sodium acetate, sodium chloride, tetramethylethylenediamine, calcium chloride, magnesium chloride and 3-mercaptoethanol were obtained from Acros Organics. Glycine, papaverine hydrochloride, S-adenosyl methionine (AdoMet), cobalt chloride, zinc chloride and manganese chloride were obtained from Fisher Scientific. Other chemicals include acetonitrile, JT Baker; InstaPAGE, IBI scientific; ethanol 200 proof, KOPTEC; Bradford reagent, Bio-Rad; S-adenosyl-L-homocysteine, Sigma-Aldrich; deoxynucleotide triphosphates (dNTPs), New England Biolabs (NEB); and isopropyl β-D-1-thiogalactopyranoside (IPTG), Gold Biotechnology. The norbelladine N-methylnorbelladine, 4′-O-methyl-N-methylnorbelladine and 4′-O-methylnorbelladine were synthesized previously. NotI, NdeI, T4 DNA ligase, Taq DNA polymerase and phusion High-Fidelity DNA polymerase enzymes were from New England Biolabs. M-MLV reverse transcriptase and RNaseOUT were obtained from Invitrogen.

### Alkaloid Extraction and Quantification

Daffodil leaf, bulb and inflorescence tissues were extracted by grinding tissue with a mortar and pestle cooled with liquid nitrogen. Each ground sample was split into three technical replicates. Two volumes of 70% ethanol were added followed by vortexing 5 min and centrifuging at 14,000×g for 10 min. The supernatant was filtered through a 0.2 m low protein binding hydrophilic LCR (PTFE, millex-LG) membrane. For galanthamine quantitation, samples were diluted 1000 fold. Liquid chromatography samples were injected (10 μl) onto an LC-20AD (Shimadzu) with a Waters Nova Pak C-18 (300×3.9 mm 4 m) column coupled to a 4000 QTRAP (AB Sciex Instruments) for MS/MS analysis. The gradient program had a flow rate of 0.8 ml/min; solvent A was 0.1% formic acid in H₂O and solvent B was 0.1% formic acid in acetonitrile. At the beginning of the program, solvent B was held at 15% for 2 min, followed by a linear gradient to 43% B at 15 min, 90% B at 15.1 min, 90% B at 20 min, 15% B at 21 min and 15% B at 26 min. A Turbo Ion Spray ionization source temperature of 500° C. was used with low resolution for Q1 and Q3. All multiple reaction monitoring (MRM) scans were performed in positive ion mode. The ion fragment used for quantitation of galanthamine was 288.00 [M+H]⁺/213.00 [M-OH—C₃H₇N]^{+•} m/z. Galanthamine was identified by comparison of retention time and fragmentation pattern to authentic galanthamine standard. The Analyst 1.5 software was used to quantitate galanthamine using a comparison of peak area of the unknown to authentic galanthamine.

### Illumina Sequencing and Transcriptome Assembly

The transcriptome was generated via data cleaning, short read assembly, final assembly, and post processing steps. A modified TRIzol RNA isolation method found as protocol number 13 in Johnson et al. was used to obtain RNA for cDNA library preparation. Illumina RNA-Seq was used to generate 100 base pair paired end reads from the cDNA library. The resulting data were monitored for overrepresented reads. Having found no such reads, we identified and removed adaptor sequences and sections of the phi X genome. The reads were then trimmed for quality using the FASTX toolkit with a Q value cut off of 10 as is default for PHRAP.

Reads were assembled in the following manner. ABySS was used to run multiple assemblies of the reads with a range of kmers 24≤k≤54. The resulting assemblies were assembled into scaffolds using ABySS scaffolder. Gaps in the sequences were resolved using GapCloser from the SOAPdenove suit. A final assembly was conducted on the resulting synthetic ESTs using Mira in EST assembly mode. All sequences with over 98% identity were considered redundant and removed using CD-Hit. The resulting contigs >100 base pairs long were included in the final assembly. Protein products for these contigs were predicted using ESTScan; all peptides over 30 amino acids were reported. Borrows-Wheeler Aligner was used to align the original reads to the assembled transcriptome to generate relative expression data for the contigs in leaf, bulb and inflorescence tissues. Anomalies in the number of reads per contig and abnormally long or short contigs were manually checked. To normalize for read depth, each expression value for each contig was divided by the total reads for the respective tissue and multiplied by 1 million. The Galanthus sp. and Galanthus elwesii transcriptomes were assembled in the same manner as for Narcissus sp. aff. pseudonarcissus. The Galanthus sp., Galanthus elwesii and Narcissus sp. aff. pseudonarcissus transcriptomes were also made using the Trinity pipeline. The same raw reads were assessed using FastQC followed by trimming with the FASTX tool kit. The fastx_trimmer was used to remove the first 13 bases and fastq_quality_trimmer was used to remove all bases on the 3′ end with a Phred quality score lower than 28. Sequences below 30 bases or without a corresponding paired end read were removed from the trimmed data set. Cleaned reads were input into the Trinity pipeline with default parameters for each data set. The unprocessed reads and trinity assemblies were used with the Trinity tool RNA-Seq by Expectation-Maximization (RSEM) to obtain the transcripts per million mapped reads (TPM) for all transcripts in each tissue (leaf, bulb and inflorescence) for each Trinity assembly. The Narcissus sp. aff. pseudonarcissus Trinity transcriptome was of inferior quality and was not used in further analysis.

### Candidate Gene Identification

Relative expression data were compared to the levels of galanthamine in daffodil tissues using HAYSTACK with a background cutoff of 1, correlation cutoff 0.8, fold cutoff 4 and p-value 0.05. Using BLASTP, a list of known methyltransferases was queried against the daffodil transcriptome peptide list with an E-value of e⁻⁹ to identify methyltransferase homologs. Accession numbers from NCBI for these methyltransferases are presented in Table 1.

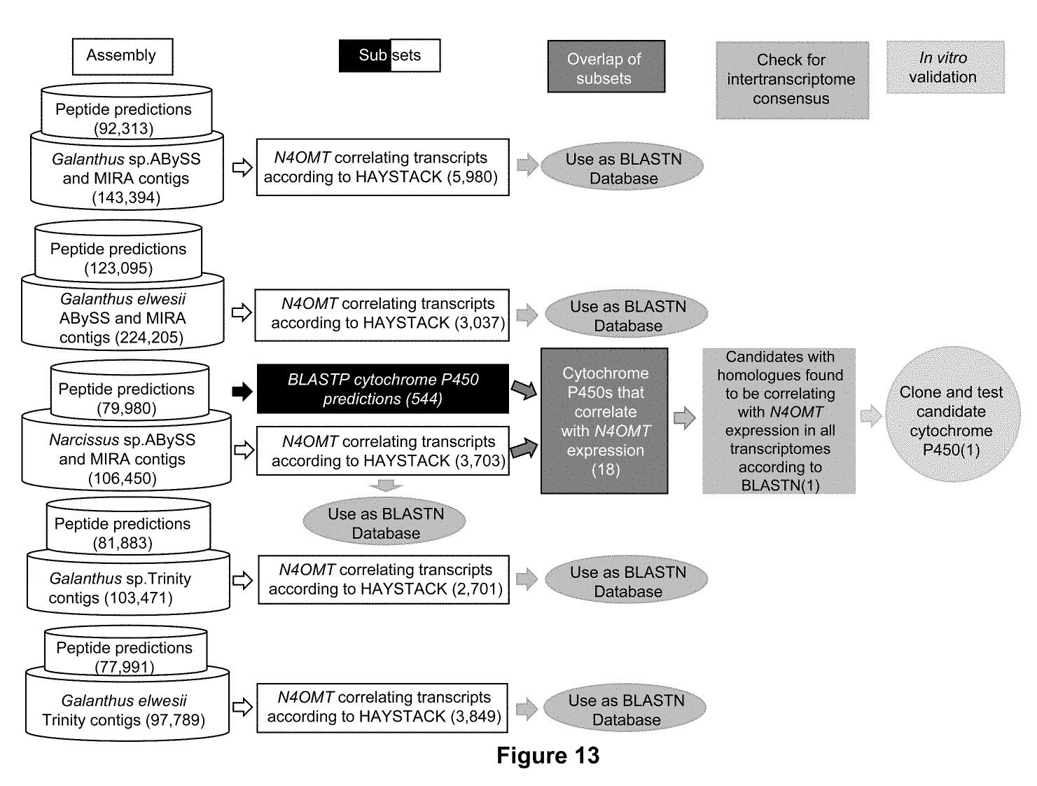

Overlap between the methyltransferase homologs and contigs that pass the HAYSTACK criteria were considered candidate genes. The candidate daffodil norbelladine 4′-OMT has the designation medp_9narc_20101112|62361 (SEQ ID NO:24 and SEQ ID NO:25). BLASTP with an e-value cut off of 1 e-4 was used to find homologs to known cytochrome P450 enzymes in all transcriptomes. A list of 472 unique, curated plant cytochrome P450 sequences from Dr. David Nelson, University of Tennessee, was used as a query against the ESTScan predicted peptides for each assembly. HAYSTACK was used to find correlations between the appropriate N4OMT expression model for each assembly (Table 2) and the transcripts in each assembly. All Galanthus models were based on the expression estimates for the closest NpN4OMT1 homologue in the assembly being used. The daffodil model was based on the RT-PCR data for NpN4OMT1 expression. HAYSTACK parameters are as follows: correlation cutoff <0.8, background cutoff >1, fold cutoff >4 and p-value cutoff <0.05. Homologues to annotated cytochrome P450 enzymes that were correlating with the N4OMT models were identified using BLASTN with an e-value cut off of 1 e-50 queried against the N4OMT co-expressing candidates in every other assembly. For each cytochrome P450 candidate, the total number of assemblies with a N4OMT co-expressing BLASTN hit were determined. Candidates present in 4-5 of the 5 comparable lists were considered top priority candidate genes and were cloned (FIG. 13). Among the top priority candidate genes were medp_9narc_20101112|22907 and medp_9narc_20101112|58880 for the cytochrome P450 and reductase searches respectively.

TABLE 1
Methyltransferases used in BLAST search

| Accession number | Substrate specificity | Reference |
|---|---|---|
| AAQ01669.1 | (R,S)-norcoclaurine, (R)-norprotosinomenine, | Ounaroon et al. (2003) |
| | (S)-norprotosinomenine, (R,S)-isoorientaline, | Plant J 36: 808-819 |
| AAQ01670.1 | | Unpublished |
| AAQ01668.1 | guaiacol, isovanillic acid, (R)-reticuline, | Ounaroon et al. (2003) |
| | (S)-reticuline, (R,S)-orientaline, (R)- | Plant J 36: 808-819 |
| | protosinomenine, (R,S)-laudanidine | |
| BAI79244.1 | (1S)-N-deacetylisoipecoside, (1R)-N- | Nomura and Kutchan |
| | deacetylipecoside, (13aR)-demethylalangi | (2010) J Biol Chem |
| | side, (11bS)-7′-O-demethylcephaeline, | 285: 7722-7738 |
| | (13aS)-redipecamine, (1R,S)-Isococlaurine, | |
| | (1R,S)-norcoclaurine, (1R,S)-isoorientaline, | |
| | oripavine | |
| BAI79245.1 | (13aS)-3-O-methylredipecamine, (1S)- | Nomura and Kutchan |
| | coclaurine, (1R,S)-N-methylcoclaurine, | (2010) J Biol Chem |
| | (1R,S)-4′-O-methylcoclaurine, (1R,S)-6-O- | 285: 7722-7738 |
| | methyllaudanosoline, (1R,S)-nororientaline, | |
| | (1S)-norreticuline, (1S)-reticuline, (13aS)- | |
| | coreximine | |
| BAI79243.1 | (1S)-N-deacetylisoipecoside, (1S)-7-O- | Nomura and Kutchan |
| | methyl-N-deacetylisoipecoside, (11bS)- | (2010) J Biol Chem |
| | cephaeline, (1R,S)-isococlaurine, (1R,S)- | 285: 7722-7738 |
| | norcoclaurine, (1S)-4′-O- | |
| | methyllaudanosoline, (1R,S)-nororientaline, | |
| | (1R,S)-isoorientaline, (1S)- | |
| | norprotosinomenine, (1R)- | |
| | norprotosinomenine, (1R,S)-protosinomenine | |
| BAA06192.1 | (R,S)-scoulerine | Takeshita et al. (1995) |
| | | Plant Cell Physiol 36: |
| | | 29-36 |
| AAD29843.1 | See reference | Takeshita et al. (1995) |
| | | Plant Cell Physiol 36: |
| | | 29-36 |
| AAD29841.1 | See reference | Takeshita et al. (1995) |
| | | Plant Cell Physiol 36: |
| | | 29-36 |
| AAD29845.1 | See reference | Takeshita et al. (1995) |
| | | Plant Cell Physiol 36: |
| | | 29-36 |
| AAD29842.1 | See reference | Takeshita et al. (1995) |
| | | Plant Cell Physiol 36: |
| | | 29-36 |
| AAD29844.1 | See reference | Takeshita et al. (1995) |
| | | Plant Cell Physiol 36: |
| | | 29-36 |
| BAC22084.1 | columbamine, | Morishige et al. (2002) |
| | tetrahydrocolumbamine, (S)-scoulerine, | Eur J Biochem 269: |
| | 2,3,9,10-tetrahydroxyprotoberberine | 5659-5667 |
| ACV50428.1 | Homology with Caffeoyl-CoA O- | Eswaran et al. (2010) |
| | methyltransferase described in Day et al. | BMC Biotechnol 10: |
| | (2009) Plant Physiol Biochem 47: 9-19. | 23 |
| AAN61072.1 | quercetin, 7-O-methylquercetin, | Ibdah et al. (2003) J |
| | quercetin-3-O-glucoside, quercetagetin, | Biol Chem 278: |
| | 3-O-methylquercetagetin 6-O- | 43961-43972 |
| | methylquercetagetin, 6-hydroxykaempferol, | |
| | myricetin, luteolin, caffeoyl-CoA | |
| AAR02420.1 | eriodictyol, homoeriodictyol, kaempferol, | Schroder et al. (2004) |
| | quercetin, isorhamnetin, chrysoeriol | Phytochemistry 65: |
| | | 1085-1094 |

TABLE 2
Models used in HAYSTACK analysis

| Model name | Leaf | Inflorescence | Bulb |
|---|---|---|---|
| ^{N} Daffodil N4OMT (relative units) | 1 | 30 | 45 |
| ^{N} Galanthus sp. N4OMT (RPM) | 0.01 | 33.34 | 139.79 |
| ^{N} Galanthus elwesii N4OMT (RPM) | 2.24 | 22.59 | 71.71 |
| ^{TC} Daffodil N4OMT (TPM) | NA | NA | NA |
| ^{T} Galanthus sp. N4OMT (TPM) | 2.42 | 29.02 | 94.73 |
| ^{T} Galanthus elwesii N4OMT (TPM) | 15.95 | 49.32 | 201.97 |

^{N} AbySS and MIRA assembly
^{C} homologue not found
^{T} Trinity assembly
RPM = reads per million
NA = not applicable

### Phylogenetic Tree

Sequences found in Table 3 were aligned using MUSCLE in the MEGA 5.2 software with default parameters.

TABLE 3
Methyltransferases used in phylogeny

| Accession | Short | | | |
|---|---|---|---|---|
| number | name | Species | Substrate specificity | Reference |
| AAQ01669.1 | PsN6OMT | Papaver | (R,S)-norcoclaurine, (R)- | Ounaroon |
| | | somniferum | norprotosinomenine, (S)- | et al. |
| | | | norprotosinomenine, (R,S)- | (2003) |
| | | | isoorientaline, | Plant J 36: |
| | | | | 808-819 |
| AAQ01670.1 | PsCOMT | Papaver | | Unpublished |
| | | somniferum | | |
| AAQ01668.1 | PsR7OMT | Papaver | guaiacol, isovanillic acid, (R)- | Ounaroon |
| | | somniferum | reticuline, (S)-reticuline, | et al. |
| | | | (R,S)-orientaline, (R)- | (2003) |
| | | | protosinomenine, (R,S)- | Plant J 36: |
| | | | laudanidine | 808-819 |
| 179244.1 | PiOMT2 | Psychotria | (1S)-N-deacetylisoipecoside, | Nomura |
| | | ipecacuanha | (1R)-N-deacetylipecoside, | and |
| | | | (13aR)-demethylalangiside, | Kutchan |
| | | | (11bS)-7′-O- | (2010) J |
| | | | demethyl cephaeline, (13aS)- | Biol Chem |
| | | | redipecamine, (1R,S)- | 285: 7722- |
| | | | isococlaurine, (1R,S)- | 7738 |
| | | | norcoclaurine, (1R,S)- | |
| | | | isoorientaline, oripavine | |
| BAI79243.1 | PiOMT1 | Psychotria | (1S)-N-deacetylisoipecoside, | Nomura |
| | | ipecacuanha | (1S)-7-O-methyl-N- | and |
| | | | deacetylisoipecoside, (11bS)- | Kutchan |
| | | | cephaeline, (1R,S)- | (2010) J |
| | | | Isococlaurine, (1R,S)- | Biol Chem |
| | | | norcoclaurine, (1S) 4′O- | 285: 7722- |
| | | | methyllaudanosoline, (1R,S)- | 7738 |
| | | | nororientaline, (1R,S)- | |
| | | | isoorientaline, (1S)- | |
| | | | norprotosinomenine, (1R)- | |
| | | | norprotosinomenine, (1R,S)- | |
| | | | protosinomenine | |
| BAA06192.1 | CjS9OMT | Coptis japonica | (R,S)-scoulerine | Takeshita |
| | | | | et al. |
| | | | | (1995) |
| | | | | Plant Cell |
| | | | | Physiol |
| | | | | 36: 29-36 |
| AAD29843.1 | TtCOMT3 | Thalictrum | See reference | Frick et al. |
| | | tuberosum | | (1999) |
| | | | | Plant J 17: |
| | | | | 329-339 |
| AAD29841.1 | TtCOMT1 | Thalictrum | See reference | Frick et al. |
| | | tuberosum | | (1999) |
| | | | | Plant J 17: |
| | | | | 329-339 |
| AAD29845.1 | TtCOMT5 | Thalictrum | See reference | Frick et al. |
| | | tuberosum | | (1999) |
| | | | | Plant J 17: |
| | | | | 329-339 |
| AAD29842.1 | TtCOMT2 | Thalictrum | See reference | Frick et al. |
| | | tuberosum | | (1999) |
| | | | | Plant J 17: |
| | | | | 329-339 |
| AAD29844.1 | TtCOMT4 | Thalictrum | See reference | Frick et al. |
| | | tuberosum | | (1999) |
| | | | | Plant J 17: |
| | | | | 329-339 |
| BAC22084.1 | CjCOMT | Coptis japonica | columbamine, | Morishige |
| | | | tetrahydrocolumbamine, (S)- | et al. |
| | | | scoulerine, 2,3,9,10- | (2002) Eur |
| | | | tetrahydroxyprotoberberine | J Biochem |
| | | | | 269: 5659- |
| | | | | 5667 |
| ACV50428.1 | JcCCoAOMT | Jatropha curcas | Homology with caffeoyl-CoA | Eswaran et |
| | | | O-methyltransferase | al. (2010) |
| | | | described in Day et al. (2009) | BMC |
| | | | Plant Physiol Biochem 47: 9- | Biotechnol |
| | | | 19 | 10: 23 |
| AAR02420.1 | CrF4OMT | Catharanthus | eriodictyol, homoeriodictyol, | Schroder |
| | | roseus | kaempferol, quercetin, | et al. |
| | | | isorhamnetin, chrysoeriol | (2004) |
| | | | | Phytoche |
| | | | | mistry 65: |
| | | | | 1085-1094 |
| Q9C5D7.1 | AtCCoAOMT | Arabidopsis | N.D. | Ibrahim et |
| | | thaliana | | al. (1998) |
| | | | | Plant Mol |
| | | | | Biol 36: 1- |
| | | | | 10 |
| C7AE94.1 | VvAOMT | Vitis vinifera | cyanidin 3-glucoside, | Hugueney |
| | | | delphinidin 3-glucoside, | et al. |
| | | | quercetin 3-glucoside, | (2009) |
| | | | cyanidin, quercetin, | Plant |
| | | | myricetin, pelargonidin 3- | Physiol |
| | | | glucoside, catechin, | 150: 2057- |
| | | | epicatechin | 2070 |
| ADZ76153.1 | VpOMT4 | Vanilla planifolia | tricetin, 5-hydroxyferulic acid | Widiez et |
| | | | ethyl ester, 5-hydroxyferulic | al. (2011) |
| | | | acid, myricetin, 3,4- | Plant Mol |
| | | | dihydroxybenzaldehyde, | Biol 76: |
| | | | Quercetin, 5- | 475-488 |
| | | | hydroxyconiferaldehyde, | |
| | | | caffeoyl CoA, caffeic acid | |
| | | | ethyl ester, caffeoylaldehyde, | |
| | | | caffeic acid | |
| ADZ76154.1 | VpOMT5 | Vanilla planifolia | tricetin, 5-hydroxyferulic acid | Widiez et |
| | | | ethyl ester, 5-hydroxyferulic | al. (2011) |
| | | | acid, myricetin, 3,4- | Plant Mol |
| | | | dihydroxybenzaldehyde, | Biol 76: |
| | | | quercetin, 5- | 475-488 |
| | | | hydroxyconiferaldehyde, | |
| | | | caffeoyl CoA, caffeic acid | |
| | | | ethyl ester, caffeoylaldehyde, | |
| | | | caffeic acid | |
| Q84KK6 | GeI4OMT | Glycyrrhiza | 2,7,4′-trihydroxyisoflavanone, | Akashi et |
| | | echinata | medicarpin | al. (2003) |
| | | | | Plant Cell |
| | | | | Physiol |
| | | | | 44: 103- |
| | | | | 112 |
| C6TAY1 | GmF4OMT | Glycine max | apigenin, daidzein, genistein, | Kim et al. |
| | | | quercetin, naringenin | (2005) J |
| | | | | Biotechnol |
| | | | | 119: 155- |
| | | | | 162 |
| AAY89237.1 | LuCCoA3OMT | Linum | | Nestor et |
| | | usitatissimum | | al. (2008) |
| 3C3Y\|A | McPFOMT | Mesembryanthemum | quercetin, quercetagetin, | Kopycki et |
| | | crystallinum | caffeic acid, CoA, caffeoyl | al. (2008) |
| | | | glucose | J Mol Biol |
| | | | | 378: 154- |
| | | | | 164 |
| 62361_DF6 | NpN4OMT1 | Narcissus | norbelladine, N- | This study |
| | | pseudonarcissus | methylnorbelladine, | |
| | | cv. ‘Carlton’ | dopamine | |
| BAB71802.1 | OCNMT | Coptis japonica | (R)-coclaurine, (S)-coclaurine, | Choi et al. |
| | | | (R,S)-norreticuline, (R,S)- | (2002) J |
| | | | norlaudanosoline, (R,S)-6-O- | Biol Chem |
| | | | methylnorlaudanosoline, 6,7- | 277: 830- |
| | | | dimethoxyl-1,2,3,4- | 835 |
| | | | tetrahydroisoquinoline, 1- | |
| | | | methyl-6,7-dihydroxy- | |
| | | | 1,2,3,4- | |
| | | | tetrahydroisoquinolinne | |
| BAB12278.1 | CsCNMT | Camellia sinensis | 7-methylxanthine, 3- | Kato et al. |
| | | | methylxanthine, 1- | (2000) |
| | | | methylxanthine, theobromine, | Nature |
| | | | theophylline, paraxanthine | 406: 956- |
| | | | | 957 |
| Q93WU3 | ObCV4OMT | Ocimum | chavicol, phenol, eugenol, t- | Gang et al. |
| | | basilicum | isoeugenol, t-anol | (2002) |
| | | | | Plant Cell |
| | | | | 14: 505- |
| | | | | 519 |
| Q8WZO4 | HsCOMT | Homo sapiens | A catechol | |
| 3CBG1A | SynOMT | Cyanobacterium | hydroxyferulic acid, caffeic | Kopycki et |
| | | Synechocystis | acid, caffeoyl-CoA, | al. (2008) |
| | | sp. Strain PCC 6803 | caffeoylglucose, 3,4,5- | J Biol |
| | | | trihydorxycinnamic acid, | Chem 283: |
| | | | tricetin, 3,4-dihydroxybenzoic | 20888- |
| | | | acid | 20896 |

For the phylogeny, this alignment was provided as input into the Maximum-Likelihood algorithm also found in MEGA 5.2. Default parameters were used except the Gaps/Missing Data treatment was set to partial deletion.

### PCR and Cloning

The 5′ and 3′ ends of the NpN4OMT sequence were completed using Rapid Amplification of cDNA Ends (RACE) with the Invitrogen RACE kit. SEQ ID NOs:1-13 in the sequence listing describe gene specific primers (GSP) used in RACE, cloning and colony PCR.

The same PCR program was used for both 5′ and 3′RACE. This applies to both cycles of nested PCR as well. The PCR program parameters were 30 seconds 98° C. 1 cycle; 10 seconds 98° C., 30 seconds 60° C., 1 min 72° C. 30 cycles; 5 min 72° C. 1 cycle. The outer-primer PCR was a mixture of 4.6 ng/μl RACE ready bulb cDNA, 0.3 mM dNTPs, 0.3 μM GSP primer, 0.9 μM kit provided RACE primer, 1 U NEB phusion High-fidelity DNA polymerase and Invitrogen recommended quantity of buffer in a 50 μl reaction. The inner-primer PCR used the product of the outer-primer PCR as template with 0.2 μM of the inner RACE GSP and Invitrogen primers and 0.2 mM dNTPs.

Amplification of the NpN4OMT open reading frame was performed with 5.1 ng/μl daffodil bulb oligo(dT) primed cDNA, 0.4 mM dNTPs, 0.4 μM each forward and reverse outer primer, 1 UNEB Phusion High-Fidelity DNA Polymerase and recommended buffer in a 50 μl reaction with the following PCR program parameters: 30 seconds 98° C. 1 cycle; 10 seconds 98° C., 30 seconds 52° C., 1 min 72° C. for 30 cycles; 5 min 72° C. 1 cycle. The inner-primer PCR used 1 μl of the outer-primer PCR product and used the inner primers in SEQ ID NO:1-13. The same PCR time program was used except the annealing temperature was increased to 53° C.

NpN4OMT was cloned into the pET28a vector with the NotI and NdeI restriction sites that were added to the 5′ and 3′ ends of the open reading frame using the inner PCR primers. PCR product and pET28a were digested with NotI and NdeI enzymes, followed by gel purification and ligation with the T4 DNA ligase. The resulting construct was transformed into E. coli DH5a cells and screened on Luria-Bertani agar plates with 50 μg/ml kanamycin. Resulting colonies were screened by colony PCR with T7 sequencing and T7 terminator primers and Taq DNA polymerase. The following cycle program was used: 3 min 94° C. 1 cycle; 30 s 94° C., 30 s 52° C., 2 min 72° C. 30 cycles; 7 min 72° C. 1 cycle. Plasmid minipreps were obtained using the QIAGEN QIAprep Spin Miniprep Kit. After Sanger sequencing of constructs (Genewiz), the desired plasmids were transformed into E. coli BL21(DE3) Codon Plus RIL competent cells. The sequences of the resulting 5 variants have the following accession numbers KJ584561(NpN4OMT1; SEQ ID NO:14), KJ584562(NpN4OMT2; SEQ ID NO: 16), KJ584563(NpN4OMT3; SEQ ID NO: 18), KJ584564(NpN4OMT4; SEQ ID NO:20) and KJ584565(NpN4OMT5; SEQ ID NO:22). Cloning of CYP96T1 into the pVL1392 vector and the norbelladine synthase/reductase into the pET28a vector was done using methods similar to those used in the cloning of NpN4OMT.

### Protein Purification

Recombinant protein production of NpN4OMT and norbelladine synthase/reductase in 1 L of E. coli and purification with TALON resin followed standard methods. No proteases were added to the protein extract, and desalting was performed with PD-10 columns from GE Healthcare. Protein quantity was determined according to Bradford; purity was monitored by SDS-PAGE. The E. coli cell line containing the hexahistidine-tagged methylthioadenosine/S-adenosylhomocysteine nucleosidase (Pfs) construct from Choi-Rhee and Cronan's work was used to purify Pfs protein. CYP96T1 was co-expressed with cytochrome P450 reductase in Spodoptera frugiperda Sf9 cells using Baculogold baculoviurus (BD Biosciences). Whole cell lysates were used in CYP96T1 enzyme assays.

### Screening Enzyme Assays

Enzyme assays for initial testing of NpN4OMT1 contained 10 μg of pure protein with 200 μM AdoMet, 100 μM norbelladine and 30 mM potassium phosphate buffer pH 8.0 in 100 μl. The assays were incubated for 2 hr at 30° C. The vector control was an E. coli extract purified with TALON in the same way as the methyltransferase protein. For the vector control assay, an equal volume of the pure vector control extract was substituted for the NpN40MT1 protein in the enzyme assay. These assays were quenched by adjusting the pH to 9.5 with two volumes of sodium bicarbonate and extracted with two volumes ethyl acetate two times. After drying, the extracts were re-suspended in the initial mobile phase of the HPLC program. The HPLC separation of the assays was performed using a phenomenex Luna C8(2) 5 m 250×4.6 mm column with solvent A (0.1% formic acid in H₂O) and solvent B (acetonitrile). The program started with 10% solvent B and a flow rate of 0.8 ml/min, a linear gradient began at 2 min to 30% at 15 min, 90% at 15.1 min, 90% at 20 min, 10% at 21 min and 10% at 28 min. Injection volume was 20 μl using a Waters auto-sampler. Waters UV detector was set to 277 nm.

CYP96T1 assays contained 30 mM KPO₄ pH 8.0, 1.25 mM NADPH, 10 μM substrate and 70 μl of virus infected Sf9 cell suspension in 200 μl total volume. The assays were incubated for 2-4 hr at 30° C. 4′-O-metylnorbelladine was used as an initial test compound. Substrate specificity tests were done on 4′-O-methyl-N-methylnorbelladine, norbelladine, N-methylnorbelladine, 3′-O-methylnorbelladine, 3′,4′-O-dimethylnorbelladine, haemanthamine, (S)-coclaurine, (R)-coclaurine and mixed (10aR,4bR)- and (10aS,4bS)-noroxomaritidine. Assays derivatized with sodium borohydride were incubated 2 hr at 30° C. followed by addition of 0.5 volumes 0.5 M sodium borohydride in 0.5 M sodium hydroxide and incubated 30 min at RT. The CYP96T1 assay resolved on a Chiral-CBH column and assays measured with HPLC used fresh CYP96T1 and CPR expressing SF9 cell protein prepared using re-amplified virus. Enzyme assays on all substrates were extracted as previously described and run on a QTRAP 4000 coupled to a IL-20AC XR prominence liquid auto sampler, 20AD XR prominence liquid chromatograph and Phenomenex Luna 5 μm C8(2) 250×4.60 mm column. HPLC gradient and MS settings were as previously described for NpN4OMT. Assay specific MS/MS parameters are presented in

Initial screening assays for norbelladine synthase contained 0.1 M sodium phosphate buffer pH 7.0, 1 mM NADPH, 1 mM tyramine, 1 mM 3,4-dihydroxybenzaldehyde and 10 μg pure protein. They were incubated at 30° C. for 2 hr. Assays were extracted with Ethyl acetate at pH 9.5 as in NpN4OMT and CYP96T1 assays. The extracts were re-suspended in mobile phase matching the composition of the HPLC program. Samples were run with the same LC-MS/MS hardware set up and time program as in the CYP96T1 work. MS/MS parameters used to specifically monitor m/z 260 for norbelladine are collision energy 15, decluttering potential 50 and m/z 260.00.

Table 4. Multiple Reaction Monitoring (MRM) parameters for relative quantification of (10aR,4bR)- and (10aS,4bS)-noroxomaritidine, N-demethylnarwedine, narwedine and the two unknown compounds are presented in Table5. For analysis of product chirality, a Chrom Tech, Inc. Chiral-CBH 100×4.0 mm, 5 μM column was used with a 30 min isocratic flow of 2.5% HPLC grade ethanol and 10 mM ammonium acetate with pH adjusted to 7.0 with ammonium hydroxide.

Initial screening assays for norbelladine synthase contained 0.1 M sodium phosphate buffer pH 7.0, 1 mM NADPH, 1 mM tyramine, 1 mM 3,4-dihydroxybenzaldehyde and 10 μg pure protein. They were incubated at 30° C. for 2 hr. Assays were extracted with Ethyl acetate at pH 9.5 as in NpN4OMT and CYP96T1 assays. The extracts were re-suspended in mobile phase matching the composition of the HPLC program. Samples were run with the same LC-MS/MS hardware set up and time program as in the CYP96T1 work. MS/MS parameters used to specifically monitor m/z 260 for norbelladine are collision energy 15, decluttering potential 50 and m/z 260.00.

TABLE 4
MS/MS parameters for CYP96T1 substrate tests

| | Product specific | Substrate specific |
|---|---|---|
| | parameters | parameters |
| Substrate | (CE)(DP)(Q1 m/z) | (CE)(DP)(Q1 m/z) |
| 4′-O-Methylnorbelladine | (35)(70)(272.30) | (20)(60)(274.30) |
| 4′-O-Methyl-N- | (35)(70)(286.20) | (20)(60)(288.30) |
| methylnorbelladine | | |
| 3′-O-Methylnorbelladine | (35)(70)(272.30) | (35)(60)(274.30) |
| 3′,4′-O- | (35)(70)(286.20) | (20)(60)(288.30) |
| Dimethylnorbelladine | | |
| Norbelladine | (35)(60)(258.00) | (15)(50)(260.00) |
| N-Methylnorbelladine | (35)(70)(272.30) | (20)(60)(274.30) |
| Haemanthamine | (35)(70)(300.12)/ | (35)(70)(302.14) |
| | (35)(70)(318.13)^{HO} | |
| (10aR,4bR)- and | (35)(70)(270.30)/ | (35)(70)(272.30) |
| (10aS,4bS)- | (35)(70)(288.30)^{HO} | |
| Noroxomaritidine | | |
| Isovanillin and tyramine | (20)(40)(290.30)^{a}/ | (20)(60)(138.20)/ |
| | (20)(60)(272.20)^{b}/ | (20)(50)(153.20) |
| | (35)(70)(270.20)^{c} | |
| (S)-Coclaurine | (35)(70)(284.30)/ | (20)(70)(286.30) |
| | (30)(60)(570.60)^{dim} | |
| (R)-Coclaurine | (35)(70)(284.30)/ | (20)(70)(286.30) |
| | (30)(60)(570.60)^{dim} | |
| 4′-O-Methylnorbelladine | (20)(60)(274.30) | (20)(60)(274.30) |
| assays followed by sodium | | |
| borohydride | | |
| derivatization | | |

^{HO}hydroxylation monitored
^{dim}dimer formation monitored
^{a}C-C phenol coupling with no amine aldehyde condensation
^{b}amine aldehyde condensation/amine aldehyde condensation with C-C phenol coupling and a reduction.
^{c}amine aldehyde condensation with C-C phenol coupling

TABLE 5
MS/MS parameters used in MRM studies

| | MRM parameters(CE)(DP) |
|---|---|
| Compound(C-C phenol coupling type) | (Q1 m/z)(Q2 m/z)(RT min) |
| Noroxomaritidine(para′-para) | (35)(70)(272.3)(229.0)(5.3) |
| N-Demethylnarwedine(para′-ortho) | (35)(70)(272.3)(201.0)(7.9) |
| 4′-O-Methyl-N-methylnorbelladine assay | (35)(70)(286.1)(271.0)(4.7) |
| unknown 1(potential para′-para product) | |
| 4′-O-Methyl-N-methylnorbelladine assay | (30)(70)(286.1)(243.0)(7.5) |
| unknown 2 (potential ortho′-para product) | |
| Narwedine(para′-ortho) | (30)(70)(286.1)(229.1)(8.1) |

### Kinetic Characterization

After optimization of the NpN4OMT assay, the buffer was changed to 100 μM glycine at pH 8.8, with 5 mM of MgCl₂ added and the temperature was increased to 37° C. in 100 μl total reaction volume. When performing kinetic assays, the E. coli enzyme Pfs was added to break down SAH and prevent product inhibition. Papaverine was used as an internal standard.

With the same solvent system as for screening enzyme assays, the HPLC program started with 20% B and a flow rate of 0.8 ml/min, a linear gradient began at 2 min to 25.4% B at 7 min, 90% at 7.2 min, 90% at 9 min, 20% at 9.1 min and 20% at 14 min. A 4000 QTRAP mass spectrometer coupled to the same LC column and time program as used in HPLC was used to collect all compound mass and fragmentation data. Fragmentation data and program setting details are shown in Table 6.

TABLE 6
Parameters used for LC/MS/MS analysis

| | Predicted | | | | |
|---|---|---|---|---|---|
| | molecular | | CE | DP | Injection |
| | ion m/z | Fragments m/z (% relative | value | value | volume |
| Compound | [M + H] | intensity)[proposed fragment] | (V) | (V) | (μl) |
| galanthamine | 288.14 | | 35 | 70 | 10 |
| norbelladine* | 260.13 | 121.04(100.00)[M − OH— | 15 | 60 | 10 |
| | | C₈H₉O]⁺*, 121.84(19.62), | | | |
| | | 122.00(13.29)[M + H—C₇H₈O]⁺, | | | |
| | | 122.64(10.13), | | | |
| | | 123.04(38.61)[M − C₈H₁₀ON]⁺*, | | | |
| | | 123.68(11.39), 138.00(3.16)[M − | | | |
| | | C₈H₉O]⁺*, 260.16(21.52)[M + H]⁺ | | | |
| 4′-O- | 274.14 | 122.08(1.63)[M + H—C₈H₁₀O₂N]⁺, | 35 | 60 | 10 |
| methylnorbelladine* | | 137.04(100.00) [M − C₈H₁₀ON]⁺*, | | | |
| | | 274.08(2.45)[M + H]⁺ | | | |
| N- | 274.14 | 121.04(100.00)[M − C₈H₁₀O₂N]⁺*, | 20 | 60 | 10 |
| methylnorbelladine | | 121.52(19.11), 122.00(18.18), | | | |
| | | 123.04(82.29)[M − C₉H1₂ON]⁺* , | | | |
| | | 123.68(17.69), 124.00(16.43), | | | |
| | | 124.56(15.03), 124.96(10.53), | | | |
| | | 152.16(73.72)[M − C₈H₉O]⁺* , | | | |
| | | 274.08(28.54)[M + H]⁺, | | | |
| 4′-O-methyl-N- | 288.18 | 137.04(100.00)[M − C₉H₁₂ON]⁺*, | 20 | 60 | 10 |
| methylnorbelladine* | | 150.08(1.22)[M − C₈H₉O]⁺*, | | | |
| | | 288.08(18.67)[M + H]⁺ | | | |
| dopamine* | 154.09 | 91.04(41.26), 119.04(24.85)[M − | 20 | 70 | 20 |
| | | OH—OH]⁺*, | | | |
| | | 137.04(100.00)[M + H—OH]⁺, | | | |
| | | 137.92(10.21), | | | |
| | | 154.08(1 .29)[M + H]⁺ | | | |
| 3′-O- | 168.10 | 90.96(47.83), 91.60(10.87)[M − | 20 | 70 | 20 |
| methyldopamine | | OH—CH₃—C₂H₆N]⁺*, | | | |
| | | 94.88(11.87), 95.20(10.87), | | | |
| | | 118.72(15.22), | | | |
| | | 119.04(39.13)[M − OH—OCH₃]⁺*, | | | |
| | | 140.20(13.04)[M − CH—CH₃]⁺, | | | |
| | | 152.40(10.87)[M + H—NH2]⁺, | | | |
| | | 151 .04(100.00)[M + H—OH]⁺, | | | |
| | | 151.60(13.04), | | | |
| | | 168.16(52.17)[M + H]⁺, | | | |
| methylated | 168.10 | 91.04(41.18)[M − OH—CH₃— | 20 | 70 | 20 |
| dopamine product | | C₂H₆N]⁺*, 92.08(11.76)[M + H— | | | |
| | | OH—CH₃—C₂H₆N]⁺, | | | |
| | | 109.28(11.76)[M + H—CH₃— | | | |
| | | C₂H₆N]⁺, 112.08(17.65), | | | |
| | | 119.04(29.41)[M − OH—OCH₃]⁺*, | | | |
| | | 123.00(17.65)[M − C₂H₆N]⁺*, | | | |
| | | 126.00(11.76), | | | |
| | | 136.00(17.65)[M + H—OH—CH₃]⁺, | | | |
| | | 150.56(17.65)[M − OH]⁺*, | | | |
| | | 151.04(100.00)[M + H—OH]⁺, | | | |
| | | 151.60(17.65), 154.32(17.65), | | | |
| | | 168.08(94.12)[M + H]⁺, | | | |
| | | 168.48(17.65), 169.68(11.76), | | | |
| papaverine | 340.16 | 171.12(47.37)[M − C₈H₉O₂— | 52 | 70 | 10 |
| | | OCH₃]⁺*, 172.08(11.94)[M + H— | | | |
| | | C₈H₉O₂—OCH₃]⁺, | | | |
| | | 187.04(11.23)[M − C₈H₉O₂— | | | |
| | | CH₃]⁺*, 202.08(48.17)[M − | | | |
| | | C₈H₉O₂]⁺*, 280.08(17.59)[M + H− | | | |
| | | N—CH₃—OCH₃]⁺, | | | |
| | | 296.08(16.81)[M + H—N—CH₃— | | | |
| | | CH₃]⁺, 308.08(25.35)[M − | | | |
| | | OCH₃]+*, 324.08(100.00)[M − | | | |
| | | CH₃]⁺*, 340.08(1.16)[M + H]⁺ | | | |

*Cut off for inclusion in fragments is 10% relative intensity. If parent ions or fragments used in MRM are below this threshold, these ions are reported.

For NpN4OMT norbelladine kinetics an MRM program in positive ion mode was used to monitor the following fragments 260.00 [M+H]⁺/138.00 [M−C₈H₉O]^{+•} m/z, 260.00 [M+H]⁺/121.00 [M−C₇H₈NO₂]^{+•} m/z, 274.00 [M+H]⁺/137.00 [M+H—C₈H₉O₂]⁺ m/z, 274.00 [M+H]⁺/122.00 [M+H—C₈H₁₀NO₂]⁺ m/z. The fragments with 260.00 [M+H]⁺ m/z and 274.00 [M+H]⁺ m/z molecular ions were replaced when looking at N-methylnorbelladine for 274.00 [M+H]⁺/152.10 [M−C₈H₉O]^{+•} m/z, 274.00 [M+H]⁺/121.00 [M−C₉H₁₂NO₂]^{+•} m/z, 288.00 [M+H]⁺/150.10 [M−C₈H₉O₂]^{+•} m/z and 288.20 [M+H]⁺/137.00 [M−C₉H₁₂NO]^{+•} m/z. Papaverine internal standard was monitored with the following fragments 340.40[M+H]⁺/324.20 [M−CH₃]^{+•} m/z and 340.40 [M+H]⁺/202.10 [M−C₈H₉O₂]^{+•} m/z. When conducting dopamine kinetics, galanthamine was used as the internal standard and samples were not ethyl acetate extracted prior to LC/MS/MS analysis. To remove protein, two volumes of acetonitrile were added followed by 1 hr at −20° C. and 10 min centrifugation at 16,100×g, 4° C. The supernatant was dried under vacuum and re-suspended in the starting mobile phase before analysis. The HPLC time program was changed to start at 5% solvent B with solution going to waste until 3.9 min, at 5 min start linear gradient to 25% B at 25 min, 90% B at 9.5 min, 90% B at 11 min, 5% B at 11.1 min and 5% B at 16 min. Ions monitored in the MRM were 168.00 [M+H]⁺/151.00 [M+H-OH]⁺ m/z and 168.00 [M+H]⁺/119.00 [M−OH—OCH₃]^{+•} m/z. AdoMet steady state kinetic parameters were determined with norbelladine as the saturated substrate. Product was quantitated using HPLC with the 28 min program used for screening enzyme assays. Product for assays on the additional NpN4OMT variants was detected with this same 28 min program on HPLC.

When conducting kinetic experiments the K, was at least five fold higher than the minimum concentration of substrate and fivefold lower than the maximum concentration of substrate tested. Km and kcat were calculated by nonlinear regression to the Michaelis-Menten kinetics equation with the GraphPad PRISM 5.0 software.

### NMR

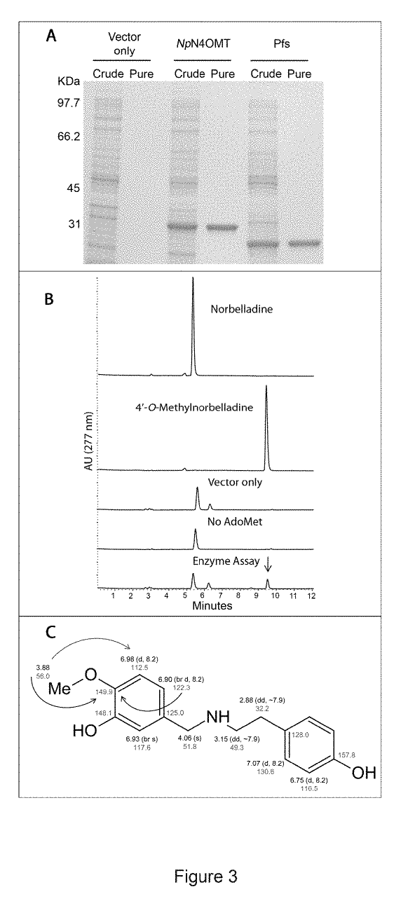

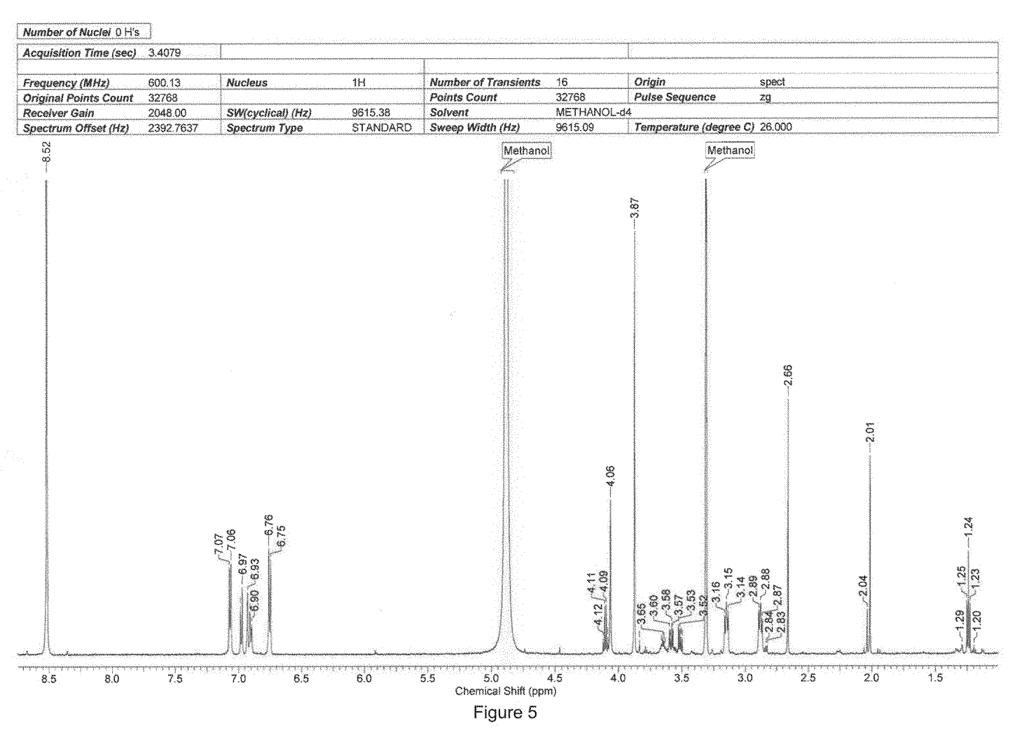

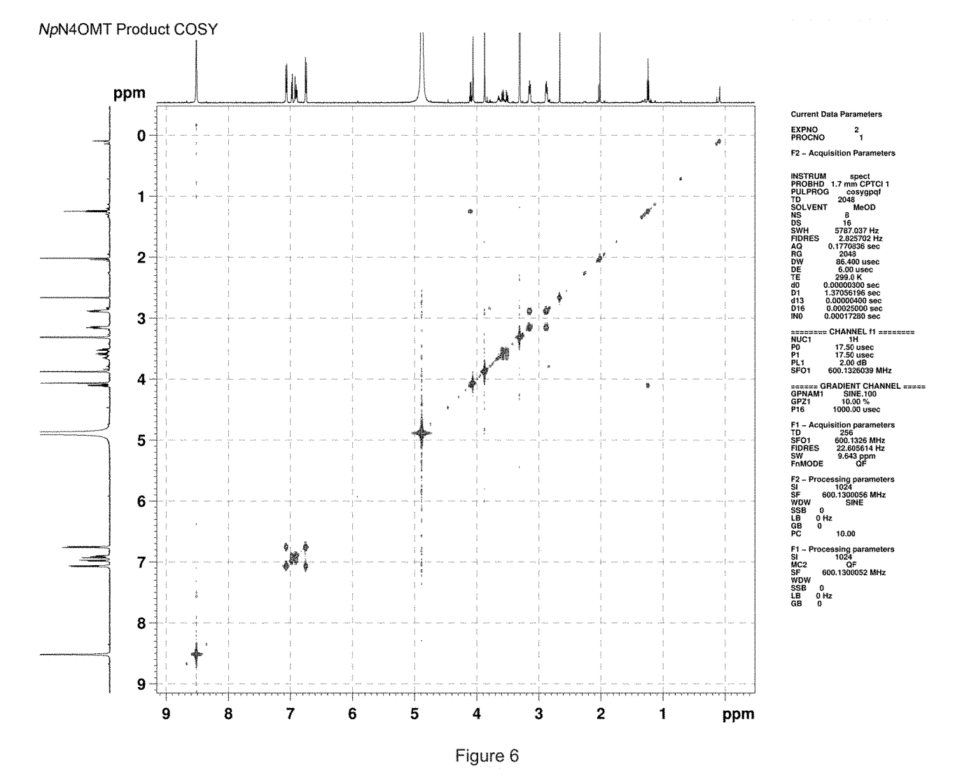

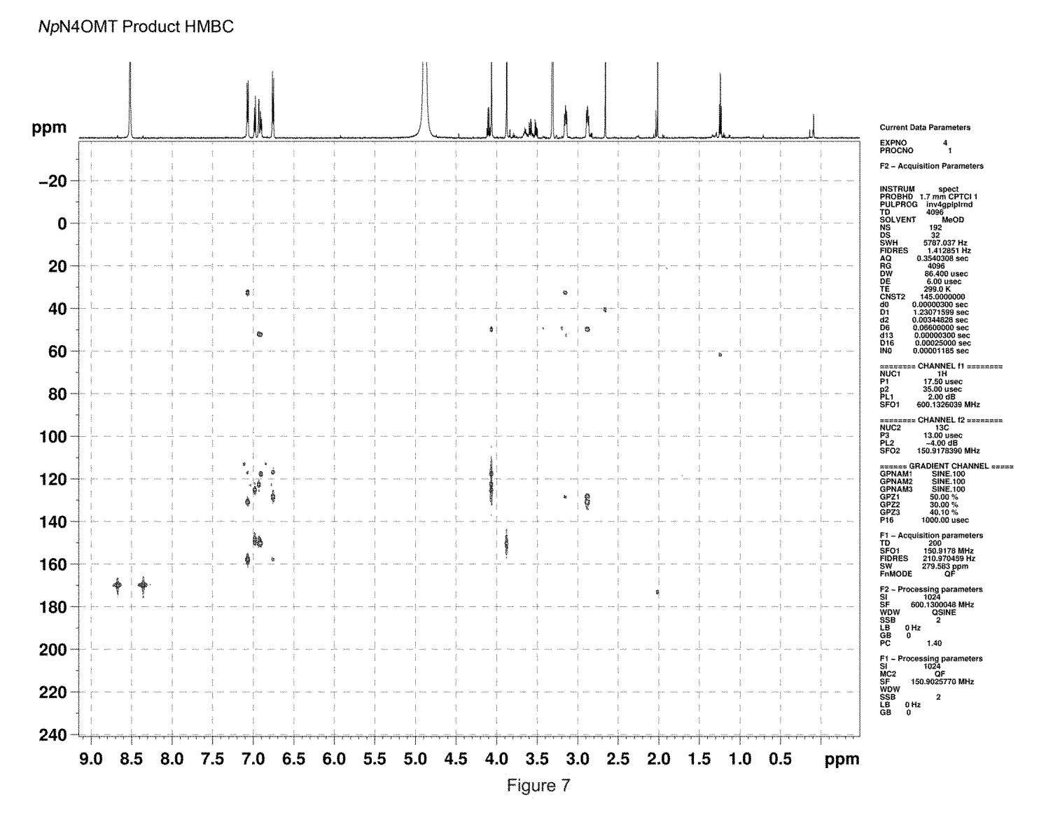

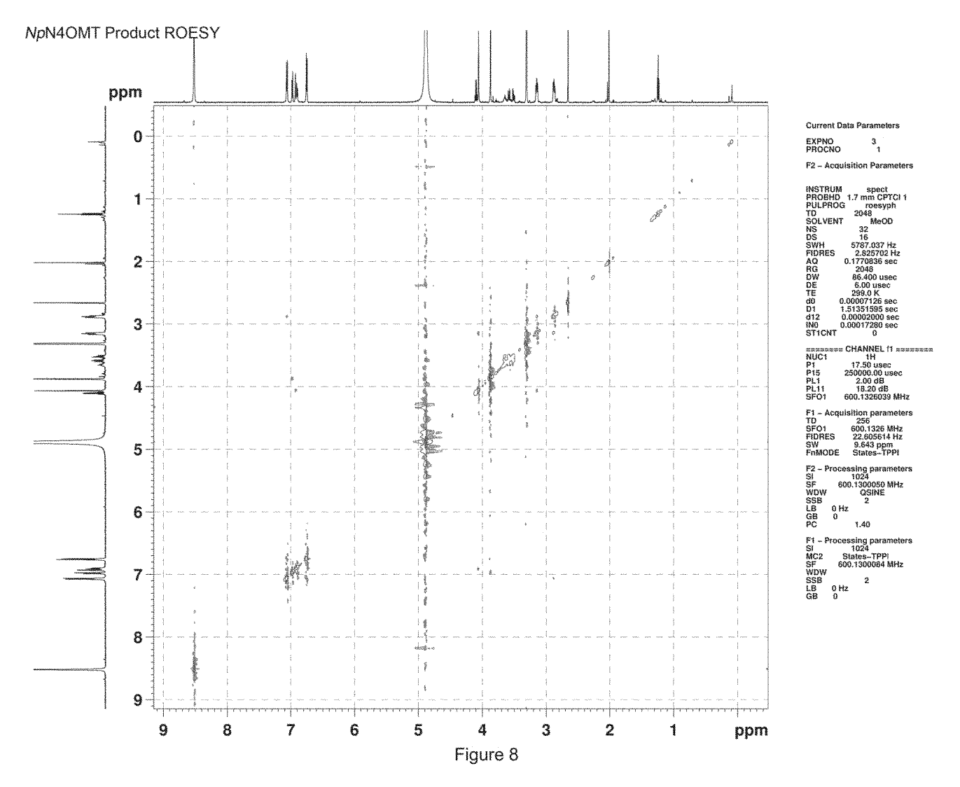

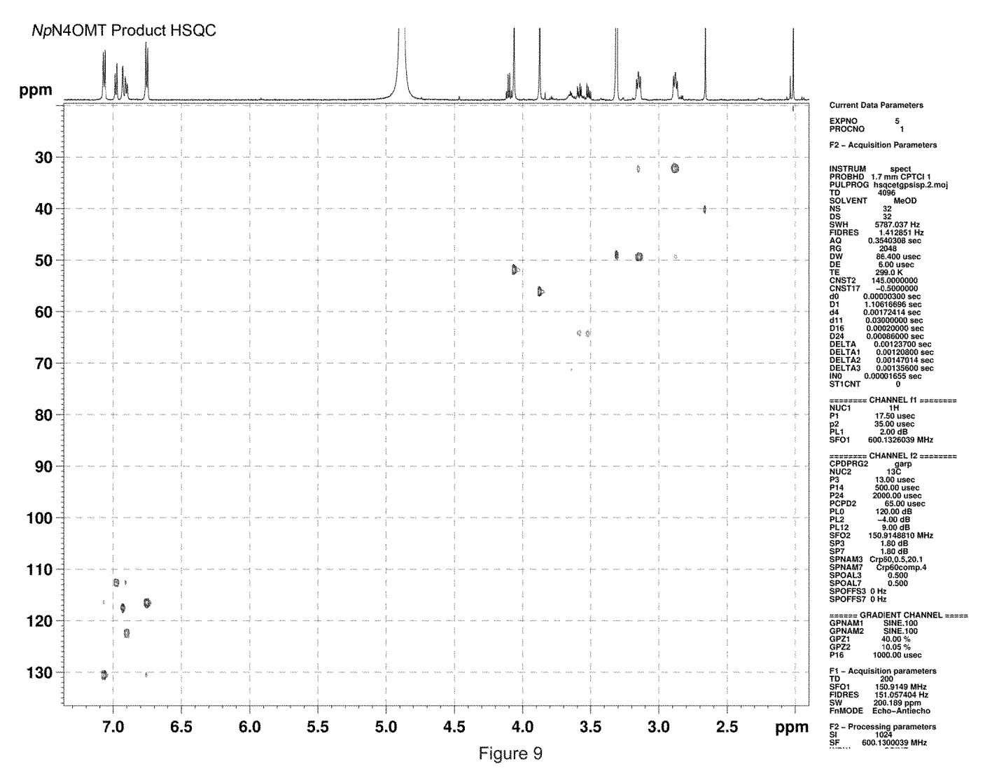

NMR spectra were acquired in CD₃OD at 600 MHz on a BrukerAvance 600 MHz spectrometer equipped with a BrukerBioSpin TCI 1.7 mm MicroCryoProbe. Proton, gCOSY, ROESY, gHSQC, and gHMBC spectra were acquired; ¹³C chemical shifts were obtained from the HSQC and HMBC spectra. Chemical shifts are reported with respect to the residual non-deuterated MeOD signal (FIGS. 5-9). Key chemical shifts for structure elucidation of 4′-O-methylnorbelladine are shown in FIG. 3C.

Quantitative Real Time-PCR (qRT-PCR)

cDNA for leaf, bulb and inflorescence tissues of daffodil were created using 1 μg RNA from the respective tissues, random primers and M-MLV reverse transcriptase according to the Invitrogen protocol. qRT-PCR was conducted with a TaqMan designed gene expression assay for the methyltransferase with ribosomal RNA as a reference according to manufacture protocol. Reactions (5 μl) were performed in quadruplicate with outlier exclusion using Applied Biosystems StepOnePlus Real-Time PCR system. Methyltransferase relative expression values were determined by calculating ΔΔC_{T} values relative to standard ribosomal RNA and leaf tissue.

### Results

The Illumina sequencing of Narcissus spp. leaf, bulb and inflorescence tissues resulted in 65 million paired reads that were used to make the Narcissus spp. transcriptome assembly. The transcriptome assembly consisted of 106,450 sequences with a mean length of 551 base pairs and a maximum length of 13,381 base pairs. A similar number of >100 base pair sequences were found in the transcriptome of Chlorophytum borivilianum. This mean length indicates a high number of the sequences are long enough for homology searches and cloning work. Of these sequences, 79,980 were predicted to have open reading frames and were translated into peptides. After determining the reads coming from the three tissues, several homologs of genes with predictable expression patterns were used to evaluate the quality of the expression estimations. The RuBisCO large and small subunits have high amounts of expression in the photosynthetic leaf and inflorescence tissues compared to the non-photosynthetic bulb tissue. A homolog to the MADS62 floral development transcription factor is exclusively expressed in the inflorescence tissue as would be expected. The read counts were thus determined to produce expected expression patterns.

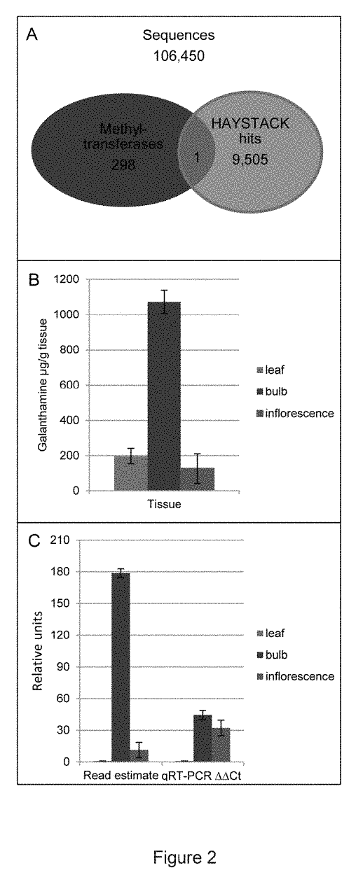

The LC/MS/MS data for leaf, bulb, and inflorescence tissues resulted in a pronounced accumulation pattern of galanthamine. The largest concentration was found in bulb tissue, with a lower level found in leaf and the lowest level in inflorescence (FIG. 2B).

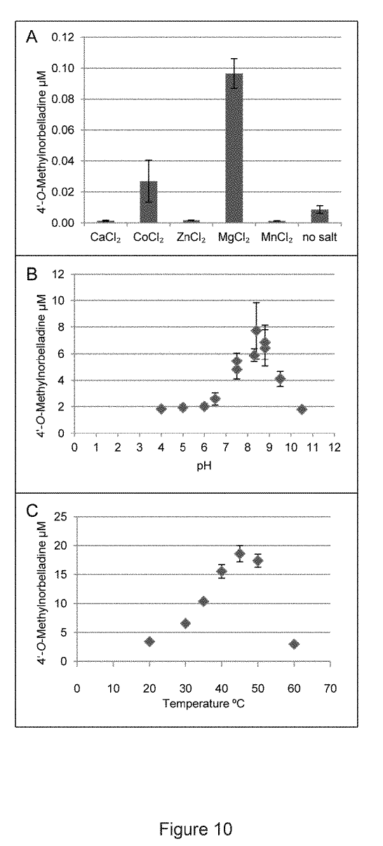

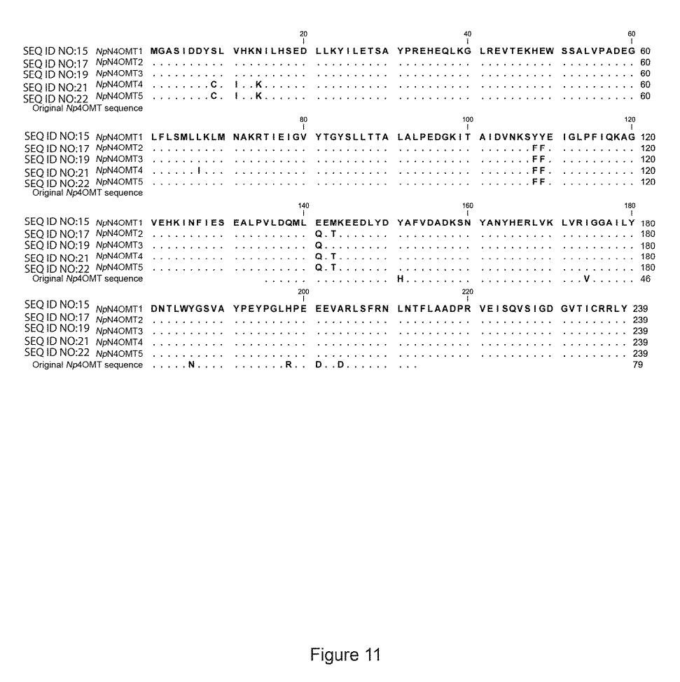

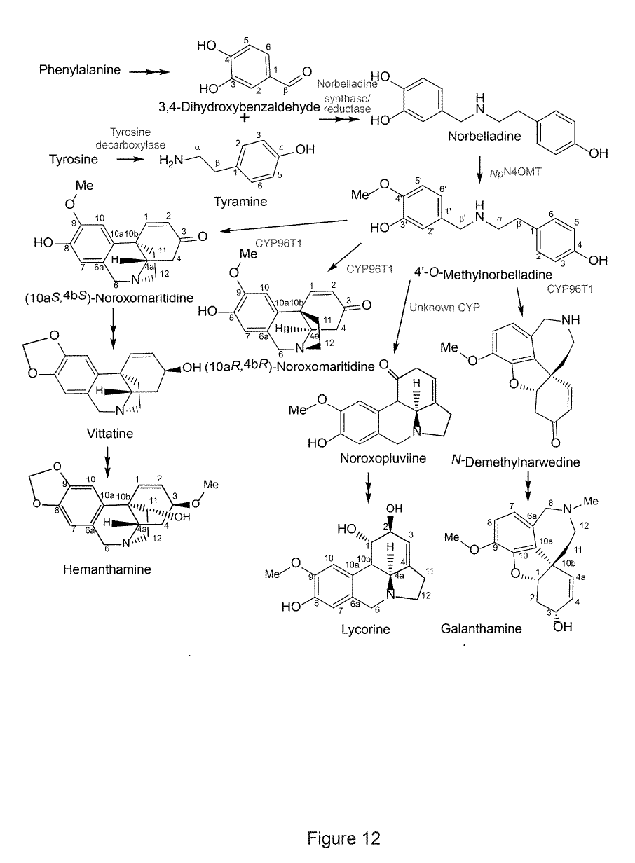

Using BLAST to seek homologs to the methyltransferases found in Table 1 yielded 298 methyltransferase candidate genes. Separately, HAYSTACK identified 9,505 contigs that co-express with galanthamine accumulation. A comparison of the two resulting lists revealed one methyltransferase, NpN4OMT, that fits the HAYSTACK model (FIG. 2A). This methyltransferase was chosen for functional analysis. After RACE, NpN4OMT was found to be a 239 amino acid protein with a predicted molecular weight (MW) of 27 kDa. When this protein was expressed using the pET28a vector, the added N-terminal Histidine tag increased the MW to 29 kDa (FIG. 3A). In the course of cloning, 5 unique clones were obtained with >96% identity to each other. Due to the two toned yellow flower color, single flower and size, the daffodil variety used in this study is likely Carlton. Based on genome size estimates, Carlton is suspected to be a domesticated form of Narcissus pseudonarcissus with a genome duplication that resulted in a tetraploid. A high number of paralogs is, therefore, expected. In addition, these bulbs have been propagated vegetatively. For these reasons the existence of so many similar sequences is not surprising. Due to the high similarity of the NpN4OMT clones, the first to be cloned was selected for thorough characterization. The clone selected for characterization is 92.5% identical on the amino acid level to the original sequence in the transcriptome assembly (FIG. 11). The recombinant protein was purified with a yield of 16.7 mg protein/L E. coli culture. SDS-PAGE analysis revealed the protein to be of apparent homogeneity (FIG. 3A). Initial enzyme assays with NpN4OMT1 yielded, upon HPLC analysis, a peak with the retention time of 4′-O-methylnorbelladine. The vector only control lacks NpN4OMT but has all other assay components. Therefor the absence of product in the vector control assay excludes the possibility of a background reaction. The absence of product in the assay lacking AdoMet shows that the methyltransferase uses AdoMet as a co-substrate and cannot form product without AdoMet (FIG. 3B). The pH optimum was found to be 8.8 and the temperature optimum 45° C. (FIG. 10B-C).

An alternative methylation product, 3′-O-methylnorbelladine, has the same retention time on HPLC, the same UV profile and MS/MS fragmentation pattern as 4′-O-methylnorbelladine. Thus, NMR analysis was performed to determine the regiospecificity of O-methylation. HMBC correlations from both the methoxyl protons (δ_{H} 3.88) and H-6′ (δ_{H} 6.90) to the same carbon (δ_{C} 149.9) placed the methoxyl group at C-4′. Its location was further supported by a ROESY correlation from the methoxyl protons to H-5′ (δ_{H} 6.98). The NMR data thus confirmed that 4′-O-methylnorbelladine is the product of the enzyme reaction (FIG. 3C).

To determine the substrate specificity of this methyltransferase, we tested several similar substrates. The results are shown in Table 7.

TABLE 7
Substrate specificity of NpN4OMT1

| | | | k_{cat}/Kₘ |
|---|---|---|---|
| Substrate | Kₘ (μM) | k_{cat} (1/min) | (1/μM*min) |
| norbelladine | 1.6 ± 0.3 | 1.3 ± 0.06 | 0.8 |
| AdoMet | 28.5 ± 1.6 | 4.5 ± 0.01 | 0.16 |
| N-methylnorbelladine | 1.9 ± 0.4 | 2.6 ± 0.15 | 1.3 |
| dopamine | 7.3 ± 2.7 | 3.6 ± 0.15 | 0.5 |
| caffeic acid | ND | ND | ND |
| vanillin | ND | ND | ND |
| 3,4- | ND | ND | ND |
| dihydroxybenzaldehyde | | | |
| tyramine | ND | ND | ND |

ND = Not detected
± = Standard error

Activity comparable to that found with norbelladine was observed using N-methylnorbelladine as the NpN4OMT substrate. Dopamine also served as a substrate, but with less efficiency. Products were not detected when testing caffeic acid, vanillin, 3,4-dihydroxybenzaldehyde, and tyramine as substrates. To determine if the other 4 variants show similar activity, they were purified, and enzymatic activity was confirmed for all variants using norbelladine as the substrate. When monitoring NpN4OMT norbelladine assays allowed to proceed to completion, no sign of double methylation products were observed as expected.

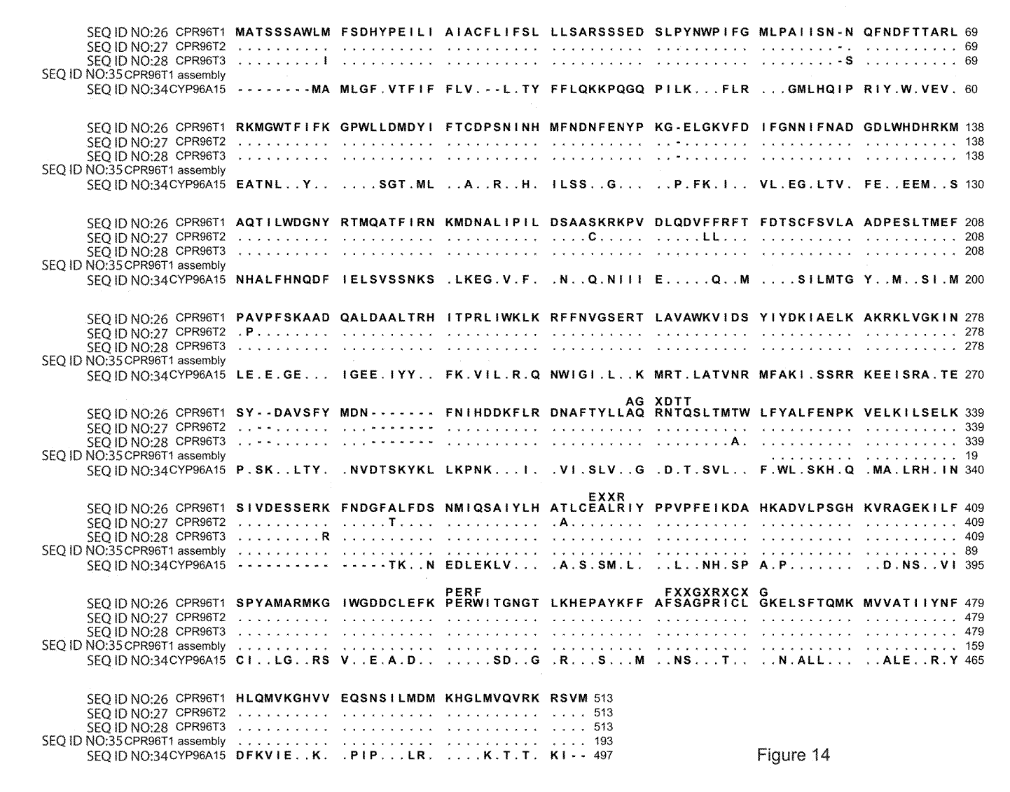

The pattern-matching algorithm HAYSTACK was used to identify transcripts that co-express with N4OMT when searching for a cytochrome P450. N4OMT is the only validated gene involved in Amaryllidaceae alkaloid biosynthesis to date. Its position in the pathway is just prior to the C—C phenol-coupling step therefore, N4OMT gene expression is a logical choice to serve as a model for analysis of co-expressing transcripts encoding additional Amaryllidaceae alkaloid biosynthetic genes. Since the C—C phenol-coupling enzyme is targeted herein, BLASTP was used to find transcripts that encode putative cytochrome P450 enzymes. The resulting 544 daffodil cytochrome P450 protein sequences were compared to the list of 3,704 N4OMT co-expressing transcripts identified by HAYSTACK. This resulted in the identification of 18 N4OMT co-expressing cytochrome P450 transcripts in the daffodil assembly. The Galanthus assemblies were interrogated using these 18 sequences to identify close homologues. This allowed for selection of the cytochrome P450 transcripts that consistently co-expressed with N4OMT across species in all assemblies. One candidate (CYP96T1) co-expressed with N4OMT in all assemblies and was investigated further. A close homologue to CYP96T1 with 99% identity in shared ORF sequence and the first 67 bases of the 3′ UTR was identified. In contrast to CYP96T1, this transcript was complete at the 5′ end of the ORF and contained 5′ UTR sequence information. This allowed the incomplete 5′ region of CYP96T1 to be predicted by comparison. The PCR product generated with outer primers was sequenced and the inner primer sequences were found not to deviate from the assembly prediction. A clone was acquired with no conflicts to the previously known CYP96T1 sequence and was used for functional characterization. Two additional variants were cloned reproducibly. The closest biochemically characterized homologue to CYP96T1 was CYP96A15 from Arabidopsis thaliana (Q9FVS9) (FIG. 14).

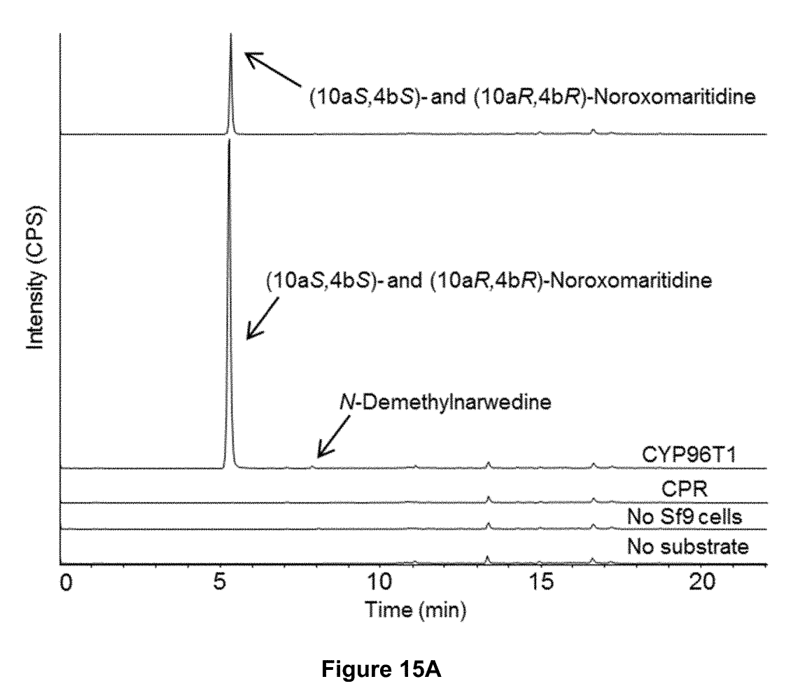

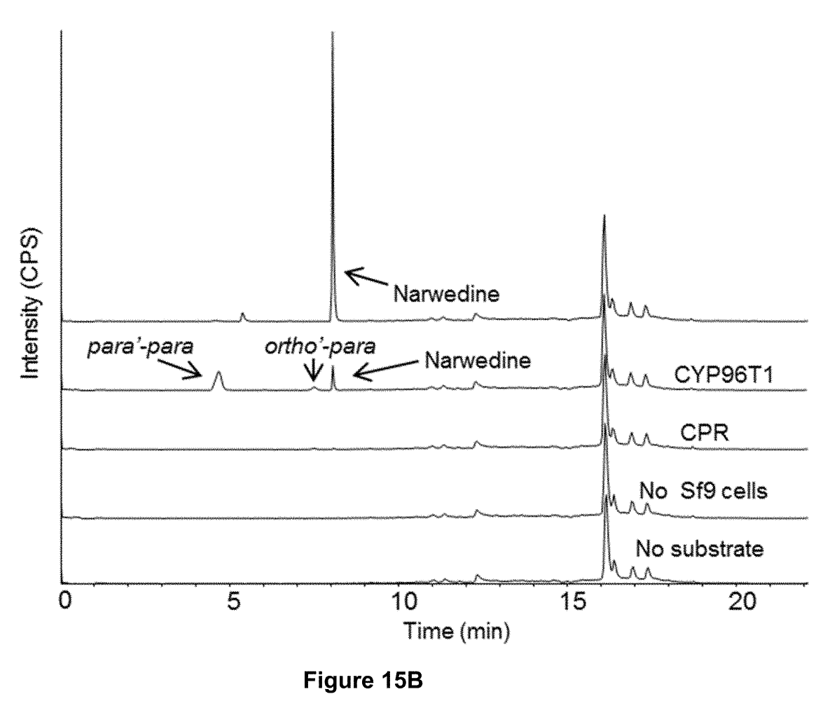

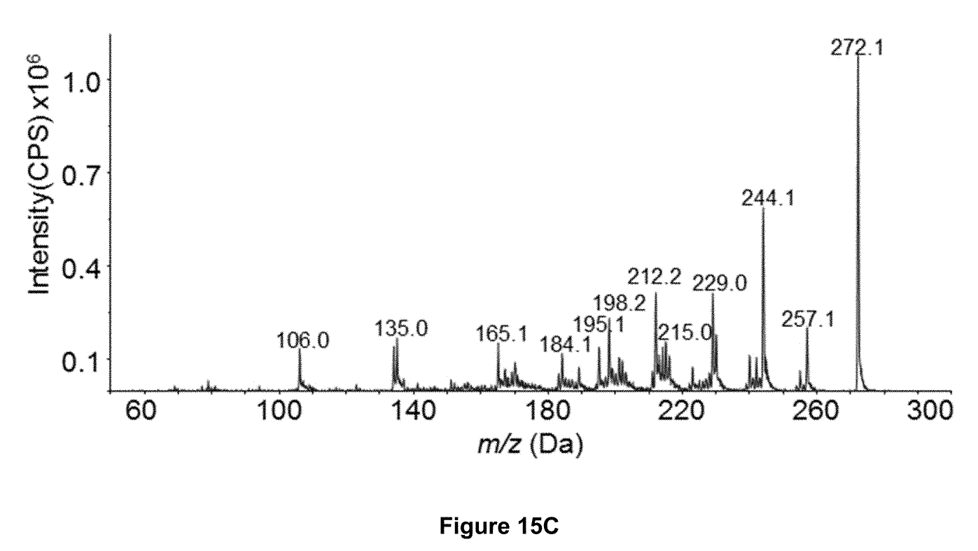

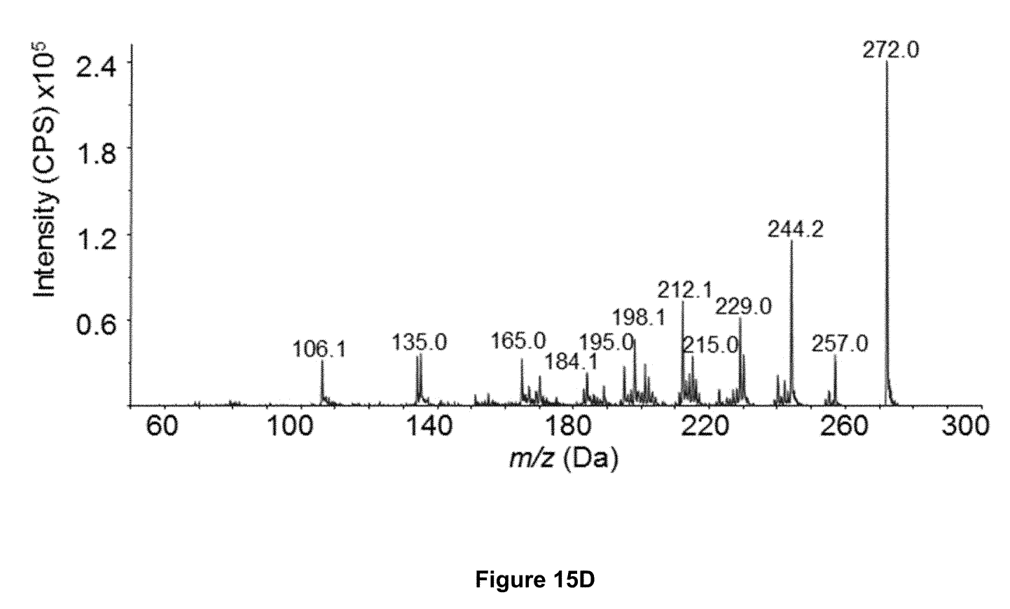

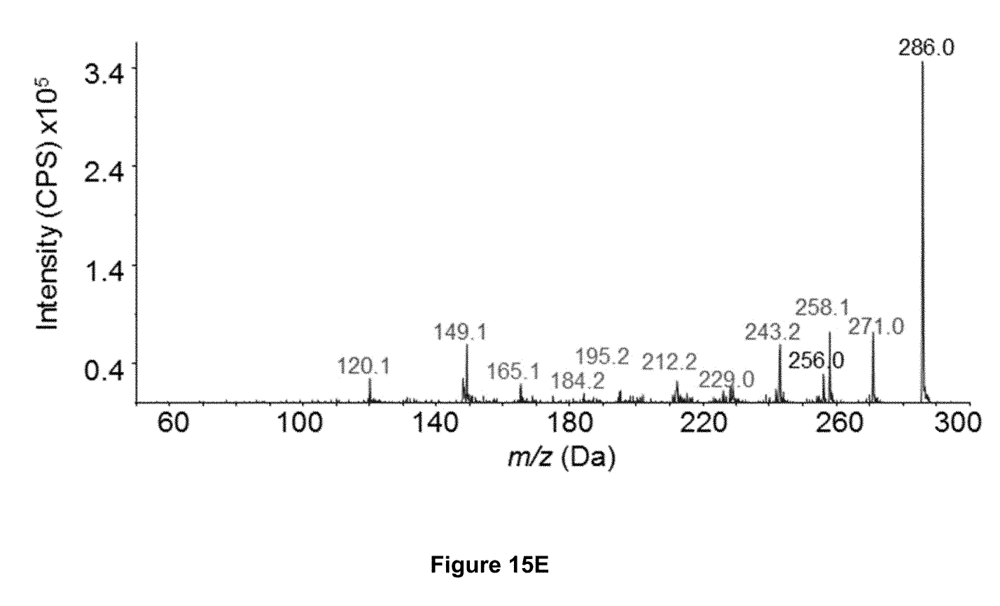

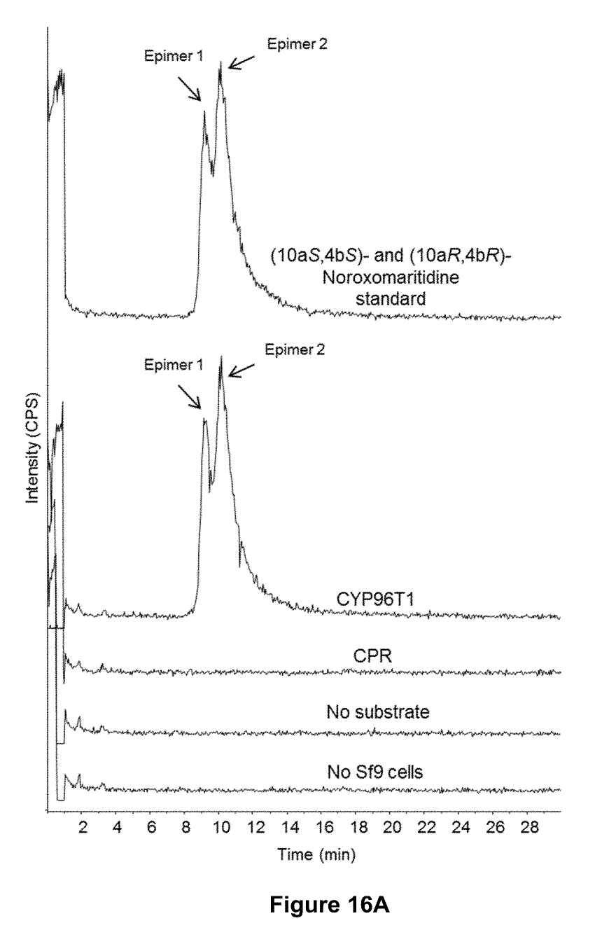

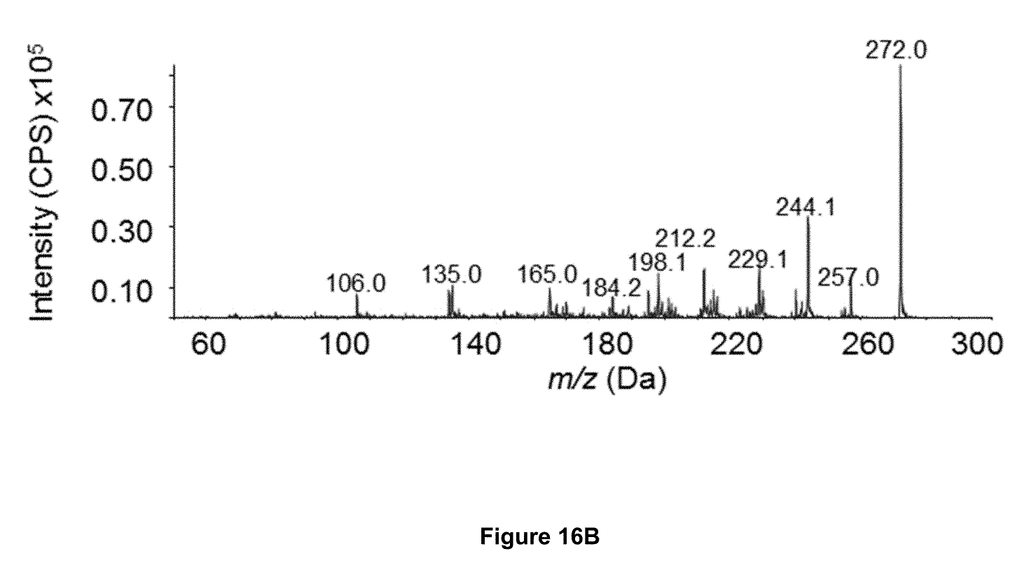

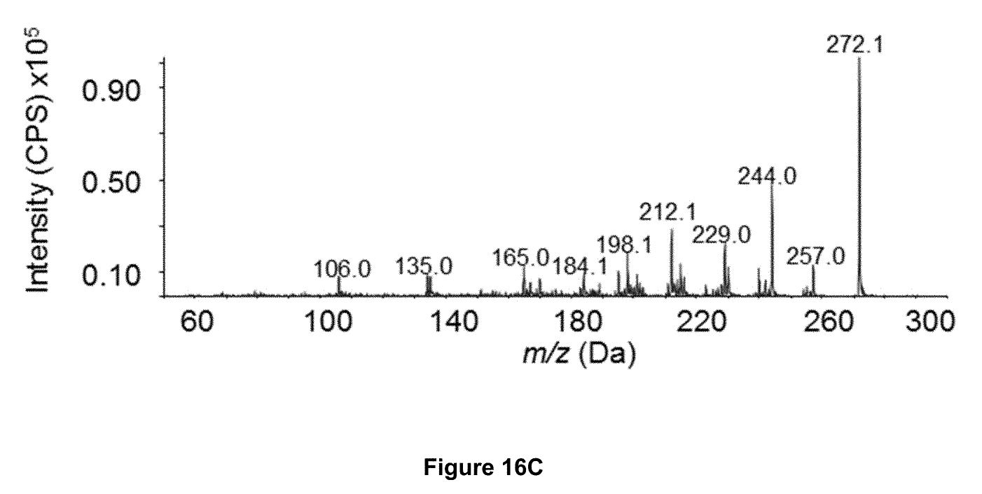

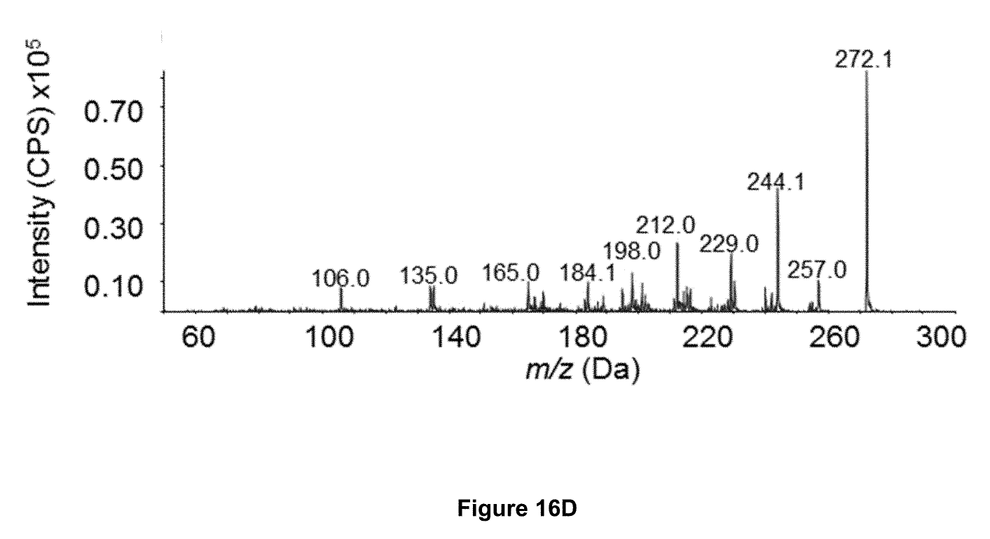

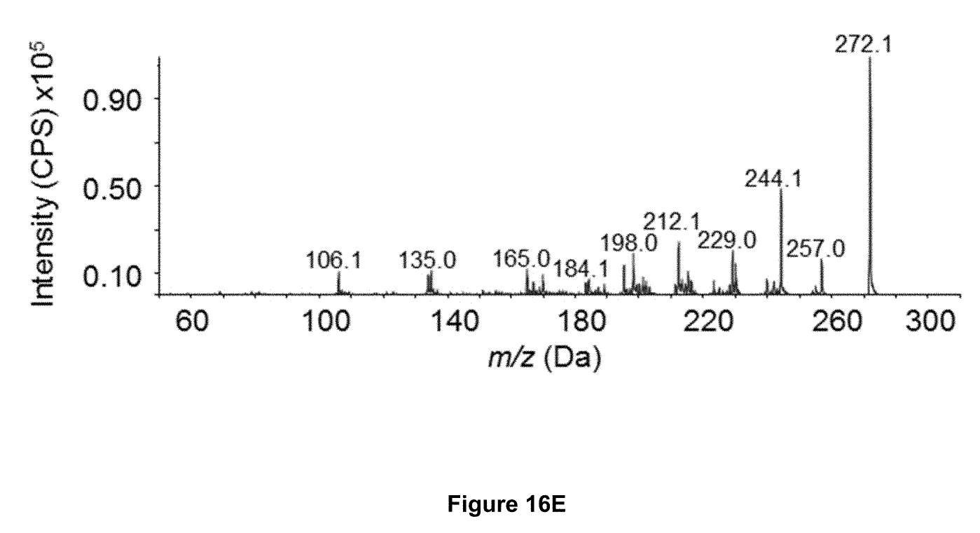

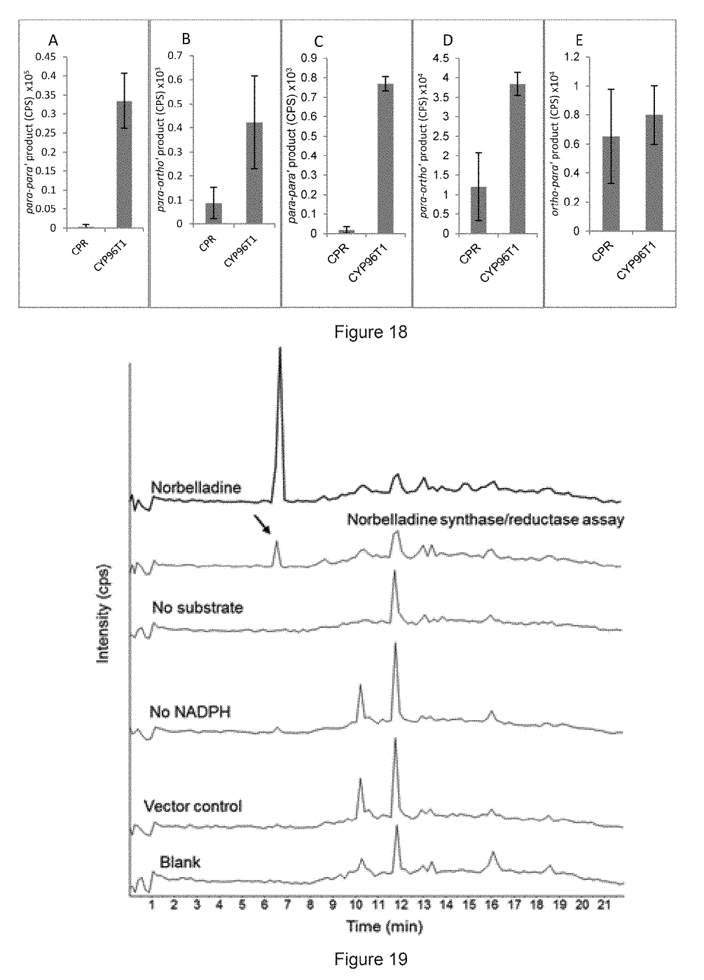

The concentration of CYP96T1 in Sf9 cell culture was determined to be 2.5 nM by CO-difference spectra. The temperature and pH optima for 4′-O-methylnorbelladine substrate were determined to be 30° C. (half height+5-10° C.) and 6.5 (half height+1), respectively. Testing of the CYP96T1 enzyme demonstrated that several structurally related alkaloids were C—C phenol coupled as detected by LC-MS/MS. These reactions were accompanied by a background reaction catalyzed by the Sf9 cells. 4′-O-methylnorbelladine was C—C phenol coupled into N-demethylnarwedine, (10aR,4bR)- and (10aS,4bS)-noroxomaritidine in CYP96T1 assays. (10aR,4bR)- and (10aS,4bS)-noroxomaritidine was identified by its identical liquid chromatographic retention time (FIG. 15 A) and mass spectrometric fragmentation pattern with (10aR,4bR)- and (10aS,4bS)-noroxomaritidine mixed standard (FIGS. 15 C and D) (Table 8). To determine the chirality of the noroxomaritidine product, 4′-O-methylnorbelladine assays with CYP96T1 were analyzed with a chiral-CBH column by LC-MS/MS. Chromatographic separation of (10aR,4bR)- and (10aS,4bS)-noroxomaritidine standards was achieved preceding MS/MS analysis. All variants produced equivalent amounts of each epimer (FIG. 16 A). A mass spectrometric comparison of standards (FIGS. 16 B and C) and enzymatically formed (10aR,4bR)- and (10aS,4bS)-noroxomaritidine (FIGS. 16 D and E) yielded identical MS/MS fragmentation patterns. The enzyme is, therefore, producing both (10aR,4bR)- and (10aS,4bS)-noroxomaritidine. A minor N-demethylnarwedine product was also detected in assays analyzed by HPLC on the Luna C8 column. The relative quantity of (10aR,4bR)- and (10aS,4bS)-noroxomaritidine and N-demethylnarwedine formed in assays with CYP96T1 are quantified in FIGS. 18 A and B. HPLC was used to measure the relative contribution of these compounds to total product. (10aR,4bR)- and (10aS,4bS)-noroxomaritidine account for ˜99% of the total product in CYP96T1 assays. (10aR,4bR)- and/or (10aS,4bS)-noroxomaritidine and N-demethylnarwedine are also produced in assays containing only Sf9 cells and 4′-O-methylnorbelladine, but not in a no enzyme control, indicating Sf9 cells that have the ability to catalyze the C—C phenol couple with 4′-O-methylnorbelladine (FIG. 15 A). Kinetic analysis of the CYP96T1 production of (10aR,4bR)- and (10aS,4bS)-noroxomaritidine using nonlinear regression to the Michaelis-Menten kinetics equation for substrate inhibition show the K, to be in the low micro molar 1.13±0.5 μM with a k_{cat} of 15.0+2.03 l/min (Table 8). In addition, the N-methylated form of 4′-O-methylnorbelladine,4′-O-methyl-N-methylnorbelladine, was shown to produce several C—C phenol-coupled products when assayed with Sf9 cells alone as indicated by the detection of products with a mass reduction of 2 m/z, including narwedine and two unknown products (FIG. 15 B). One product is enzymatically produced from 4′-O-methyl-N-methylnorbelladine by CYP96T1, as indicated by the increase of product in assays containing CYP96T1 as compared to the CPR-only control (FIG. 15 B). These observations were confirmed by a MRM based relative quantification of selected transitions of these three products (FIGS. 18 C, D and E). The LC-MS/MS fragmentation pattern of the CYP96T1 product is a mixture of masses found in the para′-para products (10aR,4bR)- and (10aS,4bS)-noroxomaritidine (165.1 m/z, 184.2 m/z, 195.0 m/z, 212.2 m/z, 229.0 m/z) and masses+14 m/z (120.1 m/z, 149.1 m/z, 243.2 m/z, 258.1 m/z, 271.0 m/z), representing the addition of a methyl moiety (FIG. 15 E). For this reason, it appears the enzyme is capable of catalyzing formation of the para-para′C—C phenol-couple regardless of N-methylation state. (FIGS. 18 D and E). To examine the ability of CYP96T1 to C—C phenol couple substrates with an altered carbon linker between the phenol groups, (S)-coclaurine and (R)-coclaurine were also tested. Assays on ether (S)-coclaurine or (R)-coclaurine yield products with a mass −2 m/z which is consistent with a C—C phenol coupling. Product formation is not observed when norbelladine or N-methylnorbelladine is used as substrate. These results indicate the 4′-O-methylation state of norbelladine may be important for substrate-enzyme binding. The substrates 3′-O-methylnorbelladine and 3′,4′-O-dimethylnorbelladine were tested to determine the relevance of 3′-O-methylation; products were not detected (Table 8).

TABLE 8
Substrate specificity tests for CYP96T1

| | | | K_{cat}/Kₘ | | Modifications |
|---|---|---|---|---|---|
| Substrate | Kₘ (μM) | k_{cat} (1/min) | (1/μM*min) | Activity | monitored |
| 4′-O- | 1.13 ± 0.5 | 15.0 ± 2.03 | 13 | + | C-C phenol |
| Methylnorbelladine | | | | | coupling |
| 4′-O-Methyl-N- | Undetermined | Undetermined | Undetermined | + | C-C phenol |
| methylnorbelladine | | | | | coupling |
| (S)-Coclaurine | Undetermined | Undetermined | Undetermined | + | Intramolecular |
| | | | | | phenol |
| | | | | | coupling and |
| | | | | | Intermolecular |
| | | | | | coupling |
| (R)-Coclaurine | Undetermined | Undetermined | Undetermined | + | Intramolecular |
| | | | | | phenol |
| | | | | | coupling and |
| | | | | | Intermolecular |
| | | | | | coupling |
| 3′-O- | NA | NA | NA | ND | C-C phenol |
| Methylnorbelladine | | | | | coupling |
| 3′,4′-O- | NA | NA | NA | ND | C-C phenol |
| Dimethylnorbelladine | | | | | coupling |
| Norbelladine | NA | NA | NA | ND | C-C phenol |
| | | | | | coupling |
| N- | NA | NA | NA | ND | C-C phenol |
| Methylnorbelladine | | | | | coupling |
| Haemanthamine | NA | NA | NA | ND | Methoxy |
| | | | | | bridge |
| | | | | | formation and |
| | | | | | hydroxylation |
| (10aR,4bR)- and | NA | NA | NA | ND | Methoxy |
| (10aS,4bS)- | | | | | bridge |
| Noroxomaritidine | | | | | formation and |
| | | | | | hydroxylation |
| Isoyanillin and | NA | NA | NA | ND | C-C phenol |
| tyramine | | | | | coupling, |
| | | | | | amine- |
| | | | | | aldehyde |
| | | | | | condensation, |
| | | | | | amine- |
| | | | | | aldehyde |
| | | | | | condensation |
| | | | | | and C-C |
| | | | | | phenol |
| | | | | | coupling |

ND = not detected
NA = not applicable

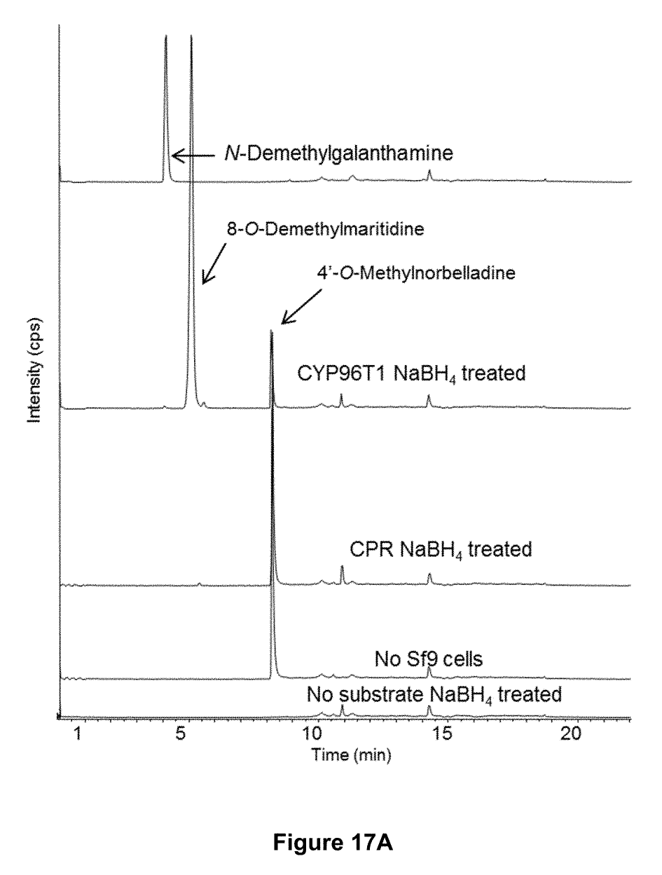

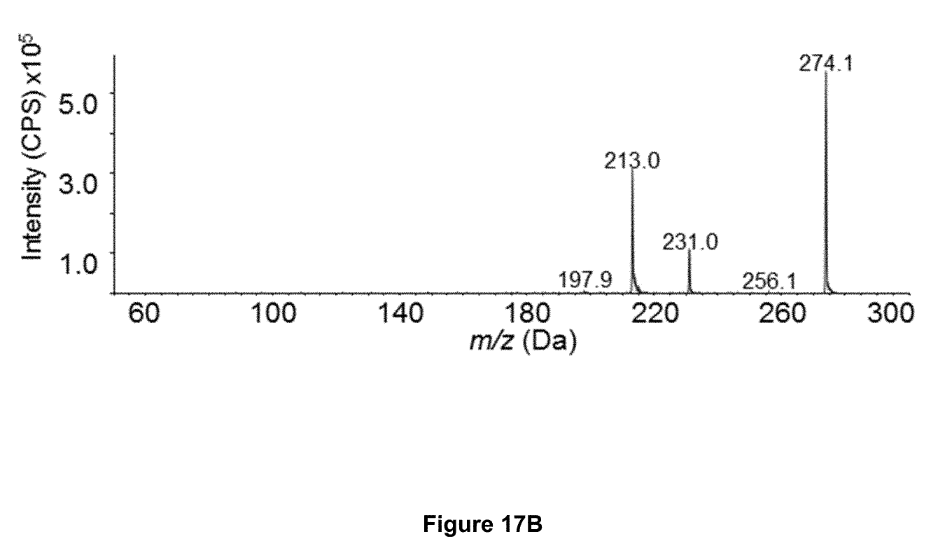

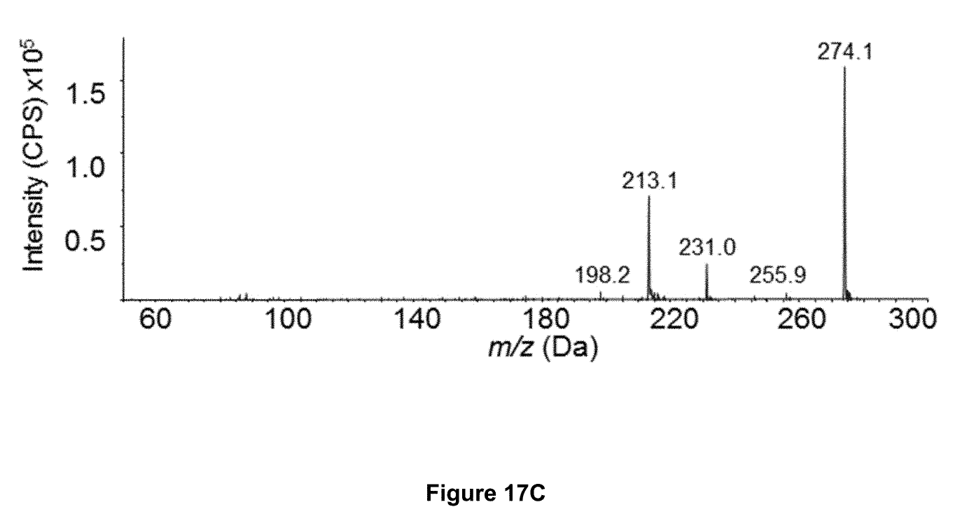

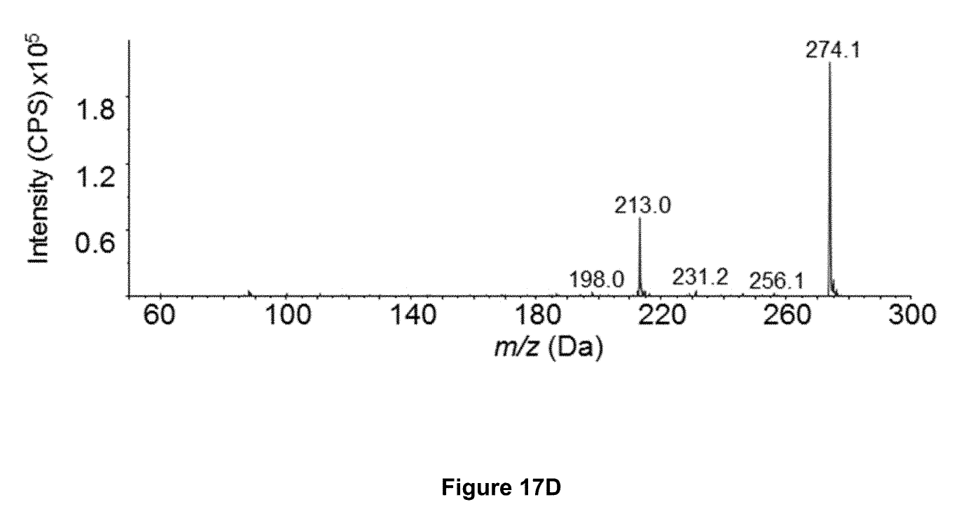

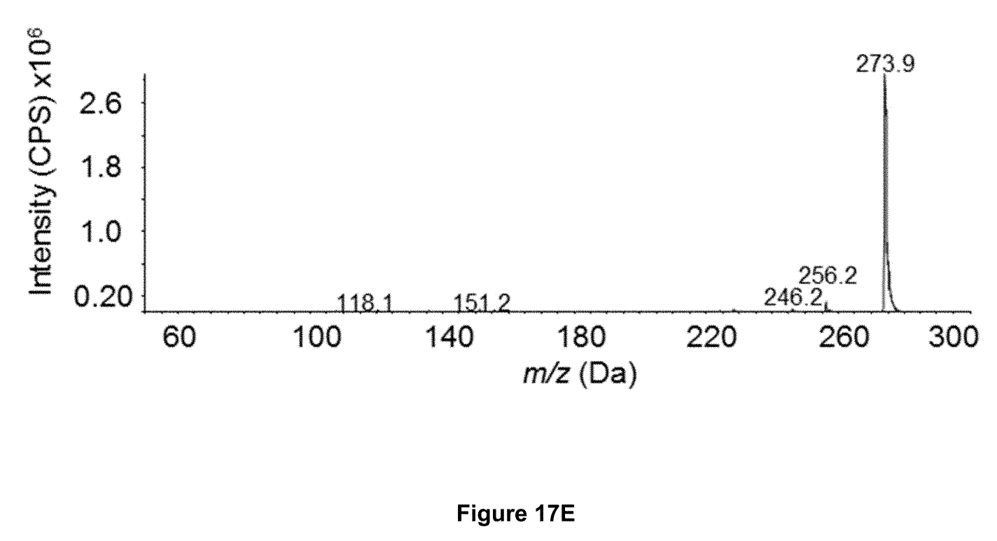

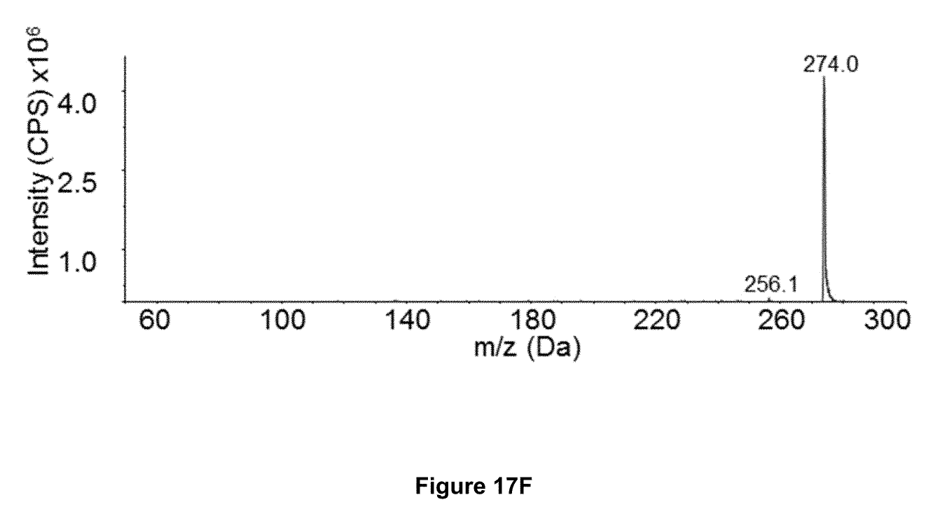

Enzymatically formed N-demethylnarwedine from enzyme assays with CYP96T1 was converted to N-demethylgalanthamine by sodium borohydride reduction and detected by LC-MS/MS (FIG. 17 A). Sodium borohydride selectively reduced the ketone group on (10aR,4bR)- and (10aS,4bS)-noroxomaritidine and N-demethylnarwedine to yield a stereoisomeric mixture of the corresponding alcohols 8-O-demethylmaritidine and N-demethylgalanthamine. Confirmation of N-demethylgalanthamine in these assays is demonstrated by the identical retention time (FIG. 17 A,) and fragmentation pattern (FIGS. 17 B, and C) with N-demethylgalanthamine standard. Another peak is also present with a different retention time (FIG. 17 A) and very similar fragmentation pattern (FIG. 17 D) and is likely the diastereomer epi-N-demethylgalanthamine formed by non-stereospecific ketone reduction. Stereoisomeric 8-O-demethylmaritidine is present in sodium borohydride reduced CYP96T1 4′-O-methylnorbelladine assays as the largest product peak (FIG. 17 A). This is validated by a comparison of the LC-MS/MS fragmentation pattern of (10aR,4bR)- and (10aS,4bS)-noroxomaritidine reduced by sodium borohydride to the corresponding peak in the CYP96T1 assay (FIGS. 17 E and F).

Norbelladine synthase/reductase assays have increased production of norbelladine compared negative controls lacking substrate, co-substrate or enzyme as shown in figure (FIG. 19).

Phylogenetic analysis of the NpN4OMT1 placed it in the class I OMT group. NpN4OMT1 has a length consistent with the 231-248 amino acid range found in class I OMTs. This is in contrast to other known plant catechol 4-OMTs, which all group in the class II OMTs as their length and cofactor requirements reported in previous work would predict. All these methyltransferases are significantly longer than the standard class I OMTs and none is reported to have the characteristic divalent cation dependence of class I OMTs. When testing NpN4OMT1 for cation dependence, enzymatic activity improved upon the addition of cobalt. Enzymatic activity increased fourfold more with the addition of magnesium instead of cobalt (FIG. 10A). This preference for magnesium over other divalent cations is also to be expected from a class I OMT. It is, furthermore, consistent with previous work on enzyme extracts enriched for this OMT.

To validate the expression profiles predicted based on read counts for NpN4OMT; qRT-PCR was conducted with the same RNA preparation used to prepare the cDNA libraries for Illumina sequencing. The resulting expression profile is slightly different from that obtained from Illumina sequencing. The qRT-PCR expression profile has a higher quantity of inflorescence transcript relative to bulb transcript (FIG. 2C). This minor difference is potentially due to cross amplification, during qRT-PCR, with other close homologs in the plant.

### DISCUSSION

The expression pattern, product formation and low Kₘ for norbelladine all indicate that NpN4OMT methylates norbelladine in the galanthamine biosynthetic pathway. Two differing orders of methylation have been proposed for galanthamine biosynthesis. The methylation of N-methylnorbelladine was tested to determine if a preference for the N-methylation state could be observed at O-methylation. Similar Kₘ and k_{cat} values for N-methylnorbelladine and norbelladine indicate that a preference for the N-methylation state does not occur at O-methylation. The results presented here support both proposed galanthamine biosynthetic pathways. Future work on additional enzymes in the pathway will be needed to enzymatically validate one pathway or the other. The lack of enzymatic activity when testing 3,4-dihydroxybenzaldehyde suggests that methylation does not occur prior to formation of norbelladine. The methylation of dopamine is expected considering structural similarity to the methylated moiety of norbelladine. Tyramine was not methylated; this is as expected for a class I OMT.

Several aspects of the candidate gene selection approach proved important for this successful identification. These aspects include but are not limited to: (1) selection of methyltransferases for the homology search; (2) expression in direct relationship to galanthamine accumulation; (3)

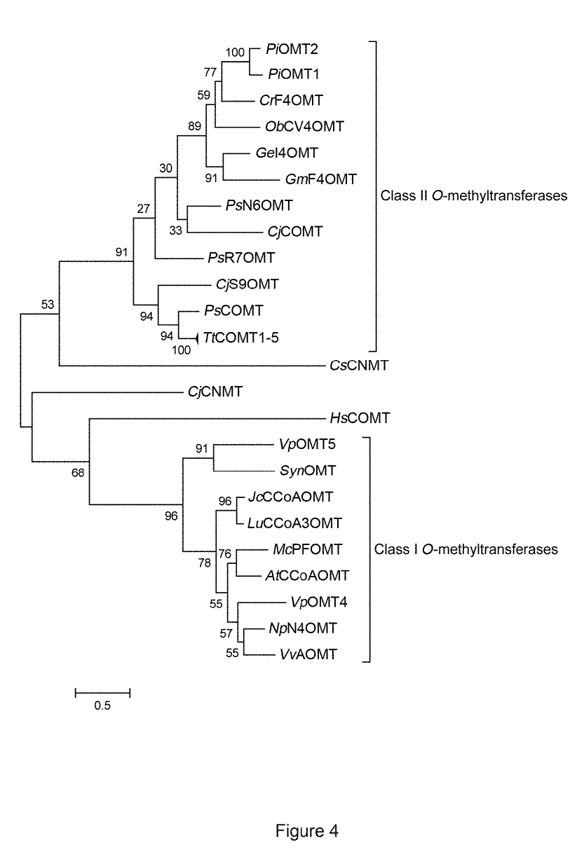

One is the selection of a variety of methyltransferases for the homology search. If only the known 4-OMTs had been used in the homology search, the gene identified in this example would have been missed due to the large difference in sequence between known 4-OMTs and NpN4OMT. It has been shown that one amino acid can be the difference between a catechol 4′-OMT and a 3′-OMT. Because of this potential for a conversion from catechol 3′-O-methylation to 4′-O-methylation though evolution, OMTs of both positions were used in the homology search. Also, both class I and class II OMTs were used in the search because both classes are known to methylate catechols. Considering the multiple branches of the N-methyltransferases off the OMT phylogeny, it is worth investigating enzymes that annotate as N-methyltransferases. For these reasons, the sequences used in the initial BLAST search consisted of representatives of known 0- and N-methyltransferases of small metabolites. The NpN4OMT turned out to be a member of the class I OMTs. Class I OMTs show closer homology to the human catechol OMT than to all known plant catechol 4-OMTs that are in the class II OMTs as demonstrated in FIG. 4. The closest known catechol 4-OMT to NpN4OMT is bacterial, has 34% identity to NpN4OMT, and is a class I OMT from Cyanobacterium Synechocystis sp. Strain PCC 6803 (SynOMT). Many 3-OMTs show even higher homology to NpN4OMT than SynOMT. It is probable that the 4-OMT activity of NpN4OMT was acquired independently of SynOMT (FIG. 4).

The second selection criterion, co-expression with galanthamine accumulation, was also of great value. It reduced the number of candidate OMTs from hundreds to one. There are a variety of methods for the prioritization of candidate genes [54,55]. Many of these methods are oriented towards species and systems for which there are extensive databases or prior knowledge regarding a gene involved in the pathway or process. In one study, a collection of −500 microarray files was used to demonstrate the co-expression of genes in the same pathway in Arabidopsis. However, this vast number of microarrays is not available in non-model systems that have not been as thoroughly studied as Arabidopsis. There have been several studies that use co-expression analysis to find genes in a pathway and produce promising candidate genes lists. These studies sometimes stopped with in silico candidates without in vitro validation of enzymatic activity. If there is a novel function proposed, this type of analysis is incomplete without biochemical validation. Enzymes that are homologous to functionally equivalent enzymes in a different species can be validated by co-expression analysis. There are several good studies that use a simple differential expression model and microarrays to find biosynthetic genes by comparing biosynthetically active and inactive accessions in rose and strawberry. Differential expression analysis lacks the means to use data with differing levels of metabolism occurring in more than 2 samples. The Pearson correlation used in this study can handle data from multiple samples. Mercke et al. have used a Pearson correlation-based method to identify genes that correlate with levels of specific terpenes in cucumber. In that study, microarrays were constructed instead of creating a transcriptome with Illumina sequencing. Illumina-based transcriptomes are more sensitive to minor variants in the sequences and to splice variants. Illumina-based gene expression data also have a far greater dynamic range, limited by sequence depth, than microarrays. Subtleties in the sequences that could be missed with microarrays can now be detected with Illumina sequencing.

The use of HAYSTACK as a platform to use the Pearson correlation is ideal because it is designed to receive a hypothesis for gene expression and look for genes that correlate with that hypothesis. This is in contrast to an approach in which genes are clustered based on similarity to each other. The search for a very particular pattern in the data allows the number of required expression data points to be reduced compared to an approach that needs to define clusters of genes based on shared expression patterns. In HAYSTACK, the shared expression pattern is already defined. HAYSTACK applies additional screening criteria including a p-value test for significance, a fold cut off and background cutoff. The approach chosen in our study used knowledge of known chemical intermediates, a transcriptome with expression profiles for three tissues, and metabolite levels to identify a candidate gene to validate with in vitro activity. Little prior knowledge of a pathway is required to use this approach, making this workflow ideal for the identification of genes in a biochemical pathway.

To discover this NpN4OMT several obstacles needed to be overcome and ambiguities clarified. Examples of such obstacles include but are not limited to: First, the substrates norbelladine and N-methylnorbelladine tested in this paper are not available in chemical catalogs but were synthesized in the lab. Second, when the study was started the exact location of galanthamine synthesis was unknown. The hypothesis is that biosynthesis is reflected in the accumulation of product. However, there are known cases in secondary metabolism where this is not the case. The compound could have been transported to its current location as in Nicotine biosynthesis. Galanthamine could have only just started or stopped being synthesized in some tissues. This would lead to galanthamine accumulation levels that are an indication of past biosynthesis rather than current biosynthesis. Third, the choice of methyltransferases to use when looking for candidates with BLAST was not straight forward. The choice had to be made to include all OMTs. If 4-OMTs had only been used the similarity to NpN4OMT may not have been high enough for its identification.

There are several modifications to this approach that could be used to improve its power. It could be applied to more tissues, environmental conditions, or time points to provide even greater statistical power to correlate co-expression of biosynthetic genes with the biosynthesis of their products. It could also be modified to include analysis of product accumulation in related pathways. The need for a particular enzyme is not necessarily dependent on one product. If the pathway the enzyme is in splits downstream, several end products could be equally important when doing co-expression analysis. This combined consideration of multiple end products could lead to more informative models. Another potential source of information on the metabolite level could be the concentrations of intermediates made during synthesis. Correlations between biosynthetic genes, and perhaps the metabolites as well, tend to decrease as distance in a pathway increases. Therefore, experiments that quantitate metabolic intermediates could be useful for finding biosynthetic genes, particularly genes directly acting on the intermediate.

The discovery of the NpN4OMT1 enzyme and its variants using the methods disclosed herein enables the future elucidation of other enzymes in the galanthamine biosynthetic pathway and other un-elucidated pathways using similar techniques. Genes that co-express with NpN4OMT can be identified and used as candidate genes for other steps in the galanthamine biosynthetic pathway. This will potentially be useful for earlier steps in the pathway, considering the tendency of expression correlations to decrease as distance in metabolic pathways increase. This enzyme discovery technique also validates the use of this workflow on uncharacterized metabolic pathways and provides an additional method for pathway discovery.

Besides engineering the galanthamine pathway in higher plants and algae in order to obtain galanthamine economically and in high yield, the present disclosure also encompasses galanthamine production in plant cell cultures, cell-free extracts, production in organisms such as transgenic fungi, yeasts, bacteria such as E. coli and B. subtilis, and the use of immobilized enzymes, etc.

Those skilled in the art will recognize, or be able to ascertain using no more than routine experimentation, many equivalents to the specific embodiments of the disclosure specifically described herein. Such equivalents are intended to be encompassed within the scope of the following claims.

What is claimed is:

1. A transgenic plant, comprising within its genome, and expressing, a heterologous nucleotide sequence, wherein the heterologous nucleotide sequence encodes for an enzyme, wherein the enzyme is selected from the group consisting of a class I O-methyltransferase, a P450, and a norbelladine synthase/reductase.

2. The transgenic plant of claim 1, wherein said class I O-methyltransferase is a 4′-O-methyltransferase.

3. The transgenic plant of claim 2, wherein said 4′-O-methyltransferase is a norbelladine 4′-O-methyltransferase.

4. The transgenic plant of claim 3, wherein said norbelladine 4′-O-methyltransferase converts norbelladine to 4′-O-methylnorbelladine.

5. The transgenic plant of claim 4, wherein said norbelladine 4′-O-methyltransferase is selected from the group consisting of NpN4OMT1 (SEQ ID NO:15), NpN4OMT2 (SEQ ID NO: 17), NpN4OMT3 (SEQ ID NO: 19), NpN4OMT4 (SEQ ID NO:21), and NpN4OMT5 (SEQ ID NO:23).

6. The transgenic plant of claim 1, wherein the P450 is selected from the group consisting of CYP96T1 (SEQ ID NO:26), CYP96T2 (SEQ ID NO:27), and CYP96T3 (SEQ ID NO:28).

7. The transgenic plant of claim 1, wherein the norbelladine synthase/reductase is SEQ ID NO:29.

8. The transgenic plant of claim 1, the genome of which further comprises a heterologous nucleotide sequence encoding a protein selected from the group consisting of a 4′-O-methyltransferase, a P450, a norbelladine synthase/reductase, an enzyme that condenses 3,4-dihydroxybenzaldehyde and tyramine to form norbelladine, an enzyme that converts 4′-O-methylnorbelladine to N-demethylnarwedine, an enzyme that converts N-demethylnarwedine to N-demethylgalanthamine, an enzyme that converts N-demethylgalanthamine to galanthamine, an enzyme that converts 4′-O-methylnorbelladine to Noroxomaritidine, an enzyme that converts Noroxomaritidine to hemanthamine, and an enzymes that convert(s) 4′-O-methylnorbelladine to lycorine.

9. The transgenic plant of claim 8, selected from the group consisting of a species of Galanthus, species of Brachypodium, species of Setaria, species of Populus, tobacco, corn, rice, soybean, cassava, canola (rapeseed), wheat, peanut, palm, coconut, safflower, sesame, cottonseed, sunflower, flax, olive, safflower, sugarcane, castor bean, switchgrass, Miscanthus, Camelina and Jatropha.

10. The transgenic plant of claim 9, wherein the species is Camelina.

11. The transgenic plant of claim 10, wherein the transgenic plant produces a biochemical compound, wherein the biochemical compound is selected from the group consisting of galanthamine, hemanthamine, and lycorine.

12. A method of making a transgenic plant, comprising the steps of:
a) inserting into the genome of a plant cell a heterologous nucleotide sequence comprising, operably linked for expression: (i) a promoter sequence; (ii) a nucleotide sequence encoding a protein selected from the group consisting of a 4′-O-methyltransferase, a P450, a norbelladine synthase/reductase, an enzyme that condenses 3,4-dihydroxybenzaldehyde and tyramine to form norbelladine, an enzyme that converts 4′-O-methylnorbelladine to N-demethylnarwedine, an enzyme that converts N-demethylnarwedine to N-demethylgalanthamine, an enzyme that converts N-demethylgalanthamine to galanthamine, an enzyme that converts 4′-O-methylnorbelladine to Noroxomaritidine, an enzyme that converts Noroxomaritidine to hemanthamine, and an enzymes that convert(s) 4′-O-methylnorbelladine to lycorine;
b) obtaining a transformed plant cell; and
c) regenerating from said transformed plant cell a genetically transformed plant, cells of which express said protein.

13. The method of claim 12, wherein the nucleotide sequence encoding a protein is selected from the group consisting of NpN4OMT1 (SEQ ID NO:15), NpN4OMT2 (SEQ ID NO: 17), NpN4OMT3 (SEQ ID NO: 19), NpN4OMT4 (SEQ ID NO:21), NpN4OMT5 (SEQ ID NO:23), CYP96T1 (SEQ ID NO:26), CYP96T2 (SEQ ID NO:27), and CYP96T3 (SEQ ID NO:28).

14. The method of claim 13, further comprising recovering a biochemical compound from said transgenic plant, wherein the biochemical compound is selected from the group consisting of galanthamine, hemanthamine, and lycorine.

15. A method of identifying genes in a biosynthetic pathway of an end product in an organism, comprising the steps of:
a) confirming the presence of said end product in a tissue or tissues of said organism;
b) identifying a gene or genes that co-expresses with accumulation of said end product;
c) identifying and characterizing previously characterized homologs or orthologues, or naturally occurring variants of said gene or genes of step b;
d) optionally, characterizing sequence motifs for one or more enzymes of step b or c;
e) expressing nucleotide sequences encoding one or more enzymes of step b or c, and isolating and characterizing said enzyme or enzymes;
f) optionally, performing phylogenetic analysis of said gene or genes identified in step c;
g) optionally, determining the expression profile of said gene or genes identified in step c.
